(19) United States
(12) Patent Application Publication
(10) Pub. No.: US 2026/0207783 A1
(43) Pub. Date: Jul. 23, 2026

(54) AAV FORMULATION

(71) Applicant: ENCODED THERAPEUTICS, INC., South San Francisco, CA (US)

(72) Inventors: Nathan Wise, South San Francisco, CA (US); Aashish Lamichhane, South San Francisco, CA (US); Travis Tran, South San Francisco, CA (US); Martin Moorhead, South San Francisco, CA (US)

(21) Appl. No.: 19/139,749

(22) PCT Filed: Dec. 28, 2023
(86) PCT No.: PCT/US2023/086286

§ 371 Date: Jun. 16, 2025

Related U.S. Application Data
(60) Provisional application No. 63/436,016, filed on Dec. 29, 2022.

(51) Int. Cl. A61K 48/00; A61K 38/17; A61K 47/02; A61K 47/10; A61K 47/26; C12N 15/86

(52) U.S. Cl. CPC A61K 48/0058; A61K 38/177; A61K 47/02; A61K 47/10; A61K 47/26; A61K 48/0075; A61K 48/0083; C12N 15/86; C12N 2750/14143

(57) ABSTRACT

The disclosure provides a composition comprising recombinant adeno-associated virus (AAV) vectors comprising a heterologous nucleic acid, sodium chloride, potassium chloride, magnesium chloride, phosphate buffer, and a non-ionic surfactant, at pH of 7.2-7.4. The disclosure further provides a method of treating a neurological disorder in a subject, the method comprising directly administering to the central nervous system of a subject in need thereof the composition.

### TECHNICAL FIELD

The disclosure relates to a composition comprising recombinant adeno-associated vectors.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: file name; “55330A_SeqListing.XML,” 175,534 bytes, created on Dec. 27, 2023.

### BACKGROUND

Adeno-associated virus (AAV) is a small, replication-defective, non-enveloped animal virus belonging to the family Parvoviridae. The AAV genome consists of a linear single stranded DNA which is ~4.7 kb in length. The genome consists of two open reading frames (ORF) flanked by an inverted terminal repeat (ITR) sequence that is about 145 bp in length. The ITR consists of a nucleotide sequence at the 5′ end (5′ ITR) and a nucleotide sequence located at the 3′ end (3′ ITR) that contain palindromic sequences. The ITRs function in cis by folding over to form T-shaped hairpin structures by complementary base pairing that function as primers during initiation of DNA replication for second strand synthesis. The two open reading frames encode for rep and cap genes that are involved in replication and packaging of the virion.

AAVs infect both dividing and quiescent cells. Due to the specificity, efficiency, and safety associated with AAVs, AAV vectors have emerged as an expression vector of choice for gene therapy applications. Production conditions and formulation of AAV vectors must be carefully selected to ensure vector stability, purity, and potency while maintaining patient safety. Delivery to the central nervous system presents unique challenges. The brain and spinal cord are compartmentalized organs, and compositions must have appropriate physicochemical properties tailored for administration to these regions in addition to avoiding inflammation and adverse immunogenic reactions. There is a need in the art for optimized, stable compositions comprising AAV vectors that are suitable for administration to the central nervous system.

### SUMMARY

The present disclosure provides a composition comprising recombinant adeno-associated virus (AAV) vectors comprising a heterologous nucleic acid, sodium chloride, potassium chloride, magnesium chloride, phosphate buffer, and a non-ionic surfactant, at pH of 7.2-7.4.

The present disclosure also provides a composition comprising recombinant adeno-associated virus (AAV) vectors comprising a heterologous nucleic acid, sodium chloride, potassium chloride, phosphate buffer, and a non-ionic surfactant, at pH of 7.2-7.4. The present disclosure further provides a composition comprising recombinant adeno-associated virus (AAV) vectors comprising a heterologous nucleic acid, sodium chloride, potassium chloride, magnesium chloride, Tris buffer, and a non-ionic surfactant, at pH of 7.2-7.4. The present disclosure further provides a composition comprising recombinant adeno-associated virus (AAV) vectors comprising a heterologous nucleic acid, sodium chloride, potassium chloride, Tris buffer, and a non-ionic surfactant, at pH of 7.2-7.4.

Optionally, the non-ionic surfactant is present at a concentration in the range of about 0.001% to about 0.01% (w/V). In various aspects, the non-ionic surfactant is a poloxamer, such as poloxamer 188. In this regard, the composition in various aspects comprises poloxamer 188 present at a concentration of about 0.005% (w/V). In various aspects, the composition comprises about 145 mM to about 150 mM sodium chloride and/or about 1.5 mM to about 4.5 mM potassium chloride and/or about 0.05 mM to about 1 mM magnesium chloride. For example, in some aspects, the composition comprises about 148 mM of sodium chloride, about 3 mM potassium chloride, and about 0.8 mM magnesium chloride. In various aspects, the phosphate buffer is present in an amount sufficient to provide about 0.5 mM to about 2 mM (e.g., about 1 mM) phosphate. Optionally, the phosphate buffer is sodium phosphate. In various aspects, the composition comprises about 5×10¹³ vg/mL to about 1×10¹⁴ vg/mL (e.g., about 8×10¹³ vg/mL) AAV. In some aspects, the composition does not comprise calcium, such as calcium chloride. In some aspects, the composition demonstrates a conductivity of about 15.0 to about 17.0 mS/cm.

The disclosure further provides a method of treating a neurological disorder in a subject, the method comprising directly administering to the central nervous system of a subject in need thereof the composition described herein. Optionally, the method comprises administering the composition to the subject via intracerebroventricular injection. Examples of neurological conditions suitable for treatment include, but are not limited to, Dravet syndrome and epilepsy. Use of the composition described herein to treat a subject in need thereof, and use of the composition described herein in the preparation of a medicament to treat a subject in need thereof, are contemplated. A kit comprising the composition described herein and instructions for use is provided.

Preferred embodiments of this disclosure are described herein, including the best mode known to the inventors for carrying out the disclosure. Variations of those preferred embodiments may become apparent to those of ordinary skill in the art upon reading the foregoing description. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Indeed, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document.

### BRIEF DESCRIPTION OF THE DRAWINGS

FIG. 1 illustrates percent loss of rAAV genomic DNA (vg), represented by a loss of absorbance at 260 nm (y-axis), in samples from Table 1 at time 0, after six freeze thaw cycles (“6 FT”), 7 or 17 days at room temperature (“RT d7” or “RT d17”), and 3 or 7 days at 37° C. (“37C d3” or “37C d7”).

FIG. 2 illustrates percent loss of rAAV capsid (cp), represented by a loss of absorbance at 280 nm (y-axis), in samples from Table 1 at time 0, after six freeze thaw cycles (“6 FT”), 7 or 17 days at room temperature (“RT d7” or “RT d17”), and 3 or 7 days at 37° C. (“RT d7” or “RT d17”).

FIG. 3 illustrates RP-HPLC results of a representative rAAV sample at t=0, incubation at room temperature (RT) for 17 days, and incubation at 37° C. for 7 days.

FIG. 4 illustrates fold-change in P1 (as per FIG. 3), for samples from Table 1 at time 0, after six freeze thaw cycles (“6×FT”), 7 or 17 days at room temperature (“RT d7” or “RT d17”), and 3 or 7 days at 37° C. (“37C d3” or “37C d7”).

FIG. 5 illustrates fold-change in P3 (as per FIG. 3), for samples from Table 1 at time 0, after six freeze thaw cycles (“6×FT”), 7 or 17 days at room temperature (“RT d7” or “RT d17”), and 3 or 7 days at 37° C. (“37C d3” or “37C d7”).

FIG. 6 illustrates fold-change in P5 (as per FIG. 3), for samples from Table 1 at time 0, after six freeze thaw cycles (“6×FT”), 7 or 17 days at room temperature (“RT d7” or “RT d17”), and 3 or 7 days at 37° C. (“37C d3” or “37C d7”).

FIG. 7 illustrates particle size (Z-average) for samples from Table 1. The bars in the graph correspond to, from left to right, Sample 1, Sample 2, Sample 3, Sample 4, Sample 5, Sample 6, Sample 7, Sample 8, Sample 9, Sample 10, and Sample 11.

FIG. 8 illustrates polydispersity index (PDI) for each sample from Table 1. The bars in the graph correspond to, from left to right, Sample 1, Sample 2, Sample 3, Sample 4, Sample 5, Sample 6, Sample 7, Sample 8, Sample 9, Sample 10, and Sample 11.

FIG. 9 illustrates vector genome titer as measured by ddPCR of samples from Table 2 at time 0, after 1 month at <−70° C., after 1 month or one week at room temperature, after 2 weeks or 1 month at 37° C., or after 10 freeze thaw cycles.

FIG. 10 illustrates aggregation as measured by SE-HPLC of samples from Table 2 at time 0, after 1 month at <−70° C., after 1 month or one week at room temperature, after 2 weeks or 1 month at 37° C., or after 10 freeze thaw cycles. Two bars are provided for each sample. The bar on the left for each sample corresponds to A260 high molecular weight species (HMWS) (%) and the bar on the right for each sample corresponds to A280 high molecular weight species (HMWS) (%).

FIG. 11 illustrates fold-change in P1 species by RP-HPLC of samples from Table 2 at time 0, after 1 month at <−70° C., after 1 month at room temperature, after 2 weeks or 1 month at 37° C., or after 10 freeze thaw cycles. Two bars are provided for each condition. The bar on the left for each condition corresponds to formula 1 and the bar on the right for each condition corresponds to formula 2.

FIG. 12 illustrates fold-change in P3 species by RP-HPLC of samples from Table 2 at time 0, after 1 month at <−70° C., after 1 month at room temperature, after 2 weeks or 1 month at 37° C., or after 10 freeze thaw cycles. Two bars are provided for each condition. The bar on the left for each condition corresponds to formula 1 and the bar on the right for each condition corresponds to formula 2.

FIG. 13 illustrates fold-change in P5 species by RP-HPLC of samples from Table 2 at time 0, after 1 month at <−70° C., after 1 month at room temperature, after 2 weeks or 1 month at 37° C., or after 10 freeze thaw cycles. Two bars are provided for each condition. The bar on the left for each condition corresponds to formula 1 and the bar on the right for each condition corresponds to formula 2.

FIG. 14 illustrates results of an in vitro potency assay for samples from Table 2 after 1 month at room temperature, after 2 weeks or 1 month at 37° C., or after 10 freeze thaw cycles, expressed as a percentage of potency at time 0.

FIG. 15 illustrates particle size for samples from Table 2 at time 0, after 1 week or 1 month at 4° C., after 1 month at room temperature, after 2 weeks or 1 month at 37° C., after 1 month at <−70° C., or after 10 freeze thaw cycles. Two bars are provided for each sample. The bar on the left for each sample corresponds to 15C Z-Ave diameter (nm) and the bar on the right for each sample corresponds to 95C Z-Ave diameter (nm).

FIG. 16 illustrates normalized polydispersity index for samples from Table 2 at time 0, after 1 week or 1 month at 4° C., after 1 month at room temperature, after 2 weeks or 1 month at 37° C., after 1 month at <−70° C., or after 10 freeze thaw cycles. Two bars are provided for each condition. The bar on the left for each condition corresponds to formula 1 and the bar on the right for each condition corresponds to formula 2.

FIG. 17 illustrates extrinsic fluorescence at 15° C. for samples from Table 2 at time 0, after 1 week or 1 month at 4° C., after 1 month at room temperature, after 2 weeks or 1 month at 37° C., after 1 month at <−70° C., or after 10 freeze thaw cycles. Two bars are provided for each condition. The bar on the left for each condition corresponds to formula 1 and the bar on the right for each condition corresponds to formula 2.

FIG. 18 illustrates Tₒₙₛₑₜ/Tₘ₁ (° C.) for samples from Table 2 at time 0, after 1 week or 1 month at 4° C., after 1 month at room temperature, after 2 weeks or 1 month at 37° C., after 1 month at <−70° C., or after 10 freeze thaw cycles. Two bars are provided for each condition. The bar on the left for each condition corresponds to formula 1 and the bar on the right for each condition corresponds to formula 2.

FIG. 19 illustrates Tₘ₂ (° C.) for samples from Table 2 at time 0, after 1 week or 1 month at 4° C., after 1 month at room temperature, after 2 weeks or 1 month at 37° C., after 1 month at <−70° C., or after 10 freeze thaw cycles. Two bars are provided for each condition. The bar on the left for each condition corresponds to formula 1 and the bar on the right for each condition corresponds to formula 2.

FIG. 20 illustrates subvisible particle analysis for Formulation buffer 1 alone (1B), Formulation buffer 1 with AAV (1S), Formulation buffer 2 alone (2B) and Formulation buffer 2 with AAV (2S) at time 0, after 1 month at <−70° C., after 1 week or 1 month at 4° C., after 1 month at room temperature, or after 2 weeks or 1 month at 37° C. Three bars are provided for each sample. The bar on the left for each sample corresponds to 2 microns, the middle bar corresponds to 10 microns, and the bar on the right for each sample corresponds to 25 microns.

FIG. 21 illustrates subvisible particle analysis for Formulation buffer 1 alone (1B), Formulation buffer 1 with AAV (1S), Formulation buffer 2 alone (2B), and Formulation buffer 2 with AAV (2S) after 10 freeze thaw cycles. Three bars are provided for each sample. The bar on the left for each sample corresponds to 2 microns, the middle bar corresponds to 10 microns, and the bar on the right for each sample corresponds to 25 microns.

FIG. 22 illustrates vector genome concentration (vg/mL) by ddPCR for formulations of Table 4 at time 0, after 10 freeze thaw cycles, or after seven or fourteen days at 37° C.

FIG. 23 illustrates statistical variation of Tₒₙₛₑₜ (° C.) at t=0 and stressed conditions for formulations of Table 4. The bars in the graph correspond to, from left to right, Sample 1, Sample 2, Sample 3, Sample 4, Sample 5, Sample 6, Sample 7, Sample 8, and Sample 9.

FIG. 24 illustrates extrinsic fluorescence (RFU (490-650 nm)) at 15° C. for formulations of Table 4 at time 0 (first bar for each sample), after 10 freeze thaw cycles (second bar for each sample), or after seven or fourteen days at 37° C. (third and fourth bars, respectively, for each sample).

### DETAILED DESCRIPTION

The present disclosure provides a stable composition which may be suitable for administering a gene therapy. Gene therapies may be delivered via a range of different vectors, for example viral vectors (e.g., lentiviral vectors, adeno viral vectors, and adeno-associated viral vectors) or non-viral vectors (e.g., naked DNA, particle based, and chemical based). In one example, the composition is suitable for administering AAV vectors. The compositions described herein may also be suitable for administration to the central nervous system of a subject in need thereof.

The composition may comprise recombinant adeno-associated virus (AAV) vectors comprising a heterologous nucleic acid and one or more of sodium chloride, potassium chloride, magnesium chloride, phosphate buffer, and a non-ionic surfactant, at pH of 7.2-7.4. In certain aspects, the composition comprises recombinant AAV vectors comprising a heterologous nucleic acid, sodium chloride, potassium chloride, magnesium chloride, phosphate buffer, and a non-ionic surfactant, at pH of 7.2-7.4. In various aspects, the composition comprises recombinant AAV vectors comprising a heterologous nucleic acid, sodium chloride, potassium chloride, magnesium chloride, phosphate buffer, and a non-ionic surfactant, at pH of 7.30. In various aspects, the composition comprises recombinant AAV vectors comprising a heterologous nucleic acid, sodium chloride, potassium chloride, magnesium chloride, phosphate buffer, trehalose, and a non-ionic surfactant, at pH of 7.2-7.4. In various aspects, the composition comprises recombinant AAV vectors comprising a heterologous nucleic acid, sodium chloride, potassium chloride, magnesium chloride, phosphate buffer, trehalose, and a non-ionic surfactant, at pH of 7.30. Features of the composition are further described below.

Surfactants improve stability of compositions by, e.g., minimizing surface-induced degradation. Hydrophobic portions of surfactant molecules occupy interfacial positions (e.g., air/liquid), while hydrophilic portions of the molecules remain oriented toward the bulk solvent. Pharmaceutically acceptable non-ionic surfactants include, but are not limited to, Polysorbate 80 (Tween 80; PS80), Polysorbate 20 (Tween 20; PS20), digitonin, Triton X-100, Triton X-144, and poloxamers. Poloxamers, also known as Pluronics®, are amphiphilic block copolymers of poly(ethylene oxide) (PEO) and poly(propylene oxide) (PPO). Bodratti et a., J Funct Biomater. 2018 March; 9(1): 11. The original manufacturer of Pluronics®, BASF, introduced a specific nomenclature wherein the first letter indicates the physical state (Paste (P), Liquid (L), or Flake (F)), and a series of numbers, wherein the first one or two numbers relate to the molecular weight and the last number indicates the weight percent of the PEO block. Commercially available Pluronics® include, e.g., L64, P65, P84, P85, F88, P103, P104, P105, F108, P123, F127. In various aspects, the non-ionic surfactant in the composition is a poloxamer. In various aspects, the non-ionic surfactant is poloxamer 188.

Optionally, the composition of the present disclosure comprises about 0.001% (w/v) to about 0.02%, such as about 0.001% (w/v) to about 0.01% (w/v), or about 0.001% to about 0.005%, or about 0.0025% (w/v) to about 0.0075% (w/v), or about 0.003% (w/v) to about 0.007% (w/v), or about 0.004% (w/v) to about 0.006% (w/v), or about 0.003% to about 0.005%, or about 0.003% to about 0.0046% non-ionic surfactant. In exemplary aspects, the composition comprises about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), about 0.0095% (w/v), about 0.01% (w/v), about 0.015% (w/v), or about 0.02% (w/v) non-ionic surfactant (as well as ranges comprising any of these values as endpoints). In certain embodiments, the composition of the present disclosure comprises about 0.005% (w/v) non-ionic surfactant, such as poloxamer (e.g., poloxamer 188).

The composition of the disclosure further comprises one or more pharmaceutically acceptable salts. Suitable “pharmaceutically acceptable salts” include, but are not limited to, metal salts (e.g., sodium, potassium, and cesium salts) and alkaline earth metal salts (e.g., magnesium salts). Non-limiting examples of pharmaceutically acceptable salts include, without limitation, sodium salts, magnesium salts, and potassium salts (e.g., sodium chloride, magnesium chloride, and potassium chloride; sodium acetate, magnesium acetate, and potassium acetate; sodium citrate, magnesium citrate, and potassium citrate; sodium phosphate, magnesium phosphate, and potassium phosphate; sodium fluoride, magnesium fluoride, and potassium fluoride; sodium bromide, magnesium bromide, and potassium bromide; and sodium iodide, magnesium iodide, and potassium iodide). In various aspects, the formulation comprises one or more of sodium chloride, magnesium chloride, and potassium chloride; optionally, the formulation comprises sodium chloride, magnesium chloride, and potassium chloride. In some aspects, the formulation is substantially free of magnesium chloride.

In exemplary aspects, the composition of the present disclosure comprises about 0 mM to about 200 mM, about 1 mM to about 175 mM, about 3 mM to about 150 mM, or about 5 mM to about 100 mM of a pharmaceutically acceptable salt. In various aspects, the composition comprises about 0.1 mM to about 10 mM, about 0.5 mM to about 8 mM, about 0.5 mM to about 6.5 mM about 0.5 mM to about 3 mM, about 0.5 mM to about 1 mM, about 0.7 mM to about 0.9 mM of a pharmaceutically acceptable salt, such as any one of the pharmaceutically acceptable salt described above (e.g., magnesium chloride). In various aspects, the composition comprises about 1 mM to about 10 mM, about 1 mM to about 5 mM, 0.5 mM to about 6.5 mM, about 1.5 mM to about 4.5 mM, or about 2 mM to about 4 mM of a pharmaceutically acceptable salt, such as any one of the pharmaceutically acceptable salt described above (e.g., potassium chloride). In various aspects, the composition comprises about 100 mM to about 175 mM, about 120 mM to about 160 mM, about 130 mM to about 150 mM, about 140 mM to about 150 mM, or about 145 mM to about 150 mM of a pharmaceutically acceptable salt, such as any one of the pharmaceutically acceptable salt described above (e.g., sodium chloride).

In exemplary aspects, the composition comprises about 0.1 mM, about 0.5 mM, about 0.8 mM, about 1 mM, about 2 mM, about 3 mM, about 4 mM, about 5 mM, or about 10 mM of one or more pharmaceutically acceptable salts. In exemplary aspects, the composition comprises about 120 mM, about 125 mM, about 130 mM, about 135 mM, about 140 mM, about 145 mM, about 150 mM, about 155 mM, about 165 mM, or about 170 mM of one or more pharmaceutically acceptable salts. In certain embodiments, composition comprises about 145 mM to about 150 mM (e.g., about 148 mM) of sodium chloride, about 1.5 mM to about 4.5 mM (e.g., about 3 mM) potassium chloride, and about 0.05 mM to about 1 mM (e.g., about 0.8 mM) magnesium chloride.

The composition further comprises a buffering agent. Pharmaceutically acceptable buffering agents are well known in the art, and include without limitation, phosphate buffers (e.g., sodium phosphate), histidine, citrate buffers (e.g., sodium citrate), HEPES, Tris, glycine, acetate buffers (e.g., sodium acetate), sodium carbonate, lysine, arginine, and mixtures thereof. In some embodiments the composition may comprise two or more buffering agents. In exemplary embodiments, the buffer is a phosphate buffer, such as a sodium phosphate buffer, optionally provided as sodium phosphate (e.g., sodium phosphate monobasic and/or sodium phosphate dibasic). In some cases a phosphate buffer may comprise potassium phosphate. In this regard, the composition optionally comprises phosphate buffer in an amount sufficient to provide about 0.01 mM to about 10 mM, about 0.05 mM to about 5 mM, about 0.1 mM to about 3 mM, about 3 mM to about 7 mM, or about 0.5 mM to about 2 mM phosphate. In some aspects, the composition optionally comprises phosphate buffer in an amount sufficient to provide about 0.5 mM, about 1 mM, about 1.5 mM, about 2 mM, about 2.5 mM, about 3, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, about 10 mM, about 10.5 mM, about 11 mM, about 11.5 mM, about 12 mM, about 12.5 mM, about 13, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, or about 20 mM phosphate. In various aspects, the composition comprises about 1 mM phosphate, optionally provided as sodium phosphate (e.g., sodium phosphate monobasic and/or sodium phosphate dibasic). In various aspects, the composition comprises about 5 mM phosphate, optionally provided as sodium phosphate (e.g., sodium phosphate monobasic and/or sodium phosphate dibasic).

In exemplary embodiments, the buffer is a Tris buffer, optionally provided as a Tris hydrochloride or a Tris acetate salt. In this regard, the composition optionally comprises Tris buffer in an amount sufficient to provide about 0.01 mM to about 30 mM, about 0.01 mM to about 20 mM, about 0.01 mM to about 10 mM, about 0.05 mM to about 5 mM, about 0.1 mM to about 3 mM, about 3 mM to about 7 mM, or about 0.5 mM to about 2 mM Tris. In some aspects, the composition optionally comprises Tris buffer in an amount sufficient to provide about 0.5 mM, about 1 mM, about 1.5 mM, about 2 mM, about 2.5 mM, about 3, about 3.5 mM, about 4 mM, about 4.5 mM, about 5 mM, about 5.5 mM, about 6 mM, about 6.5 mM, about 7 mM, about 7.5 mM, about 8 mM, about 8.5 mM, about 9 mM, about 9.5 mM, about 10 mM, about 10.5 mM, about 11 mM, about 11.5 mM, about 12 mM, about 12.5 mM, about 13, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, or about 20 mM Tris. In various aspects, the composition comprises about 1 mM Tris. In various aspects, the composition comprises about 5 mM Tris.

The composition has a physiologically compatible pH. For example, the pH of the composition is about 6.5 to about 9.0, about 6.5 to about 8.0, about 6.9 to about 7.7, about 6.9 to about 7.4, about 7.0 to about 7.5, about 7.0 to about 7.4, about 7.2 to about 7.4, about 7.0 to about 7.3, about 7.1 to about 7.4, or about 7.2 to about 7.5. In various embodiments, the pH of the formulation is about 7.0, about 7.1, about 7.2, about 7.3, about 7.4, about 7.5, about 7.6, about 7.7, or about 7.8. In exemplary aspects, the pH of the composition is about 7.2, about 7.3, or about 7.4. In certain embodiments, the pH of the composition is about 7.3.

In various aspects, the composition does not comprise a saccharide (e.g., monosaccharide, disaccharide, cyclic polysaccharide, sugar alcohol, linear branched dextran, or linear non-branched dextran, such as sucrose, trehalose, glucose, mannitol, or sorbitol). In various aspects, the composition does not comprise amino acids, such as glycine, glutamine, asparagine, arginine, or lysine. In various aspects, the formulation does not comprise calcium, such as calcium chloride.

The composition optionally comprises recombinant adeno-associated virus (AAV) vectors, such as recombinant AAV vectors comprising a heterologous nucleic acid. The abbreviation “rAAV” refers to recombinant adeno-associated virus. The term “AAV” includes all serotypes of AAV, including AAV1, AAV2, AAV3, AAV4, AAV5, AAV6, AAV7, AAV8, AAV9, AAV9.47, AAV9(hul4), AAV10, AAV11, AAV 12, AAV13, AAVrh8, AAVrhlO, AAV-DJ, and AAV-DJ8, and hybrids thereof (i.e., chimeric AAV vectors). The genomic sequences of various serotypes of AAV, as well as the sequences of the native terminal repeats (TRs), Rep proteins, and capsid subunits are known in the art. Such sequences may be found in the literature or in public databases such as GenBank. By “heterologous nucleic acid” is meant a polynucleotide sequence not of AAV origin, typically a sequence of interest for delivery to a host cell. In general, the heterologous polynucleotide is flanked by at least one, and generally by two, AAV inverted terminal repeat sequences (ITRs). The term AAV vector as used herein encompasses both AAV particles (i.e., a viral particle composed of at least one AAV capsid protein and an encapsidated polynucleotide) and AAV vector plasmids (i.e., a polynucleotide comprising AAV components that are not encapsulated into AAV coat proteins). An AAV vector may either be single-stranded (ssAAV) or self-complementary (scAAV). See, e.g., Raj et al., Expert Rev Hematol. 2011 October; 4(5): 539-549. AAVs may comprise genome components and capsids from multiple serotypes (e.g., pseudotyped vectors). For example, an AAV may comprise the genome of serotype 2 (e.g., ITRs) packaged in the capsid from serotype 5 or serotype 9. Pseudotyped vectors may demonstrate improved transduction efficiency as well as altered tropism. In some cases, an AAV serotype that can cross the blood brain barrier or infect cells of the CNS is preferred. In some aspects, the recombinant AAV vector is AAV1, AAV8, AAV9, AAVDJ, or chimeric AAV comprising features of two or more of these serotypes. In various embodiments, the AAV vector is an AAV9 vector or an scAAV9 vector. In certain embodiments, the AAV vector is an AAV9 vector or an scAAV9 vector and comprises a heterologous nucleic acid flanked by ITRs from a AAV serotype other than AAV9. In certain embodiments, the AAV vector is an AAV9 vector or an scAAV9 vector and comprises a heterologous nucleic acid flanked by AAV serotype 2 ITRs (i.e., ITR2).

In exemplary aspects, the composition comprises at least about 1×10⁷ AAV viral genomes (vg), at least about 1×10⁸ vg, at least about 1×10⁹ vg, at least about 1×10¹⁰ vg, at least about 1×10¹¹ vg, at least about 1×10¹² vg, at least about 5×10¹² vg, at least about 6×10¹² vg, at least about 7×10¹² vg, at least about 8×10¹² vg, at least about 9×10¹² vg, at least about 9.5×10¹² vg, at least about 9.8×10¹² vg, at least about 1×10¹³ vg, at least about 1.5×10¹³ vg, at least about 1.6×10¹³ vg, at least about 1.7×10¹³ vg, at least about 1.8×10¹³ vg, at least about 1.9×10¹³ vg, at least about 2×10¹³ vg, at least about 3×10¹³ vg, at least about 4×10¹³ vg, at least about 5×10¹³ vg, at least about 6×10¹³ vg, at least about 7×10¹³ vg, at least about 8×10¹³ vg, at least about 9×10¹³ vg, at least about 1×10¹⁴ vg, at least about 2×10¹⁴ vg, at least about 3×10¹⁴ vg, at least about 4×10¹⁴ vg, at least about 5×10¹⁴ vg, at least about 6×10¹⁴ vg, at least about 7×10¹⁴ vg, at least about 8×10¹⁴ vg, at least about 9×10¹⁴ vg, or at least about 1×10¹⁵ vg. In various aspects, the composition comprises about 1×10¹² vg/mL to about 5×10¹⁴ vg/mL, about 1×10¹³ vg/mL to about 5×10¹⁴ vg/mL, about 5×10¹² vg/mL to about 1×10¹⁴ vg/mL, about 5×10¹³ vg/mL to about 1×10¹⁴ vg/mL, about 7×10¹³ vg/mL to about 9×10¹³ vg/mL of AAV. In various aspects, the composition comprises about 1×10¹³ vg/mL, about 2×10¹³ vg/mL, about 3×10¹³ vg/mL, about 4×10¹³ vg/mL, about 5×10¹³ vg/mL, about 6×10¹³ vg/mL, about 7×10¹³ vg/mL, about 8×10¹³ vg/mL, about 9×10¹³ vg/mL, about 1×10¹⁴ vg/mL, about 2×10¹⁴ vg/mL, about 3×10¹⁴ vg/mL, about 4×10¹⁴ vg/mL, or about 5×10¹⁴ vg/mL of AAV. In various aspects, the composition comprises about 5×10¹³ vg/mL to about 1×10¹⁴ vg/mL (e.g., about 8×10¹³ vg/mL) AAV. In various aspects, the composition comprises about 7×10¹² vg/mL to about 1.3×10¹³ vg/mL, about 1.4×10¹³ vg/mL to about 2.6×10¹³ vg/mL, or about 9.8×10¹² vg/mL to about 1.82×10¹³ vg/mL.

The present disclosure provides a composition comprising (a) recombinant adeno-associated virus (AAV) vectors comprising a heterologous nucleic acid, optionally about 5×10¹³ vg/mL to about 1×10¹⁴ vg/mL (e.g., about 8×10¹³ vg/mL) of the recombinant AAV vectors; (b) sodium chloride, optionally about 145 mM to about 150 mM sodium chloride; (c) potassium chloride, optionally about 1.5 mM to about 10 mM potassium chloride (e.g., about 1.5 mM to about 4.5 mM); (d) magnesium chloride, optionally about 0.05 mM to about 1 mM magnesium chloride; (e) phosphate buffer (e.g., sodium phosphate), optionally present in an amount sufficient to provide about 0.5 mM to about 2 mM (e.g., about 1 mM) phosphate; and (f) a non-ionic surfactant (e.g., a poloxamer, such as poloxamer 188), optionally present at a concentration in the range of about 0.001% to about 0.01% (w/V); at pH of 7.2-7.4. For instance, the surfactant may be poloxamer 188 present at a concentration of about 0.005% (w/V). In some aspects, the composition does not comprise calcium, such as calcium chloride. In some aspects, the composition demonstrates a conductivity of about 15.0 to about 17.0 mS/cm.

The present disclosure provides a composition comprising (a) recombinant adeno-associated virus (AAV) vectors comprising a heterologous nucleic acid, optionally about 5×10¹³ vg/mL to about 1×10¹⁴ vg/mL (e.g., about 8×10¹³ vg/mL) of the recombinant AAV vectors; (b) sodium chloride, optionally about 145 mM to about 150 mM sodium chloride; (c) potassium chloride, optionally about 1.5 mM to about 10 mM potassium chloride (e.g., about 1.5 mM to about 4.5 mM); (d) phosphate buffer (e.g., sodium phosphate), optionally present in an amount sufficient to provide about 0.5 mM to about 2 mM (e.g., about 1 mM) phosphate; and (e) a non-ionic surfactant (e.g., a poloxamer, such as poloxamer 188), optionally present at a concentration in the range of about 0.001% to about 0.01% (w/V); at pH of 7.2-7.4. For instance, the surfactant may be poloxamer 188 present at a concentration of about 0.005% (w/V). In some aspects, the composition does not comprise calcium, such as calcium chloride. In some aspects, the composition demonstrates a conductivity of about 15.0 to about 17.0 mS/cm.

The present disclosure provides a composition comprising (a) recombinant adeno-associated virus (AAV) vectors comprising a heterologous nucleic acid, optionally about 5×10¹³ vg/mL to about 1×10¹⁴ vg/mL (e.g., about 8×10¹³ vg/mL) of the recombinant AAV vectors; (b) sodium chloride, optionally about 145 mM to about 150 mM sodium chloride; (c) potassium chloride, optionally about 1.5 mM to about 4.5 mM potassium chloride; (d) Tris buffer, optionally present in an amount sufficient to provide about 0.5 mM to about 10 mM (e.g., about 1 mM, or about 5 mM) phosphate; and (e) a non-ionic surfactant (e.g., a poloxamer, such as poloxamer 188), optionally present at a concentration in the range of about 0.001% to about 0.01% (w/V); at pH of 7.2-7.4. For instance, the surfactant may be poloxamer 188 present at a concentration of about 0.005% (w/V). In some aspects, the composition does not comprise calcium, such as calcium chloride. In some aspects, the composition demonstrates a conductivity of about 15.0 to about 17.0 mS/cm. In some aspects, the composition further comprises magnesium chloride, optionally about 0.05 mM to about 1 mM magnesium chloride.

The disclosure further contemplates a composition comprising the components described herein (e.g., sodium chloride, potassium chloride, optionally magnesium chloride, phosphate buffer, and a non-ionic surfactant) but not comprising rAAV. In this respect, the present disclosure provides a composition comprising (a) sodium chloride, optionally about 145 mM to about 150 mM sodium chloride; (b) potassium chloride, optionally about 1.5 mM to about 4.5 mM potassium chloride; (c) magnesium chloride, optionally about 0.05 mM to about 1 mM magnesium chloride; (d) phosphate buffer (e.g., sodium phosphate), optionally present in an amount sufficient to provide about 0.5 mM to about 10 mM (e.g., about 1 mM or about 5 mM) phosphate; and (e) a non-ionic surfactant (e.g., a poloxamer, such as poloxamer 188), optionally present at a concentration in the range of about 0.001% to about 0.01% (w/V); at pH of 7.2-7.4.

In other respects, the present disclosure provides a composition comprising (a) sodium chloride, optionally about 145 mM to about 150 mM sodium chloride; (b) potassium chloride, optionally about 1.5 mM to about 4.5 mM potassium chloride; (c) magnesium chloride, optionally about 0.05 mM to about 1 mM magnesium chloride; (d) Tris buffer, optionally present in an amount sufficient to provide about 0.5 mM to about 10 mM (e.g., about 1 mM or about 5 mM) Tris; and (e) a non-ionic surfactant (e.g., a poloxamer, such as poloxamer 188), optionally present at a concentration in the range of about 0.001% to about 0.01% (w/V); at pH of 7.2-7.4. In this respect, the present disclosure provides a composition comprising (a) sodium chloride, optionally about 145 mM to about 150 mM sodium chloride; (b) potassium chloride, optionally about 1.5 mM to about 4.5 mM potassium chloride; (c) phosphate buffer (e.g., sodium phosphate), optionally present in an amount sufficient to provide about 0.5 mM to about 10 mM (e.g., about 1 mM or about 5 mM) phosphate; and (d) a non-ionic surfactant (e.g., a poloxamer, such as poloxamer 188), optionally present at a concentration in the range of about 0.001% to about 0.01% (w/V); at pH of 7.2-7.4 . . . . In other respects, the present disclosure provides a composition comprising (a) sodium chloride, optionally about 145 mM to about 150 mM sodium chloride; (b) potassium chloride, optionally about 1.5 mM to about 4.5 mM potassium chloride; (c) Tris buffer, optionally present in an amount sufficient to provide about 0.5 mM to about 10 mM (e.g., about 1 mM or about 5 mM) Tris; and (d) a non-ionic surfactant (e.g., a poloxamer, such as poloxamer 188), optionally present at a concentration in the range of about 0.001% to about 0.01% (w/V); at pH of 7.2-7.4.

In various examples, the surfactant is poloxamer 188 present at a concentration of about 0.005% (w/V). In some aspects, the composition does not comprise calcium, such as calcium chloride.

In some aspects, the composition may be such that the rAAV particles present in the composition are substantially stable in the composition. For example, vector genome titer as measured by ddPCR may vary by less than 30%, less than 25%, less than 20%, less than 15%, less than 10%, less than 9%, less than less than 8%, less than 7%, less than 6%, less than 5%, less than 4%, less than 3%, less than 2%, or less than 1% after exposure to storage and/or handling conditions. Examples of storage or handling conditions include, but are not limited to, storage at about 4° C., storage at about room temperature, storage at about 37° C., or repeated freeze thaw cycles. In certain aspects, the composition is substantially stable after storage up to an extended period of time. For instance, storage can be for less than 24 hours, about 1-2 days, about 2-5 days, about 5-7 days, about 1-2 weeks, about 2-4 weeks, up to a month, or for more than a month. In specific aspects, the composition may be substantially stable after exposure to storage at 4° C. for up to a month, storage at room temperature for up to a month, storage at 37° C. for up to two weeks or up to a month. In other aspects, the composition is substantially stable after one or more freeze thaw cycles. For instance, the composition is substantially stable after two, three, four, five, six, seven, eight, nine, ten, twelve, fifteen, twenty, twenty-five, or more than twenty-five repeated freeze thaw cycles.

Stability may also be assessed by aggregation as determined by measured by size exclusion high performance liquid chromatography (SE-HPLC). For example, the rAAVs of a formulation as described herein may show a less than 30%, less than 25%, less than 20%, less than 15%, less than 10%, less than 9%, less than less than 8%, less than 7%, less than 6%, less than 5%, less than 4%, less than 3%, less than 2%, or less than 1% increase in aggregation after exposure to conditions such as those described above (e.g., storage at 4° C., storage at room temperature, storage at 37° C., or repeated freeze thaw cycles). Reversed-Phase HPLC (RP-HPLC) may also be used to compare prevalence of different species before and after stress. For example, the rAAVs of a formulation as described herein may show a less than 30%, less than 25%, less than 20%, less than 15%, less than 10%, less than 9%, less than less than 8%, less than 7%, less than 6%, less than 5%, less than 4%, less than 3%, less than 2%, or less than 1% increase in a RP-HPLC species after exposure to conditions such as those described above (e.g., storage at 4° C., storage at room temperature, or repeated freeze thaw cycles). In vitro potency may also be used to assess rAAV stability. For example, the rAAVs of a formulation as described herein may show a less than 30%, less than 25%, less than 20%, less than 15%, less than 10%, less than 9%, less than less than 8%, less than 7%, less than 6%, less than 5%, less than 4%, less than 3%, less than 2%, or less than 1% decrease in in vitro potency after exposure to conditions such those described above (e.g., storage at 4° C., storage at room temperature, or repeated freeze thaw cycles).

The recombinant AAV vector comprises a heterologous nucleic acid. The heterologous nucleic acid may comprise, or be in the form, of an “expression cassette,” referring to a polynucleotide comprising one or more regulatory elements operably linked to a coding sequence (i.e., a polynucleotide sequence encoding an RNA or peptide of interest). The recombinant AAV vector may comprise any heterologous nucleic acid of interest, including a transgene encoding a peptide or protein of interest. Transgenes as referenced herein generally do not contain introns, or do not contain more than one intron, although this is not required. A transgene can be obtained from a cDNA sequence rather than from genomic sequence. In some instances, the transgene encodes an ion channel, a neurotransmitter regulator, a transcription factor, or a subunit, variant, or functional fragment of any of the foregoing. Examples of ion channels include voltage gated and ligand gated ion channels. Voltage gated ion channels include sodium channels, calcium channels, potassium channels, and proton channels. In some embodiments, the transgene encodes SCN1A. In some instances, the transgene encodes a subunit of a voltage gated sodium channel, e.g., a sodium ion channel alpha subunit, sodium ion channel beta subunit, or a variant or functional fragment thereof. An example of a voltage gated sodium channel subunit is SCNIA (NM_001165963.1).

In some embodiments, the transgene encodes a polypeptide comprising an amino acid sequence having at least 80%, at least 85%, at least 90%, at least 95%, at least 96%, at least 97%, at least 98%, or at least 99% sequence identity to the amino sequence of SCN1A, or a functional fragment thereof. In this regard, the transgene optionally encodes a polypeptide comprising an amino acid sequence having at least 80%, at least 85%, at least 90%, at least 95%, at least 96%, at least 97%, at least 98%, or at least 99% sequence identity to SEQ ID NO: 6.

Another example of a heterologous nucleic acid of interest encodes a transcription factor, which may be a transcription activator or a transcription repressor. A transcription factor comprises a DNA binding domain and a transcription modulation domain. A DNA binding domain binds a transcription factor binding site in target DNA. A transcription modulation domain (TMD) contains binding sites for other proteins that promote or repress transcription of a target nucleic acid sequence. The TMD may contact transcriptional machinery (e.g., RNA polymerase) either directly or through other proteins (known as coactivators or comodulators). The transcription factor may be wildtype (i.e., unmodified) or may be a non-naturally occurring transcription factor, such as a transcription factor engineered such that, e.g., a DNA binding domain is operably linked to a transcription modulation domain to which the DNA binding domain is not naturally linked (e.g., derived from a different transcription factor or from a different species). In various aspects, the heterologous nucleic acid encodes a transcription factor that modulates expression (e.g., enhances expression) of SCN1A.

Examples of DNA binding domains include zinc fingers, helix-turn-helix, leucine zipper (e.g., bZIP), helix-loop-helix, and beta-scaffold Cas9, a Cas family protein, dCas9, a dCas family protein, or a transcriptional activator like effector (TALE). In some cases, the transgene is a DNA binding protein comprising a DNA cleaving region that has been deactivated. In some cases, the transgene comprises a gene editing protein, e.g., a Cas protein, Cas9. The heterologous nucleic acid may encode multiple copies of the same DNA binding domain, or may comprise multiple DNA binding domains of different sequences. For example, various aspects of the disclosure provide a heterologous nucleic acid comprising from 2 to 10 DNA binding domains, such as zinc fingers (e.g., 3 to 8 zinc fingers, or 1, 2, 3, 4, 5, 6, 7, 8, 9, or 10 zinc fingers).

Examples of suitable DNA binding domains are DNA binding domains having at least 80%, at least 85%, at least 90%, at least 95%, at least 96%, at least 97%, at least 98%, or at least 99% sequence identity to any of SEQ ID NOs: 7-28. In exemplary aspects, the DNA binding domain comprises the sequence any one of SEQ ID NOs: 7-28. See also International Patent Publication No. 2020/243651, incorporated herein by reference.

The TMD(s) and DNA binding domain(s) (DBD) may be derived from different proteins. An engineered TF may comprise more than one TMD, and two or more of the TMDs may be derived (e.g., isolated from) different proteins compared to other TMD(s) in the protein. In various aspects, the TMD is a transactivation domain, which enhances or upregulates expression. Examples of transactivation domains include, e.g., VP64 (SEQ ID NO: 29), VPR (SEQ ID NO: 30), VP16, VP128, p65, p300, CBP/p300-interacting transactivator 2 (CITED2) (SEQ ID NO: 31 or 32), CBP/p300-interacting transactivator 4 (CITED4) (SEQ ID NO: 33 or 34), EGR1 (SEQ ID NO: 35), or EGR3 (SEQ ID NO: 36). See also International Patent Publication No. WO 2019/109051, incorporated herein by reference in its entirety and in particular with respect to disclosure of transactivation domains and DNA binding domains. Any suitable arrangement of one or more DNA binding domains and one or more transcription modulation domains is contemplated. For example, the non-naturally occurring transcription factor optionally comprises a DNA binding domain (DBD) operably linked to at least two transcription modulation domains (TMD) in the following manner: TMD1-TMD2-DBD, DBD-TMD3-TMD4, or TMD1-TMD2-DBD-TMD3-TMD4. In certain embodiments, TMD1, TMD2, TMD3, and TMD4 are independently selected from the following: VP16, VP64, Viper, CITED2, CITED4, and CREB3, or functional fragments of any of the foregoing. Optionally, TMD1 and TMD2 are the same TMD. Optionally, TMD3 and TMD4 are the same TMD. In various aspects, TMD1, TMD2, TMD3 and TMD4 are the same TMD. In various aspects, a linker is present between two or more of the TMDs. Examples of suitable linkers include, but are not limited to, GS, GGSGGGSG (SEQ ID NO: 37) or GGS GGGS GGGS G (SEQ ID NO: 38). In a representative example, the heterologous nucleic acid encodes a zinc finger DNA-binding domain (optionally comprising multiple zinc fingers) or a transcription factor-like effector DNA-binding domain fused to transcription modulation domain (e.g., VP16 or VP64), wherein the DNA binding domain-transcription modulation domain fusion comprises one more linkers selected from GGSGGGSG (SEQ ID NO: 37) or GGS GGGS GGGS G (SEQ ID NO: 38).

Native transcription factors may be active in most cell types. Transcription factors also may be tissue-specific, such as those from muscle cells (e.g., MyoD and muscle enhancer factor 2 (MEF2)) or those from neuronal cells (e.g., nuclear factor 1C (NF1C), nuclear factor 1X (NF1X), Brain-1 (Brn-1), or Brain-2 (Brn-2)). Transcription factors also may be ligand-dependent. Ligand-dependent transcription factors comprise an additional domain which is bound by the ligand, which results in up- or down-regulation of gene expression. Steroid hormone receptors and nuclear receptors are examples of ligand-dependent transcription factors. Other examples of ligand-dependent transcription factors are metal-responsive transcription factors that, e.g., regulate metal (iron, zinc, or copper) homeostasis.

Examples of transcription factors include, but are not limited to, AF-4 transcription factors, Androgen receptor transcription factors, AP-2 transcription factors, ARID transcription factors, bHLH transcription factors, C/EBP transcription factors, CBF transcription factors, CG-1 transcription factors, COE transcription factors, COUP transcription factors, CP2 transcription factors, CSD transcription factors, CSL transcription factors, CTF/NFI transcription factors, CUT transcription factors, DM transcription factors, E2F transcription factors, EAF2 transcription factors, Ecdystd receptor transcription factors, ETS transcription factors, Fork head transcription factors, GCM transcription factors, GCR transcription factors, GTF21 transcription factors, HMG transcription factors, HMGI/HMGY transcription factors, Homeobox transcription factors, HSF transcription factors, HTH transcription factors, IRF transcription factors, MBD transcription factors, MH1 transcription factors, MYB transcription factors, NDT80/PhoG transcription factors, NF-YA transcription factors, NF-YB/C transcription factors, Nrf1 transcription factors, Nuclear orphan receptor transcription factors, Oestrogen receptor transcription factors, P53 transcription factors, PAX transcription factors, PC4 transcription factors, POU transcription factors, PPAR receptor transcription factors, PREB transcription factors, Progesterone receptor transcription factors, Prox1 transcription factors, Retinoic acid receptor transcription factors, RFX transcription factors, RHD transcription factors, ROR receptor transcription factors, Runt transcription factors, SAND transcription factors, SPZ1 transcription factors, SRF transcription factors, STAT transcription factors, T-box transcription factors, TEA transcription factors, TF-bZIP transcription factors, TF-Otx transcription factors, THAP transcription factors, Thyroid hormone receptor transcription factors, TSC22 transcription factors, Tub transcription factors, ZBTB transcription factors, zf-BED transcription factors, zf-C2H2 transcription factors, zf-C2HC transcription factors, zf-GATA transcription factors, zf-LITAF-like transcription factors, zf-MIZ transcription factors, and zf-NF-X1 transcription factors. Metal-responsive transcription factors include, but are not limited to, Aft1, Aft2, Fep1, SREA, Urbs1, Ace1, Amt1, Srf1, Mac1, Cuf1, GRISEA, Crr1, Zap1, and metal response element-binding transcription factor-1 (MTF-1). MTF-1 induces expression of metallothioneins and other genes involved in metal homeostasis in response to heavy metals such as copper. See, e.g., Rutherford and Bird, Eukaryot Cell. 2004 February; 3(1): 1-13; and Wang et al., Biol Chem. 2004 July; 385(7):623-32.

The transcription factor may be any of the transcription factors disclosed herein, or may comprise components of any of the referenced transcription factors referenced herein (e.g., the DNA binding domain or the transcription modulation domain of the referenced transcription factors). In exemplary aspects of the disclosure, the heterologous nucleic acid encodes a transcription factor that upregulates SCN1A production and is any of the engineered transcription factors described in International Patent Publication No. WO 2020/243651, incorporated herein by reference in its entirety. For example, in an exemplary embodiment, the engineered transcription factor comprises a DNA binding domain comprising a zinc finger motif having the following structure: LEPGEKP-[YKCPECGKSFS X HQRTH TGEKP]n-YKCPECGKSFS X HQRTH-TGKKTS (SEQ ID NO: 39), wherein n is an integer from 1-15, e.g., 1, 2, 3, 4, 5, 6, 7, 8, 9, 10, 11, 12, 13, 14, or 15, and each X independently is a recognition sequence (e.g., a recognition helix) capable of binding to 3 bp of a target sequence. In exemplary embodiments, n is 3, 6 or 9. In a particularly preferred embodiment, n is 6. In various embodiments, each X may independently have the same amino acid sequence or a different amino acid sequence as compared to other X sequences in the DNA binding domain. In an exemplary embodiment, each X is a sequence comprising 7 amino acids that has been designed to interact with 3 bp of the target binding site of interest using the Zinger Finger Design Tool from Scripps located on world wide web at scripps.edu/barbas/zfdesign/zfdesignhome.php. The engineered transcription factor optionally further comprises a VP64 transcription modulation domain. In some instances, the transcription factor may have a sequence at least 80%, at least 85%, at least 90%, at least 95%, at least 96%, at least 97%, at least 98%, or at least 99% sequence identity to any of SEQ ID NOs: 40-80.

The heterologous nucleic acid sequence optionally comprises a promoter to drive expression of the nucleic acid. A promoter can be native or non-native to the nucleic acid sequence to which it is operably linked, and native or non-native to a particular host cell. A promoter may be, in various aspects, a constitutive promoter, a tissue-specific promoter, or an inducible promoter. Examples of constitutive promoters include the Herpes Simplex virus (HSV), thymidine kinase (TK), Rous Sarcoma Virus (RSV), Simian Virus 40 (SV40), Mouse Mammary Tumor Virus (MMTV), Ad E1A, and cytomegalovirus (CMV) promoters. Additional examples of constitutive promoters include, a GAD2 promoter, a human synapsin promoter, CBA promoter, a minCMV promoter, a TATA box, a super core promoter, or an EF1a promoter. Examples of inducible promoters include, but are not limited to, those from genes such as cytochrome P450 genes, heat shock protein genes, metallothionein genes, and hormone-inducible genes, such as the estrogen gene promoter. Another example of an inducible promoter is the tet promoter that is responsive to tetracycline. In various embodiments, the heterologous nucleic acid comprises the CMV promoter. In certain embodiments, the heterologous nucleic acid comprises the Syn1 promoter.

Optionally, the heterologous nucleic acid comprises one more additional regulatory elements (optionally in addition to a promoter), such as, for example, sequences associated with transcription initiation or termination, enhancer sequences, and efficient RNA processing signals. Exemplary regulatory elements include, for example, an intron, an enhancer, UTR, stability element, WPRE sequence, a Kozak consensus sequence, posttranslational response element, a microRNA binding site, a polyadenylation (polyA) signal sequence, or a combination thereof. Regulatory elements can function to modulate gene expression at the transcriptional phase, post-transcriptional phase, or at the translational phase of gene expression. At the RNA level, regulation can occur at the level of translation (e.g., stability elements that stabilize mRNA for translation), RNA cleavage, RNA splicing, and/or transcriptional termination.

Regulatory elements included in the heterologous nucleic acid may be cell type selective regulatory elements, such as regulatory elements that drive expression in central nervous system cell types. Optionally, the regulatory element(s) selectively drive expression in GABAergic cells. GABAergic cells are inhibitory neurons which produce gamma-aminobutyric acid. GABAergic cells can be identified by the expression of glutamic acid decarboxylase 2 (GAD2). Other markers of GABAergic cells include GAD1, NKX2.1, DLX1, DLX5, SST, PV, and VIP. The regulatory element(s) may selectively drive expression in GABAergic cells that express parvalbumin (“PV cells”), to a greater degree than another cell type (e.g., another CNS cell type, such as a non-GABAergic neuron (such as non-PV GABAergic neurons)). Examples of non-PV CNS cells include excitatory neurons, dopaminergic neurons, astrocytes, microglia, motor neurons, and vascular cells. Non-GABAergic neurons also include cells that do not express one or more of GAD2, GAD1, NKX2.1, DLX1, DLX5, SST and VIP. In some cases, non-PV GABAergic neurons include, but are not limited to, calretinin (CR), somatostatin (SOM), cholecystokinin (CCK), CR+SOM, CR+neuropeptide Y (NPY), CR+vasointestinal polypeptide (VIP), SOM+NPY, SOM+VIP, VIP+choline acetyltransferase (ChAT), CCK+NPY, CR+SOM+NPY, and CR+SOM+VIP expressing cells.

In some aspects wherein the recombinant AAV vector comprises regulatory elements, the regulatory elements may comprise one or more sequences set forth SEQ ID NOs: 81-112. In some instances, the recombinant AAV vector comprises a heterologous nucleic acid comprising one or more of SEQ ID NOs: 81-112, (ii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NOs: 81-112, (iii) a functional fragment of any sequence of (i) or (ii), or (iv) a combination of any sequence of (i), (ii) and/or (iii). In some cases, sequence identity is measured by BLAST. A regulatory element may be located upstream or downstream of a transgene. Regulatory elements are further described in, e.g., International Patent Publication No. WO 2018/187363, incorporated herein by reference in its entirety and in particular with respect to regulatory elements and sequences.

In certain embodiments, the recombinant AAV vector comprises a nucleotide sequence operably linked to a regulatory element, wherein the regulatory element results in increased transgene expression by at least 2-fold as compared to expression of the transgene when operably linked to a CMV promoter. In certain embodiments, the promoter sequence produces at least 5-fold, 10-fold, 15-fold, 20-fold, 25-fold, 30-fold, 35-fold, 40-fold, 45-fold, 50-fold, 55-fold, 60-fold, 65-fold, 70-fold, or 75-fold, or at least 20-90 fold, 20-80 fold, 20-70 fold, 20-60 fold, 30-90 fold, 30-80 fold, 30-70 fold, 30-60 fold, 40-90 fold, 40-80 fold, 40-70 fold, 40-60 fold, 50-90 fold, 50-80 fold, 50-70 fold, 50-60 fold, 60-90 fold, 60-80 fold, 60-70 fold, 70-90 fold, 70-80 fold, or 80-90 fold greater expression of the transgene sequence in a mammalian cell relative to the level of expression of the same transgene sequence from the CMV promoter in the same type of mammalian cell. In certain embodiments, the promoter sequence drives expression of the transgene sequence in a high percentage of neuronal cells, e.g., at least 20%, 25%, 30%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or greater, or at least 20-90%, 20-80%, 20-70%, 30-90%, 30-80%, 30-70%, 40-90%, 40-80%, 40-70%, 50-90%, 50-80%, 50-70%, 60-90%, 60-80%, 60-70%, 70-90%, 70-80%, 80-100%, 80-95%, 80-90%, 90-100%, or 90-95% of GABAergic cells containing the vector express the transgene. In certain embodiments, the promoter sequence drives expression of the transgene in a high percentage of glial cells, e.g., at least 20%, 25%, 30%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or greater, or at least 20-90%, 20-80%, 20-70%, 30-90%, 30-80%, 30-70%, 40-90%, 40-80%, 40-70%, 50-90%, 50-80%, 50-70%, 60-90%, 60-80%, 60-70%, 70-90%, 70-80%, 80-100%, 80-95%, 80-90%, 90-100%, or 90-95% of oligodendrocytes containing the vector express the transgene.

In some aspects, an AAV expression cassette comprises a human-derived regulatory element of no more than 120 bp operably linked to a transgene of at least 3 kb, wherein the regulatory element results in increased transgene expression by at least 2-fold as compared to expression of the transgene when operably linked to a CMV promoter. In some cases, the increased transgene expression is at least 50-fold. In some cases, the increased transgene expression is at least 100-fold. In some cases, the increased transgene expression occurs in at least two different cell types (e.g., excitatory neurons and inhibitory neurons). In some cases, the increased transgene expression occurs in at least three different cell types (e.g., excitatory neurons, inhibitory neurons, and liver cells).

In some cases, such high expression of the transgene in a cell or in vivo is relative to expression of the transgene without said regulatory elements, wherein expression of the transgene with the regulatory elements is at least 1.5-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 50-fold, at least 100-fold, at least 150-fold, at least 200-fold, at least 250-fold, at least 300-fold, at least 400-fold, at least 500-fold, at least 600-fold, at least 700-fold, at least 800-fold, at least 900-fold, at least 1000-fold, at least 1010-fold, at least 1020-fold, at least 1030-fold, at least 1040-fold, or at least 1050-fold as compared to transgene expression without the regulatory elements, or as compared to transgene expression by a negative control (e.g., buffer alone, vector alone, or a vector comprising a sequence known to have no expression activity).

In certain embodiments, the heterologous nucleic acid further comprises a polyA signal sequence. Suitable polyA signal sequences include, for example, an artificial polyA that is about 75 bp in length (PA75) (see e.g., International Patent Publication No. WO 2018/126116), the bovine growth hormone polyA, SV40 early polyA signal, SV40 late polyA signal, rabbit beta globin polyA, HSV thymidine kinase polyA, protamine gene polyA, adenovirus 5 Elb polyA, growth hormone polyA, or a PBGD polyA. In exemplary embodiments, the polyA sequence is an hGH polyA (SEQ ID NO: 113) or a synthetic polyA (SEQ ID NO: 114). See also International Patent Publication No. WO 2019/109051, incorporated herein by reference. Typically, the polyA signal sequence is operably linked to a coding nucleic acid sequence.

The recombinant AAV vector may comprise inverted terminal repeats (ITRs), which may be derived from the same serotype as the capsid of the virus particle or derived from a different serotype (e.g., AAV2 ITRs and AAV9 capsid proteins; AAV2 ITRs and AAV8 capsid proteins; etc.). In a representative embodiment, the recombinant AAV vector comprises AAV2 ITRs.

The composition, in various aspects of the disclosure, displays properties suitable for direct administration to the central nervous system (e.g., direct administration to the brain or cerebrospinal fluid). In some embodiments, the composition demonstrates a conductivity of about 15.0 to about 17.0 mS/cm. “Conductivity” is the ability of an aqueous solution to conduct an electric current between two electrodes. Generally, electrical conductivity or specific conductivity is a measure the ability to conduct an electric current, which occurs via ion transport. As the amount of ions in a material increases, conductivity increases. Conductivity can be measured, for example, using a commercially available conductivity meter.

The disclosure further provides a method of delivering a heterologous nucleic acid of interest to a host cell, the method comprising administering the composition of the instant disclosure to a subject, e.g., a human subject. In this regard, the disclosure further provides a method of treating a medical condition in subject, such as a human subject, comprising administering the composition to a subject in need thereof. In various aspects, the medical condition is a neurological disorder or a neurodegenerative disorder. In some cases, the subject is suffering from (or at risk of suffering from) a psychiatric disorder, an autism spectrum disorder, epilepsy (e.g., Dravet syndrome), or neurodegeneration (e.g., neurodegeneration associated with Alzheimer's disease or Parkinson's disease). Examples of mutations include mutations in SCN1A.

Optionally, the composition is administered to a subject suffering from (or at risk of suffering from) a neurological condition associated with dysfunction of PV neurons. Disorders associated with dysfunctional PV neurons (such as disorders arising from loss of function mutations in SCN1A or Nav1.1) include, but are not limited to, Dravet syndrome, Ohtahara syndrome, epilepsy, early infantile epileptic encephalopathy 6 (EIEE6), familial febrile seizures 3A (FEB3A), intractable childhood epilepsy with generalized tonic-clonic seizures (ICEGTC), migraine, familial hemiplegic 3 (FHM3), Panayiotopoulos syndrome, familial atrial fibrillation 13 (ATFB 13), generalized epilepsy with febrile seizures plus type 1 (gefs+ type 1), Brugada syndrome, nonspecific cardiac conduction defect, generalized epilepsy with febrile seizures plus, benign familial infantile seizures, early infantile epileptic encephalopathy 11 (EIEE11), benign familial infantile epilepsy, neurodegeneration, tauopathies and Alzheimer's disease. In some Alzheimer's patients, production of amyloid β (Aβ) can affect the excitability of neurons, causing seizures and downregulation of the Nav1.1 sodium channel in PV neurons.

In some aspects of the disclosure, the neurological disorder is Dravet syndrome. A majority of Dravet syndrome cases is associated with mutations in the SCN1A and/or SCN2A genes. Mutations or abnormalities in SCN1A has also been associated with seizure disorders, epilepsy, autism, familial hemiplegic migraine type 3 (FHM3), genetic epilepsy with febrile seizures plus (GEFS+), and effectiveness of certain anti-seizure medications. For instance, ICS5N+5G>A mutation in SCN1A is associated with the maximum safe amount (dose) of the anti-seizure drugs phenytoin and carbamazepine. Symptoms associated with Dravet syndrome include seizures, memory defects, developmental delay, poor muscle tone and/or cognitive problems. Administration of the composition described herein can result in an improvement of one or more symptoms associated with any of the disorders described herein or preventing the development of or slowing the progression of one or more symptoms. With respect to Dravet syndrome, “treatment” includes, e.g., a reduction in number, duration, and/or intensity of seizures.

The composition provided herein may be administered to a subject via parenteral administration, subcutaneous administration, intravenous administration, intramuscular administration, intra-arterial administration, intraparenchymal administration, intrathecal administration, intra-cisterna magna administration, intracerebroventricular administration, or intraperitoneal administration. In various aspects, the composition is administered directly to the CNS (including directly to cerebral spinal fluid (CSF)), optionally by intraparenchymal injection, intrathecal injection, intra-cisterna magna injection, or intracerebroventricular injection. Methods of administering any of the compositions disclosed herein are discussed in greater detail below.

The present disclosure contemplates methods of administering a composition disclosed herein to a primate (e.g., a human), comprising intracerebroventricular (ICV) administration of the composition. Also described herein are compositions and methods for expressing a gene of interest or a biologically active variant and/or fragment thereof comprising administering to a primate a therapeutically effective amount of a composition comprising an adeno-associated virus vector encoding the gene of interest, wherein the route of administration is selected from the group consisting of intravenous administration, intrathecal administration, intracerebroventricular administration, intraparenchymal administration, or combinations thereof. Furthermore, described herein are compositions and methods to inhibit or treat one or more symptoms associated with a neuronal disease in a primate in need thereof, comprising administering a composition comprising an AAV to the primate, wherein the route of administration is selected from the group consisting of intravenous administration, intrathecal administration, intracerebroventricular administration, intraparenchymal administration, or combinations thereof.

In some embodiments, the disclosure provides for methods of administering a composition disclosed herein to a subject (e.g., a primate) via intrathecal administration or intracerebroventricular administration. The intrathecal space, into which the vector of the present invention is delivered in the case of intrathecal administration, is a space which is located around the spinal cord and filled with cerebrospinal fluid. This space is surrounded by a double-layer membrane consisting of arachnoid mater and dura mater. The intrathecal space is a space beneath the arachnoid mater, the inner layer of the double-layer membrane, and therefore, intrathecal administration means administration into the subarachnoid space. The space around the brain and the space around the spinal cord are both filled with CSF, and the cerebral ventricles in the brain are also filled with CSF. The cerebral ventricles, the pericerebral space and the intrathecal space are generally connected to form one continuous space, in which the CSF circulates. Therefore, intracerebroventricular administration and intrathecal administration are contemplated as being methods of administering any of the compositions disclosed herein to the CSF.

In some embodiments, the disclosure provides for methods of administering any of the compositions disclosed herein to a subject (e.g., a primate). In some aspects, the composition is delivered to the CNS. In some aspects, the composition is delivered to the cerebrospinal fluid. In some aspects, the composition is administered to the brain parenchyma. In some aspects, the composition is delivered to a primate by intracerebroventricular administration.

In some aspects, the composition is delivered to a subject (e.g., a primate) by intravenous administration. In some aspects, the composition is delivered to a subject (e.g., a primate) by intrathecal administration, e.g., intrathecal cisternal or intrathecal lumbar administration. In some aspects, the composition is delivered to the subarachnoid cistern, e.g., the cistema magna. In some aspects, the composition is delivered into the lumbar subarachnoid space surrounding the spinal nerves. In some aspects, the composition is delivered to a subject (e.g., a primate) by intraparenchymal administration. Broad distribution of compositions, described herein, within the central nervous system may be achieved with intraparenchymal administration, intrathecal administration, or intracerebroventricular administration.

In some aspects, any of the compositions disclosed herein is administered to a subject (e.g., a primate) in combination with a contrast agent, e.g., gadolinium or gadoteridol. In other aspects, the vector is not administered in combination with a contrast agent, e.g., gadolinium or gadoteridol.

In some aspects, any of the compositions disclosed herein is administered via intracerebroventricular (ICV) administration to any one or more ventricles of the brain. In some aspects, the composition is administered via ICV administration unilaterally into one ventricle, e.g., into the left lateral ventricle or right lateral ventricle. In some aspects, the composition is administered via ICV administration unilaterally into the left lateral ventricle. In some embodiments, the composition is administered via ICV administration unilaterally into the right lateral ventricle. In some aspects, the composition is aspects via ICV administration bilaterally, e.g., into the left and right lateral ventricle. In some embodiments, the composition is administered via ICV administration to one ventricle of the brain, e.g., into only the left ventricle. In some aspects, the composition is administered via ICV administration to only the left lateral ventricle. In some aspects, the composition is administered via ICV administration to only the right lateral ventricle. In some aspects, the composition is administered via ICV administration to only the third ventricle. In some aspects, the composition is administered via ICV administration to only the fourth ventricle. In some aspects, the composition is administered via ICV administration to more than one ventricle of the brain, e.g., into the left ventricle, right ventricle, and third ventricle. In some aspects, the composition is administered via ICV administration simultaneously, e.g., into the left ventricle and right ventricle at the same time point. In some aspects, the composition is administered via ICV administration sequentially, e.g., into the left ventricle and right ventricle at different time points. In some aspects, each dose of the composition is administered via ICV administration at least 24 hours apart.

In some aspects, the disclosure provides a method of administering a composition to a primate, comprising intracerebroventricular (ICV) administration of a composition to the primate, wherein the composition comprises a vector capable of expressing a transgene, and wherein ICV administration results in increased transgene expression in the central nervous system (CNS) by at least 1.25-fold as compared to expression of the transgene when the composition is administered by any other route of administration. In certain aspects, ICV administration produces at least 1.5-fold, 1.75-fold, 2-fold, 3-fold 5-fold, 10-fold, 15-fold, 20-fold, 25-fold, 30-fold, 35-fold, 40-fold, 45-fold, 50-fold, 55-fold, 60-fold, 65-fold, 70-fold, or 75-fold, or at least 20-90 fold, 20-80 fold, 20-70 fold, 20-60 fold, 30-90 fold, 30-80 fold, 30-70 fold, 30-60 fold, 40-90 fold, 40-80 fold, 40-70 fold, 40-60 fold, 50-90 fold, 50-80 fold, 50-70 fold, 50-60 fold, 60-90 fold, 60-80 fold, 60-70 fold, 70-90 fold, 70-80 fold, 80-90 fold greater expression of the transgene sequence in the central nervous system (CNS) as compared to expression of the transgene when the composition is administered by any other route of administration. In some embodiments, ICV administration results in gene transfer throughout the brain. In certain embodiments, the gene transfer occurs in the frontal cortex, parietal cortex, temporal cortex, hippocampus, medulla, and occipital cortex. In certain embodiments, the gene transfer is dose dependent.

In certain aspects, the composition comprises a vector which further comprises a cell-type selective regulatory element. In certain aspects, the regulatory element is selectively expressed in the brain. In certain aspects, the regulatory element is selectively expressed in the frontal cortex, parietal cortex, temporal cortex, hippocampus, medulla, and occipital cortex. In certain embodiments, the regulatory element is selectively expressed in the spine. In certain aspects, the regulatory element is selectively expressed in the spinal cord and dorsal root ganglion. In certain aspects, the regulatory element is selectively expressed in neuronal cells. In certain aspects, the neuronal cells are selected from the group consisting of unipolar, bipolar, multipolar, or pseudounipolar neurons. In certain aspects, the neuronal cells are GABAergic neurons. In certain aspects, the regulatory element is selectively expressed in glial cells. In certain aspects, the glial cells are selected from the group consisting of astrocytes, oligodendrocytes, ependymal cells, Schwann cells, and satellite cells. In certain aspects, the regulatory element is selectively expressed in non-neuronal cells.

The disclosure contemplates a method of administering the composition disclosed herein by multiple routes of administration to a subject (e.g., a primate). For example, the disclosure provides for methods of administering the composition disclosed herein by one route of administration (e.g., intracerebroventricular administration) and the same composition also by another route of administration (e.g., intravenous administration). The disclosure further provides for methods of administering the composition disclosed herein by intracerebroventricular administration and the same composition also by intravenous administration. In some aspects, the disclosure provides for methods of administering the composition disclosed herein by intrathecal administration and the same composition also by intravenous administration. In some aspects, the disclosure provides for methods of administering the composition disclosed herein by one route of administration (e.g., intracerebroventricular administration) and an additional therapeutic agent (e.g., any of the additional therapeutic agents disclosed herein) by another route of administration (e.g., intravenous administration). In some aspects, the disclosure provides for methods of administering the composition disclosed herein by intracerebroventricular administration and an additional therapeutic agent by intravenous administration. In some aspects, the disclosure provides for methods of administering the composition disclosed herein by intrathecal administration and an additional therapeutic agent by intravenous administration. In some aspects, the disclosure provides for methods of administering the composition disclosed herein by intravenous administration and an additional therapeutic agent by intracerebroventricular administration. In some aspects, the disclosure provides for methods of administering the composition disclosed herein by intravenous administration and an additional therapeutic agent by intrathecal administration. In some aspects, the intrathecal administration comprises an intrathecal cisternal administration. In some aspects, the intrathecal administration comprises an intrathecal lumbar administration. In some aspects, the route of administration is any one or combination of intravenous administration, intrathecal administration, intracerebroventricular administration, or intraparenchymal administration.

In some aspects, the route of administration is any one or combination of subcutaneous administration, intramuscular administration, intraarterial administration, intraperitoneal administration, or intracranial administration.

In some aspects, the administration comprises administration through an injection. In some aspects, the administration comprises administration through a cannula. In some aspects, the composition is administered as a bolus, e.g., as a single injection. In some embodiments, the composition is administered continuously, e.g., an infusion using a syringe pump.

In some aspects, intracerebroventricular (ICV) administration comprises inserting a cannula through a hole in the skull, through the brain tissue, into a CSF-filled ventricle of the brain. In some embodiments, a single cannula is inserted (e.g., into either of the two lateral ventricles). In some aspects, two cannulas may be inserted (into both lateral ventricles). In some aspects, the cannula may be connected to a syringe or infusion pump for one-time administration, or a controlled device, such as an Ommaya reservoir. In some aspects, the disclosure provides for administration of any of the vectors disclosed herein to one or more lateral ventricles of a subject. Because of the concern for neurovascular injury and intracranial hemorrhage, repeated “tips” of the ventricle are not routinely performed. An exception to this rule might be in premature neonates who during pathologic conditions often have very large ventricles, a thin cortical mantle, and an open fontanelle, making the cumulative risks of repeated tips lower in this population.

Intrathecal intracisternal infusions are less frequently performed in humans due to the proximity of the cisterns to vital brain tissues. However, in some embodiments, intrathecal infusion devices (e.g., Medtronic devices) can be inserted in the lumbar subarachnoid space and a catheter extended upwards toward the cranium for administration. In some aspects, intrathecal administration to a human being comprises surgically inserting a catheter at about the L4/L5 interspace and administering either (i) a bolus dose (via syringe or Ommaya reservoir), (ii) a short term infusion (via a pump), or (iii) a long term infusion (via an implantable programmable pump system, e.g., Synchromed II, Medtronic, where the pump is placed in a subcutaneous pocket somewhere in the body such as the abdominal region). See, e.g., Hamza M, et al. Neuromodulation, 2015; 18(7):636-48).

In some aspects, intrathecal administration of any of the compositions disclosed herein comprises administering the composition into the lumbar cistern by means of a lumbar puncture. In some aspects, a spinal tip can be performed at the bedside with local anesthetic under sterile conditions. In some aspects, a spinal needle is advanced into the thecal sac through an interlaminar space in the lower lumbar spine. In some embodiments, access into the lumbar cistern is confirmed when CSF is obtained. See, e.g., Cook A M, et al. Pharmacotherapy. 2009; 29(7):832-45.

In some aspects, the composition disclosed herein is administered to a subject (e.g., a primate) by injecting the compositions through a spinal needle. This technique is used frequently for administration of chemotherapeutic drugs. Advantages of this technique may include its relatively low risk and ability to be performed at the bedside under local anesthetic. A disadvantage of this technique is that a separate puncture must be performed each time a dose is given, resulting in a cumulative risk of introducing infection, developing a cutaneous-CSF fistula, injuring nerve roots, and causing intraspinal hemorrhage. In some embodiments, to circumvent this problem, a temporary indwelling catheter can be placed by using a similar technique with a larger Touhy needle.

In some aspects, the compositions disclosed herein may be administered to a subject (e.g., a primate) by advancing a catheter into the thecal sac of the subject through the center of the needle, wherein the needle is subsequently withdrawn. In some aspects, the catheter is then tunneled subcutaneously through the skin where it can be accessed sterilely for scheduled doses of a chosen intrathecal drug. Disadvantages of this technique include the risk of infection with prolonged catheter placement and catheter malfunction from occlusion, kinking, or displacement. However, these disadvantages may be mitigated by removing or replacing the catheter after a few days (e.g., 1-4 days).

In some aspects, the composition disclosed herein is administered via a catheter-based device. In some aspects, a permanent catheter-based device is implanted. In some aspects, a temporary catheter-based device is implanted. In some aspects, for permanent access, a catheter that is connected to a subcutaneous reservoir (e.g., an Ommaya reservoir) is implanted. In some aspects, the catheter is connected to the Ommaya reservoir. The Ommaya reservoir can be accessed repeatedly at the bedside with a sterile puncture through the scalp into the reservoir by using a 25-gauge needle. In some aspects, a few milliliters of CSF is withdrawn before injecting the therapeutic agent. Contamination and infection of the Ommaya reservoir is a risk, although less likely than with other methods of accessing the intraventricular compartment (approximately 10% of patients ultimately have CSF contaminated with bacteria). Infection rates often appear higher in case series reporting infectious complications with Ommaya reservoirs because of the duration of implantation (often >1 yr) compared with other more temporary access devices. Other rare complications that may occur with Ommaya reservoirs include leukoencephalopathy, white matter necrosis, and intracerebral hemorrhage.

In situations that require limited access to the CSF space, a ventriculostomy can be placed. With this technique, the catheter is tunneled under the skin away from the burr hole. The catheter is usually connected to a sterile collection chamber. The catheter can be accessed sterilely as needed for administration of any of the vectors disclosed herein. In some aspects, a composition described herein may be administered by injecting the solution into the most proximal port of the ventriculostomy and flushing the solution into the brain with a small amount of normal saline (3-5 ml). After this instillation, the ventriculostomy tubing is typically clamped for at least 15 minutes to allow for the injected solution to equilibrate in the CSF before reopening the drain. Patients with persistently elevated intracranial pressure may not tolerate the abrupt cessation of CSF drainage, so ventriculostomy clamping should be done with caution and close monitoring of the patient. A ventriculostomy is ideal for a condition that requires a limited time period for CSF drainage or intraventricular administration of any of the vectors disclosed herein.

In some aspects, the disclosure provides for methods of administering the composition disclosed herein to a subject, wherein the subject is a primate. In some aspects, the primate is a human. In some aspects, the primate is a non-human primate. In some aspects, the non-human primate is an old world monkey, an orangutan, a gorilla, a chimpanzee, a crab-eating macaque, a rhesus macaque, or a pig-tailed macaque.

The present disclosure contemplates methods of treating a subject (e.g., a primate such as a human or a cynomolgus monkey) in need thereof, comprising administering to the subject any of the nucleic acids, vectors, viral particles, and/or compositions disclosed herein.

In certain embodiments, a composition provided herein comprises an “effective amount” or a “therapeutically effective amount” of active agent (e.g., rAAV). As used herein, such amounts refer to an amount effective, at dosages and for periods of time necessary to achieve the desired therapeutic result.

The dosage of the composition of the disclosure depends on factors including the route of administration, the disease to be treated, and physical characteristics (e.g., age, weight, general health) of the subject. Dosage may be adjusted to provide a desired therapeutic response. Typically, a dosage may be an amount that effectively treats the disease without inducing significant toxicity. In one embodiment, an AAV vector provided herein can be administered to the patient for the treatment of a neuronal disease (including for example, Dravet syndrome) in an amount or dose within a range of 5×10¹⁰ to 1×10¹⁴ gc/kg (genome copies per kilogram of patient body weight (gc/kg)). In a more particular aspect, the AAV vector is administered in an amount comprised within a range of about 5×10¹⁰ gc/kg to about 1×10¹³ gc/kg, or about 1×10¹¹ to about 1×10¹⁵ gc/kg, or about 1×10¹¹ to about 1×10¹⁴ gc/kg, or about 1×10¹¹ to about 1×10¹³ gc/kg, or about 1×10¹¹ to about 1×10¹² gc/kg, or about 1×10¹² to about 1×10¹⁴ gc/kg, or about 1×10¹² to about 1×10¹³ gc/kg, or about 5×10¹¹ gc/kg, 1×10¹² gc/kg, 1.5×10¹² gc/kg, 2.0×10¹² gc/kg, 2.5×10¹² gc/kg, 3×10¹² gc/kg, 3.5×10¹² gc/kg, 4×10¹² gc/kg, 4.5×10¹² gc/kg, 5×10¹² gc/kg, 5.5×10¹² gc/kg, 6×10¹² gc/kg, 6.5×10¹² gc/kg, 7×10¹² gc/kg, 7.5×10¹² gc/kg, 8×10¹² gc/kg, 8.5×10¹² gc/kg, 9×10¹² gc/kg, 9.5×10¹² gc/kg, Ix 10¹³ gc/kg, 1.5×10¹³ gc/kg, 2.0×10¹³ gc/kg, 2.5×10¹³ gc/kg, 3×10¹³ gc/kg, 3.5×10¹³ gc/kg, 4×10¹³ gc/kg, 4.5×10¹³ gc/kg, 5×10¹³ gc/kg, 5.5×10¹³ gc/kg, 6×10¹³ gc/kg, 6.5×10¹³ gc/kg, 7×10¹³ gc/kg, 7.5×10¹³ gc/kg, 8×10¹³ gc/kg, 8.5×10¹³ gc/kg, 9×10¹³ gc/kg, or 9.5×10¹³ gc/kg. In another aspect, an AAV vector as provided herein can be administered to the patient for the treatment of a neuronal disease (including for example, Dravet syndrome) in an amount or dose depending on the volume of CSF of the patient. An AAV vector provided herein (e.g., provided in the disclosed composition) may be administered to a patient at an amount or dose within a range of 5×10¹¹ to 1×10¹² gc/ml of estimated CSF volume (genome copies per ml of estimated CSF volume of patient (gc/ml)). In a more particular embodiment, the AAV vector is administered in an amount comprised within a range of about 5×10¹⁰ gc/ml to about 1×10¹³ gc/ml, or about 1×10¹¹ to about 1×10¹⁵ gc/ml, or about 1×10¹¹ to about 1×10¹⁴ gc/ml, or about 1×10¹¹ to about 1×10¹³ gc/ml, or about 1×10¹¹ to about 1×10¹² gc/ml, or about 1×10¹¹ to about 1×10¹⁴ gc/ml, or about 1×10¹¹ to about 1×10¹³ gc/ml, or about 5×10¹¹ gc/ml to about 1×10¹² gc/ml, or about 3×10¹¹ gc/ml to about 2.0×10¹² gc/ml, or about 2.5×10¹¹ gc/ml to about 3×10¹² gc/ml. In some aspects, an AAV provided herein may be administered in an amount of about 5×10¹¹ gc/ml, or about 1×10¹² gc/ml. The gc/kg or gc/ml may be determined, for example, by qPCR or digital droplet PCR (ddPCR) (see e.g., M. Lock et al, Hum Gene Ther Methods. 2014 April; 25(2):115-25). In another aspect, a composition comprising an AAV vector provided herein can be administered to the patient for the treatment of a neuronal disease (including for example, Dravet syndrome) in an amount or dose within a range of 1×10⁹ to 1×10¹¹ iu/kg (infective units of the vector (iu)/subject's or patient's body weight (kg)). In certain aspects, the composition may be formed in a unit dose as needed. Such single dosage units may contain about 1×10⁹ gc to about 1×10¹⁶ gc, about 1×10¹⁰ gc to about 1×10¹⁵ gc, about 1×10² gc to about 1×10¹⁵ gc, about 5×10¹³ gc to about 5×10¹⁴ gc, about 5×10¹³ gc to about 1.4×10¹⁴ gc, about 9×10¹³ gc to about 2×10¹⁴ gc, or about 1×10¹⁴ gc to about 1.5×10¹⁴ gc.

Compositions of the disclosure may be administered to a subject in need thereof, for example, one or more times (e.g., 1-10 times or more) daily, weekly, monthly, biannually, annually, or as medically necessary. In an exemplary embodiment, a single administration is sufficient. The composition, in various aspects of the disclosure, is suitable for use in human subjects and is administered by intracerebroventricular administration. In various aspects, the composition is suitable for use in human subjects and is administered by intracerebroventricular administration, intravenous administration, intrathecal administration, intraparenchymal administration, or combinations thereof. In various aspects, the composition is delivered via a peripheral vein by bolus injection. In various aspects, the composition is delivered via a peripheral vein by infusion over about 10 minutes (±5 minutes), over about 20 minutes (±5 minutes), over about 30 minutes (±5 minutes), over about 60 minutes (±5 minutes), or over about 90 minutes (±10 minutes). In various aspects, the composition is delivered to the CSF by bolus injection. In various aspects, the composition is delivered to the CSF by infusion over about 10 minutes (±5 minutes), over about 20 minutes (±5 minutes), over about 30 minutes (±5 minutes), over about 60 minutes (±5 minutes), or over about 90 minutes (±10 minutes).

The disclosure further provides a kit comprising the composition described herein and instructions for use. The composition is, in various aspects of the disclosure, provided as a sterile composition for administration to the subject. In this regard, the composition may be a “pharmaceutical composition,” i.e., a composition suitable for administration to a subject, such as a human.

Optionally, the composition is present in a delivery device, a container for storage or shipment or administration, or a container suitable for use in drug substance or drug product manufacturing. The kit may comprise a container (e.g., vial, syringe, or infusion bag) which is a single-use container (i.e., a container that holds one dose formulation plus enough extra to ensure that a full single dose can be administered to a patient from the container, but not so much extra that the container could be used to administer a second dose) or a multiple-use container. The container may be a drug delivery device (e.g., syringe) or container for storage or shipment or administration (e.g., a vial or bag).

Alternatively, the kit may comprise one or more containers comprising the composition and instructions for use in manufacturing or preparing a drug substance or a drug product. Indeed, the composition may be used at any step in the manufacturing process prior to loading into a final storage container (e.g., vial, syringe, or infusion bag). The disclosure contemplates a container suitable for use in manufacturing prior to loading into a final container intended for distribution, wherein the container comprises a composition comprising recombinant adeno-associated virus (AAV) vectors comprising a heterologous nucleic acid, sodium chloride, potassium chloride, magnesium chloride, phosphate buffer, and a non-ionic surfactant, at pH of 7.2-7.4 (as described herein). For example, the kit may contain one or more containers suitable for use in a filtration system or system for filling parts of a drug delivery system (e.g., vials, syringes, or infusion bags), wherein the container comprises the composition described herein. The kit described herein may include separate containers comprising one or more of the composition components (rAAV vectors, sodium chloride, potassium chloride, magnesium chloride, phosphate buffer, and/or a non-ionic surfactant). For example, the kit may comprise a container comprising sodium chloride, potassium chloride, magnesium chloride, phosphate buffer, and a non-ionic surfactant and a separate container comprising the rAAV.

The disclosure further contemplates a method of making a pharmaceutical composition, the method comprising combining (i) an admixture comprising (a) sodium chloride, optionally about 145 mM to about 150 mM sodium chloride; (b) potassium chloride, optionally about 1.5 mM to about 4.5 mM potassium chloride; (c) magnesium chloride, optionally about 0.05 mM to about 1 mM magnesium chloride; (d) phosphate buffer (e.g., sodium phosphate), optionally present in an amount sufficient to provide about 0.5 mM to about 2 mM (e.g., about 1 mM) phosphate; and (e) a non-ionic surfactant (e.g., a poloxamer, such as poloxamer 188), optionally present at a concentration in the range of about 0.001% to about 0.01% (w/V); with (ii) rAAV vectors. Optionally, the resulting pharmaceutical composition comprises about 5×10¹³ vg/mL to about 1×10¹⁴ vg/mL (e.g., about 8×10¹³ vg/mL) of the recombinant AAV vectors. Also optionally, the resulting pharmaceutical composition comprises a pH of 7.2-7.4.

In general, “sequence identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity to a reference sequence (e.g., nucleic acid or amino acid sequence) may be calculated as the number of exact matches between two optimally aligned sequences divided by the length of the reference sequence and multiplied by 100. Conservative substitutions are not considered as matches when determining the number of matches for sequence identity. It will be appreciated that where the length of a first sequence (A) is not equal to the length of a second sequence (B), the percent identity of A:B sequence will be different than the percent identity of B: A sequence. Sequence alignments, such as for the purpose of assessing percent identity, may be performed by any suitable alignment algorithm or program, including but not limited to the Needleman-Wunsch algorithm (see, e.g., the EMBOSS Needle aligner available on the world wide web at ebi.ac.uk/Tools/psa/emboss_needle/), the BLAST algorithm (see, e.g., the BLAST alignment tool available on the world wide web at blast.ncbi.nlm.nih.gov/Blast.cgi), the Smith-Waterman algorithm (see, e.g., the EMBOSS Water aligner available on the world wide web at ebi.ac.uk/Tools/psa/emboss_water/), and Clustal Omega alignment program (see e.g., the world wide web at clustal.org/omega/and F. Sievers et al., Mol Sys Biol. 7:539 (2011)). Optimal alignment may be assessed using any suitable parameters of a chosen algorithm, including default parameters. The BLAST program is based on the alignment method of Karlin and Ahschul, Proc. Natl. Acad. Sci. USA 87:2264-2268 (1990) and as discussed in Ahschul, et al., J. Mol. Biol. 215:403-410 (1990); Karlin and Ahschul, Proc. Natl. Acad. Sci. USA 90:5873-5877 (1993); and Ahschul et al., Nucleic Acids Res. 25:3389-3402 (1997).

### EXAMPLES

The following examples are given merely to illustrate the present invention and not in any way to limit its scope.

### Example 1: Recombinant AAV Sample Preparation

Recombinant AAV (rAAV) for the studies described herein were produced in HEK-293 T cells by co-transfection of three plasmids: (i) a vector expressing AAV Rep and Cap genes, (ii) pALD-X80 Adenovirus helper plasmid (Aldevron), and (iii) a plasmid containing the AAV genome to be packaged. The transgene encoded in the AAV genome plasmid is an engineered transcriptional activator (eTF) that includes a DNA binding domain with known DNA binding specificity (described in International Patent Publication No. WO 2019/109051 entitled “Engineered DNA Binding Proteins,” incorporated herein by reference in its entirety). The rAAV capsid serotype is rAAV9. After culture post-transfection, the cells and supernatant were harvested and processed for rAAV concentration and diafiltration using a Millipore-Sigma Amicon Ultra-15 Centrifugal Unit (Millipore-Sigma P/N UFC910024) with a 100 kilo-Dalton (kD) molecular weight cut-off (MWCO) at 3000 xg. The viral genomes/mL (vg/mL) of each concentrated sample was approximated using UV absorbance. Buffer exchange was performed on the concentrated samples by diafiltration (approximately 10×), after which the samples were sterile filtered. The final concentration was re-confirmed by UV and subsequently tested via ddPCR as set forth in Lock, et al. (Hum. Gene Ther. 2010; 21:1273-1285) using forward and reverse PCR primers and a hybridization probe specific for the packaged rAAV genome.

### Example 2: Assays

A series of assays were employed to evaluate rAAV stability in multiple sample formulations at time 0 (t=0) and across multiple stress conditions. The assays and the attribute(s) they are designed to measure are provided below.

### Size Exclusion Chromatography

Size exclusion chromatography (SEC or SE-HPLC) is a liquid chromatography technique that separates species in samples based on molecular size. The rAAV samples were analyzed under non-dissociating conditions by injection onto a Sepax SRT SEC-1000 column using a mobile phase of 2×DPBS and 10% ethanol. The species were eluted in order of decreasing molecular size (i.e., the species are eluted from largest to smallest). The species eluted from the column were analyzed at two wavelengths: 260 nm to detect the rAAV genomic DNA (vg) and 280 nm to detect rAAV capsid (cp). SEC is able to detect intact rAAV monomers as well as species that are larger or smaller than rAAV monomers. These are generically referred to as “high molecular weight species” (HMWS) and “low molecular weight species” (LMWS). HMWS include aggregates of rAAV/rAAV components and LMWS include empty rAAV capsids, non-encapsidated viral genomes, and subparts thereof. HMWS and LMWS are considered to be non-functional in this assay and thus represent undesirable species in a sample. SEC thus provides a sensitive method to monitor viral stability under different stress conditions.

### Reverse Phase HLPC

Reverse Phase HLPC (RP-HPLC) is a liquid chromatography technique that dissociates and separates proteins in order of increasing hydrophobicity. Samples were by injection on a C3 column in an acetonitrile gradient in the presence of an ion-pairing agent, trifluoroacetic acid (TFA, 0.2%). The relative concentration of the rAAV capsid proteins VP1, VP2, and VP3 were quantitated in each sample using the areas under the curve at 280 nm UV detection.

### Polydispersity and Extrinsic DNA

The UNcle system from UNchained Labs was used to assess the polydispersity of particles in a sample (i.e., the distribution of particle sizes in a sample) as well as the amount of extrinsic DNA (i.e., vg that are not packaged in an rAAV capsid). Certain assays were performed during thermal transition (e.g., from 15° C. to 95° C.) or at specific hold temperatures as indicated.

The UNcle system uses dynamic light scattering (DLS) to measure the polydispersity of particles in a sample, which describes how much material there is present of the different size “slices” in the sample. In DLS, the native distribution is the intensity distribution which indicates how much light is scattered from the various size “slices” or “bins.” The mean size and the standard deviation from that mean can be obtained directly from the statistics of the distribution. The (absolute) standard deviation (or “halfwidth”) of the distribution can be compared to the mean, and a relative polydispersity (standard deviation/mean) can be obtained. Historically, instead of requiring a distribution, a simpler forced single exponential fitting scheme (the cumulant method) has been used to find an overall mean size (by intensity) and an overall polydispersity (the normalized second cumulant). For a theoretical Gaussian distribution, the overall polydispersity would be the relative polydispersity of the distribution. Traditionally, this overall polydispersity has also been converted into an overall polydispersity index PDI which is the square of the light scattering polydispersity. For a perfectly uniform sample, the PDI would be 0.0. A PDI of ≤0.1 is generally considered monodispersed, a PDI of 0.1-0.4 is considered moderately polydispersed, and a PDI of >0.4 is considered broadly polydispersed.

Extrinsic fluorescence in the presence of Sybr Gold measures the level of external non-encapsidated DNA in a sample, which can be in the form of unpackaged DNA present in the sample (DNA that co-purified with the rAAV during the manufacturing process) as well as packaged DNA released from rAAV capsids in the sample, e.g., during storage, hold and/or stress conditions.

### Example 3: Sample Analyses

In this example, thermal stresses were applied to different rAAV sample formulations followed by analyses that monitored several physical and biochemical characteristics indicative of rAAV stability (described in Example 2).

Analyses were performed on 11 different rAAV sample formulations (Table 1), all stored ≤−70° C. Samples were subjected to different stress conditions and analyzed using one or more of the assays described in Example 2. Stress conditions included freeze/thaw (FT) cycles (from ≤−70° C. to room temperature (RT) for indicated number of times) and incubation at RT or 37° C. for the indicated number of days. As shown in Table 1, the sample formulations differed with respect to the base buffer and the presence/amount of three different components: Kolliphor® P 188 (BASF), MgCl₂, and trehalose. Samples 10 and 11 had higher AAV titers that samples 1-9.

TABLE 1
Sample formulations

| | AAV Titer | Base | P188 | MgCl₂ | Trehalose |
|---|---|---|---|---|---|
| Sample | (vg/mL) | Buffer | (%) | (mM) | (%) |
| 1 | 8.0 × 10¹³ | Buffer 1 | 0.001 | — | — |
| 2 | 8.0 × 10¹³ | Buffer 1 | 0.005 | — | — |
| 3 | 8.0 × 10¹³ | Buffer 2 | 0.001 | — | — |
| 4 | 8.0 × 10¹³ | Buffer 2 | 0.005 | — | — |
| 5 | 8.0 × 10¹³ | Buffer 2 | 0.005 | 0.8 | — |
| 6 | 8.0 × 10¹³ | Buffer 2 | 0.005 | 0.8 | 0.05 |
| 7 | 8.0 × 10¹³ | Buffer 2 | 0.005 | — | 0.05 |
| 8 | 8.0 × 10¹³ | Buffer 3 | 0.005 | — | — |
| 9 | 8.0 × 10¹³ | Buffer 3 | 0.005 | 0.8 | — |
| 10 | 1.20 × 10¹⁴ | Buffer 3 | 0.005 | 0.8 | 0.05 |
| 11 | 1.20 × 10¹⁴ | Buffer 3 | 0.005 | — | 0.05 |

Buffer 1: 137 mM NaCl, 2.7 mM KCl, 8 mM Na₂HPO₄, and 2 mM KH₂PO₄; pH 7.3
Buffer 2: 1 mM Phosphate [0.145 mM NaH₂PO₄ * H₂0 and 0.854 mM Na₂HPO₄ * 7H₂0], 148 mM NaCl, 3 mM KCl; pH 7.3
Buffer 3: 20 mM Tris; pH 7.3
P188: Kolliphor ® P 188 (BASF) [poly(ethylene glycol)-block-poly(propylene glycol)-block-poly(ethylene glycol)]

Assessment for the presence of the viral genome in each sample by ddPCR analysis showed no discernable difference between any of the samples at t=0 or under any of the stress conditions, indicating that the genomic DNA was not degraded (data not shown). It is noted that this assay does not provide any information with respect to the infectivity of the rAAV present in the sample or whether the genomic DNA is encapsidated.

### Size Exclusion Chromatography

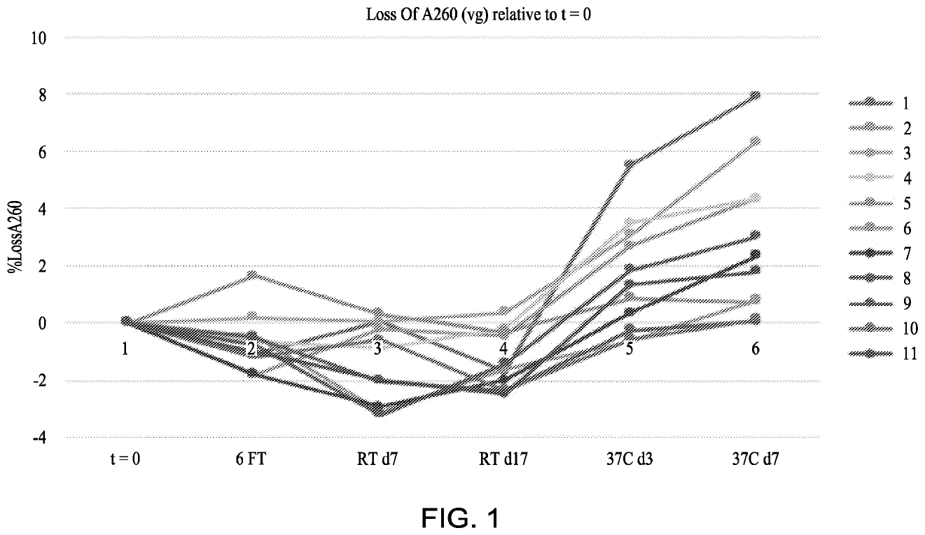

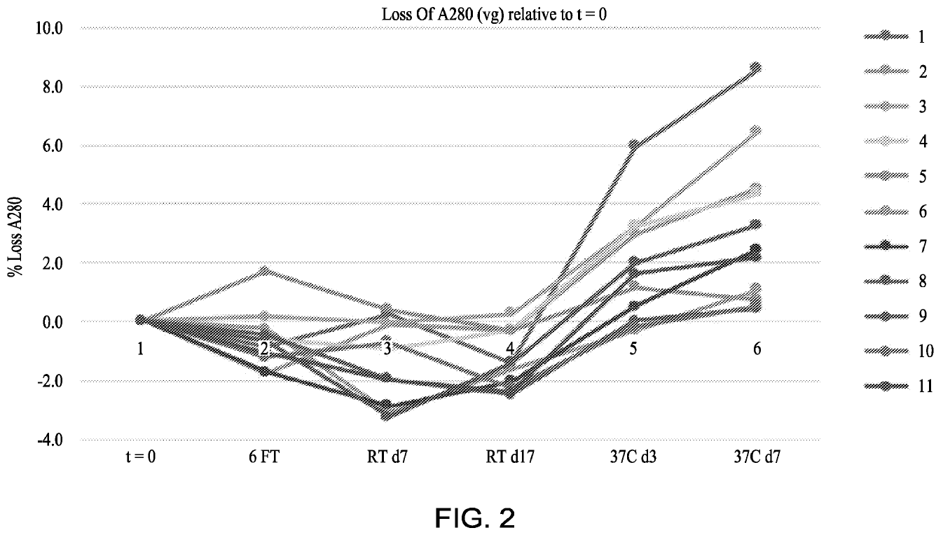

Results of the SEC analysis are shown in FIGS. 1 and 2. As seen in these figures, the percent loss of vg, represented by a loss of absorbance at 260 nm (FIG. 1) and cp, represented by a loss of absorbance at 280 nm (FIG. 2) was most significant in samples 1 and 3 when stressed at 37° C. Both of these samples include the lower amount of P188 (0.001%).

Sample 1, a Buffer 1-based formulation, showed the most instability, with a 5.5% and 7.9% loss of vg on days 3 and 7 at 37° C., respectively, and a 6.0% and 8.6% loss of cp at days 3 and 7 at 37° C., respectively.

Sample 3, a Buffer 2-based sample, performed slightly better than sample 1, showing a 6.5% loss of vg and cp on day 7 at 37° C.

Samples 5, 9, 6, and 10 showed the highest stability of the samples tested in this assay with the least vg and cp loss. Samples 5 and 9 included MgCl₂, while samples 6 and 10 included both MgCl₂ and trehalose. The 6×FT stress condition as well as the 7 day incubation at RT showed little to no appreciable impact to cp or vg loss in any of the samples tested.

### Reverse Phase HLPC

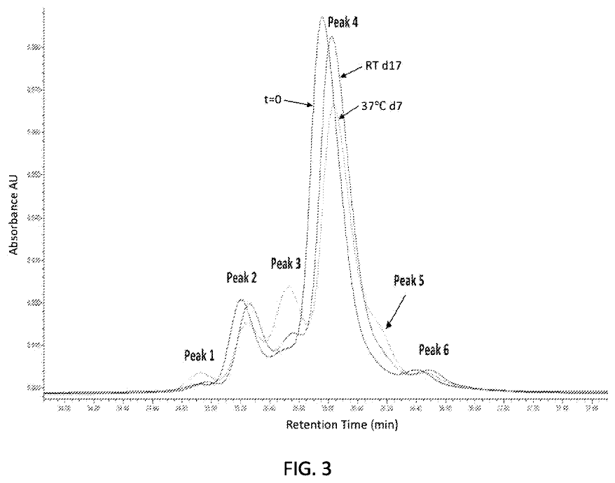

FIG. 3 shows RP-HPLC results of a representative rAAV sample at t=0, incubation at RT for 17 days, and incubation at 37° C. for 7 days. At t=0 and 17 days of incubation at RT, five peaks (P1-P4 and P6) are observed. At 7 days of incubation at 37° C., a higher stress condition, a sixth peak (P5) becomes apparent (back shoulder off P4 in FIG. 3). This peak was shown to increase in magnitude as a function of time (data not shown). P1 and P3 also increase under thermal stress conditions while P2, P4, and P6 decrease. P2 and P3 are in near equimolar quantities and thus likely represent VP1 and VP2, while P4, the largest and highest quantity, likely represents VP3 (given their expected ratios in AAV capsids). The apparent increase in P1 and P3 under thermal stress conditions may represent a modification of VP1 and VP2 (e.g., a deamidated and/or oxidated form) that co-elutes with its non-modified counterpart.

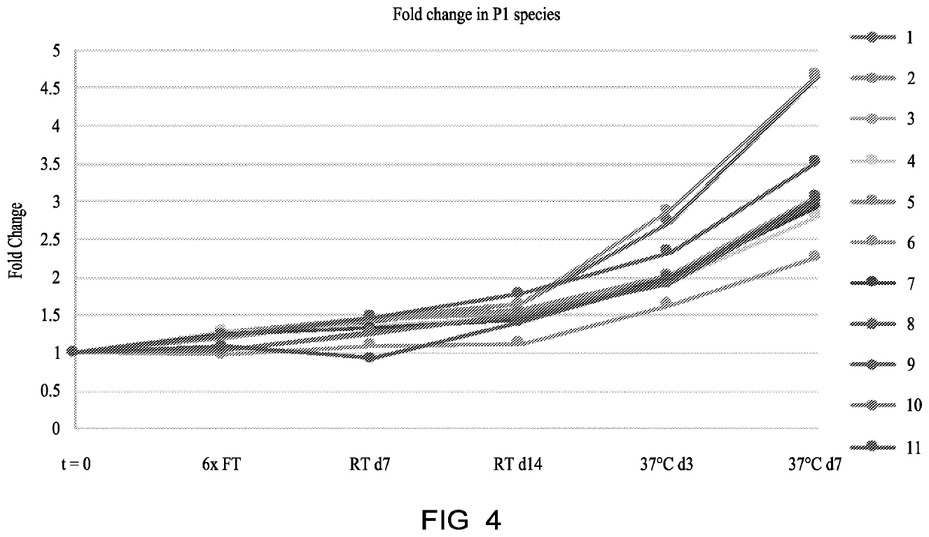

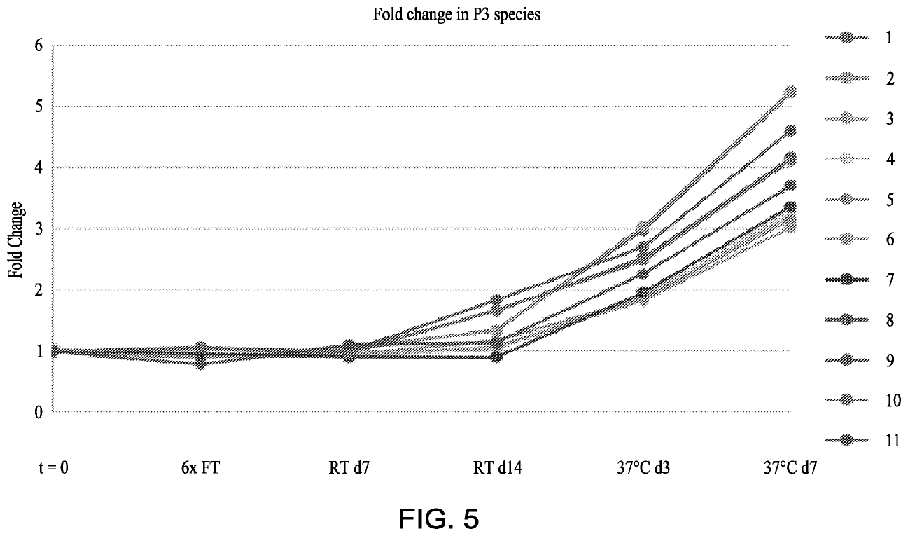

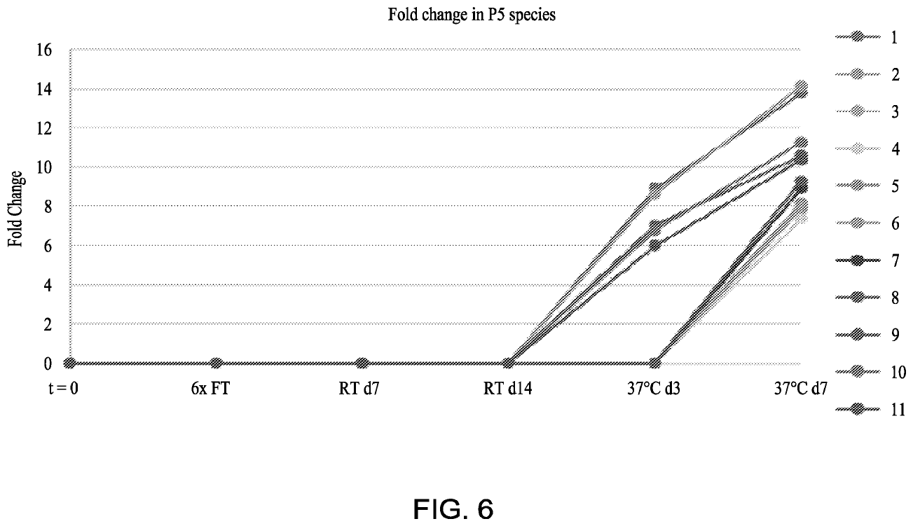

The samples were placed under each of the stress conditions described above and were quantitated for P1-P6 by RP-HPLC and compared to their time zero counterparts to determine the fold change (increase or decrease) of each peak. The fold-change in P1, P3, and P5 relative to t=0 for each sample and treatment are shown in FIGS. 4, 5 and 6, respectively. As shown in these figures, there was no significant change in P1, P3, or P5 in the 6×FT or 7 days at RT treatments, indicating stability in all the tested formulations through 6 freeze thaw cycles or 7 days at room temperature. After 14 days at room temperature P1 and P2 showed greater increases in samples 1, 2, 8, 9, and 10 than in samples 3-7, indicating the Buffer 2-based formulations were more stable at room temperature. Under higher thermal stress conditions (3 days at 37° C.), all formulations showed an increase in P1 and P2, with the greatest increases being in the Buffer 1-based formulations (1 and 2) followed by the Tris-based formulations (8-11). After 3 days at 37° C., P5 remained unchanged in the Buffer 2-based formulations (3-7), and increased in both Buffer 1-based formulations (1 and 2) and 3 of the 4 Buffer 3-based formulations (9-11). Under the highest thermal stress condition tested (7 days at 37° C.) all the formulations showed increases in P1, P3, and P5. The Buffer 1-based formulations showed the greatest increases in all three peaks, indicating these formulations had the lowest stability at 37° C. For P3 and P5 the lowest increases were observed in Buffer 2-based formulations, with higher increases seen in all Buffer 3-based formulations. For P1, the lowest increase was seen in formulations 6 and 4, the Buffer 2-based formulations. These results indicated that the rAAVs were more stable in the Buffer 2-based (3-7) formulations than in the Buffer 1 (1 and 2) or Buffer 3-based (8-11) formulations.

DLS analysis using the UNcle System was employed to determine particle size (or Z-average) and the polydispersity index (PDI) for each sample as a function of hold and stress conditions. In general, all samples displayed relatively uniform Z-average at 15° C. at t=0 and under all stress conditions (data not shown). While there was some variation in PDI at 15° C., all samples had a PDI <0.1 (data not shown). However, when tested at 95° C., differences between the samples were observed.

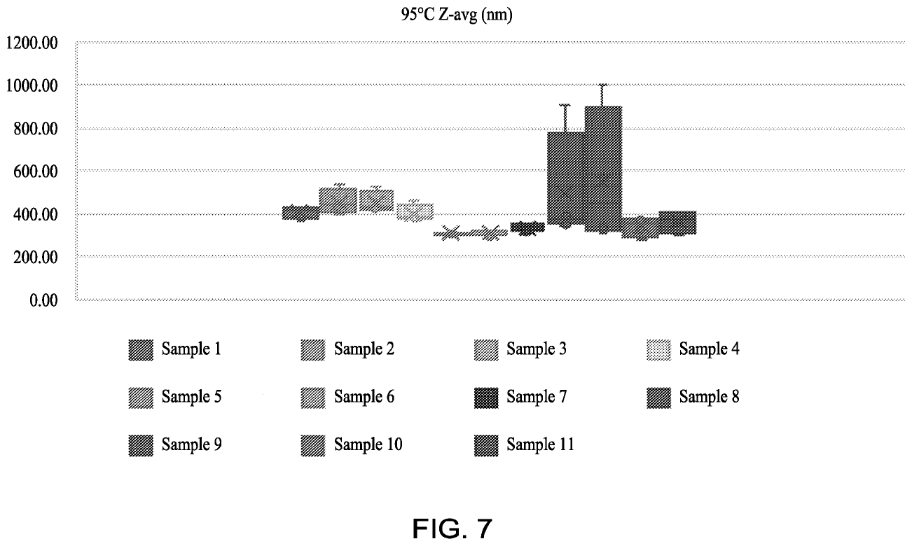

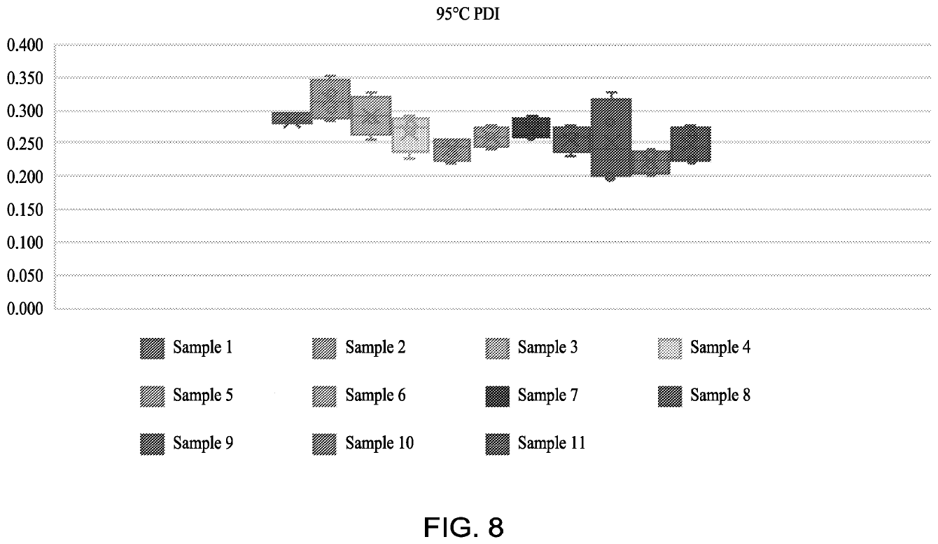

As shown in FIG. 7, the Z-averages of samples 5 and 6 were the lowest of all formulations tested with very narrow statistical variation, showing that these were the most stable formulations in this test. Samples 1~4 and 7-11 created larger aggregates or oligomers when stressed than samples 5 and 6, with Buffer 3-based samples 8 and 9 showing the least stability under stress conditions. As shown in FIG. 8, samples 5 and 6 had favorable statistical variation in PDI. Only sample 1 had a significantly narrower statistical variation in PDI. However, the significantly higher Z-average of sample 1 demonstrated that it was a less effective formulation for rAAV stability. Taken together, the Buffer 2-based samples with 0.005% P188 and MgCl₂ performed the best in this assay.

Based on the results of the assays above, Buffer 2-based formulations outperformed both Buffer 1- and Buffer 3-based formulations, with samples 5 and 6 displaying the highest level of thermal stress stability for rAAV. Formulation samples 5 and 6 demonstrated a high level of stability in the SEC analysis and the RP-HPLC analysis, and demonstrated a narrow statistical variation under stressed conditions in the PDI assay. Both of these sample formulations include 0.005% P188 and 0.8 mM MgCl₂.

### Example 4: Comparison Testing of Two rAAV Formulations

Two rAAV formulations were prepared for further testing. The formulation components for Formulation 1 and Formulation 2 are provided in Table 2. The two formulations both contained the same base buffer (1 mM sodium phosphate, 148 mM sodium chloride, 3 mM potassium chloride, 0.8 mM magnesium chloride, pH 7.30). The difference between Formulation 1 and Formulation 2 was the addition of 0.05% w/v Trehalose in Formulation 2. Both formulations (Formulation 1 and Formulation 2) were evaluated for vector genome titer by Digital Droplet Polymerase Chain Reaction (ddPCR), aggregation by Size Exclusion High Performance Liquid Chromatography (SE-HPLC), characterization by Reverse Phase High Performance Liquid Chromatography (RP-HPLC), in vitro potency, thermal transition analysis and dynamic light scattering (DLS) by UNcle, and subvisible particle (SVP) analysis. The conditions tested are summarized in Table 3.

TABLE 2
rAAV Formulation Buffers

| Formulation Buffer | Materials |
|---|---|
| Formulation 1 | 1 mM Phosphate [0.145 mM NaH₂PO₄ * |
| (Formulation | H₂0 and 0.854 mM Na₂HPO₄ * |
| Dilution Buffer) | 7H₂0], 148 mM NaCl, 3 mM KCl, and |
| | 0.005% Poloxamer 188; pH 7.3 |
| Formulation 2 | 1 mM Phosphate [0.145 mM NaH₂PO₄ * |
| | H₂0 and 0.854 mM Na₂HPO₄ * |
| | 7H₂0], 148 mM NaCl, 3 mM KCl, 0.005% |
| | Poloxamer 188, and 0.05% w/v Trehalose; pH 7.3 |

TABLE 3
Formulation Degradation Conditions

| Condition | Timepoint/Temperature |
|---|---|
| T = 0 | ≤−70° C. |

| | | |
|---|---|---|
| ≤−70° | C. | 1 month |

| | |
|---|---|
| 10 freeze/thaw cycles | ≤−70° C. and room temperature |

| | | |
|---|---|---|
| 4° | C. | 1 week and 1 month |
| 37° | C. | 2 weeks and 1 month |

| | |
|---|---|
| Room Temperature/ | 1 month |
| Ambient Stability | |

### Vector Genome Titer

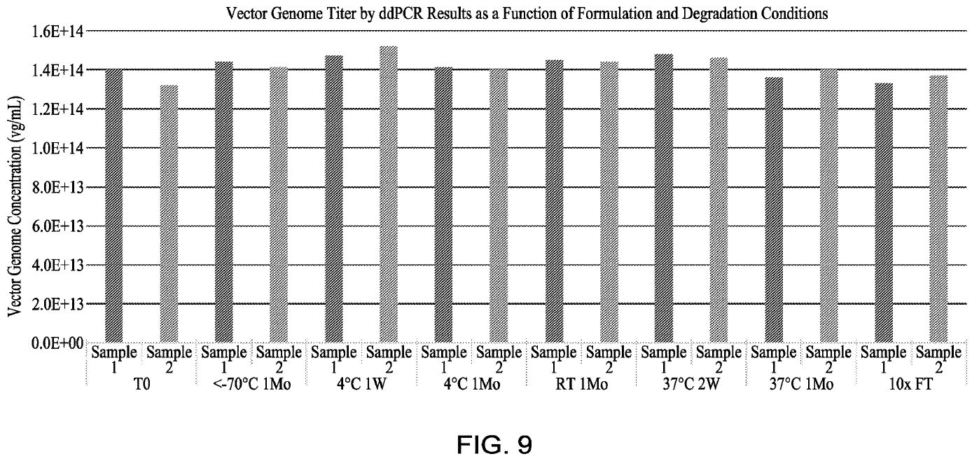

The vector genome titer was measured by UV absorbance and ddPCR. The initial vector concentration was determined by UV absorbance to achieve the final target concentration. After the initial concentration measurement, ddPCR was used to determine the vector genome titer for the formulation degradation conditions. ddPCR analysis produced genome titer data that was consistent across both formulations and within the variability of the assay. There were no discernable differences across the two formulations in their respective performance across the various stress and hold conditions. The vector genome titer results by ddPCR for the degradation conditions listed in Table are graphed in FIG. 9. The concentration ranged from 1.3E14-1.5E14 vg/mL.

### Aggregation

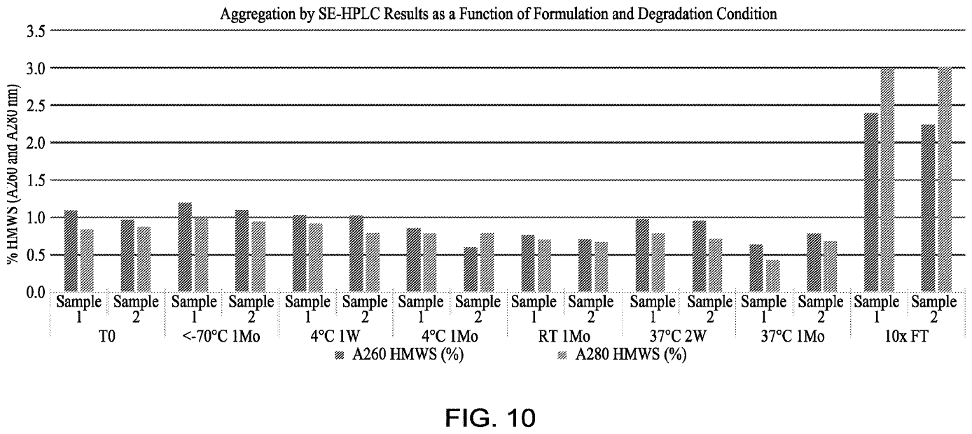

Aggregation of the rAAVs in the two formulations was measured by size exclusion high performance liquid chromatography (SE-HPLC) after a series of different sample treatments (storage at −70° C. for 1 month, storage at 4° C. for 1 week or 1 month, storage at room temperature for 1 month, storage at 37° C. for 2 weeks or 1 month, and 10 freeze thaw cycles. Low molecular weight species (LMWS) were not observed, and therefore the comparison was made between percent high molecular weight species (HMWS) at A260 and A280 nm. The % HMWS for the selected degradation conditions are graphed in FIG. 10. Formulations 1 and 2 showed little to no increase in HMWS during the different storage conditions, with both formulations peaking with HMWS of 3% or less after 10 freeze thaw cycles. These results indicate good stability of the rAAVs in both formulations.

### Characterization by Reversed-Phase HPLC (RP-HPLC)

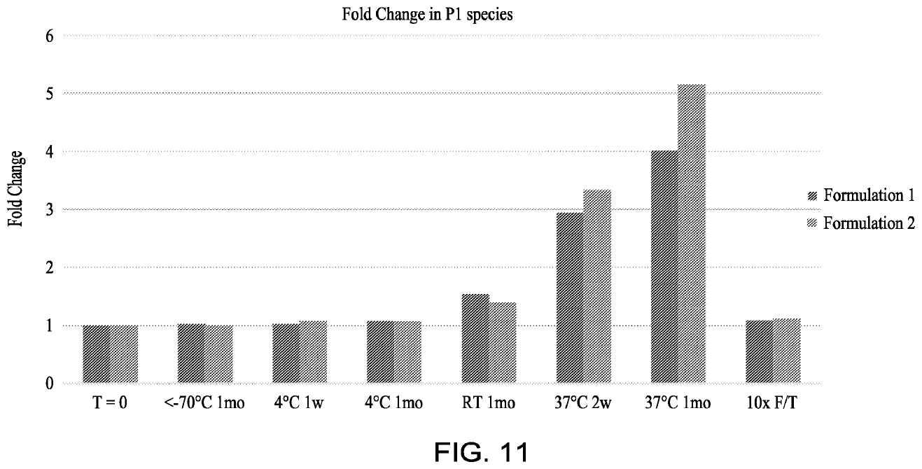

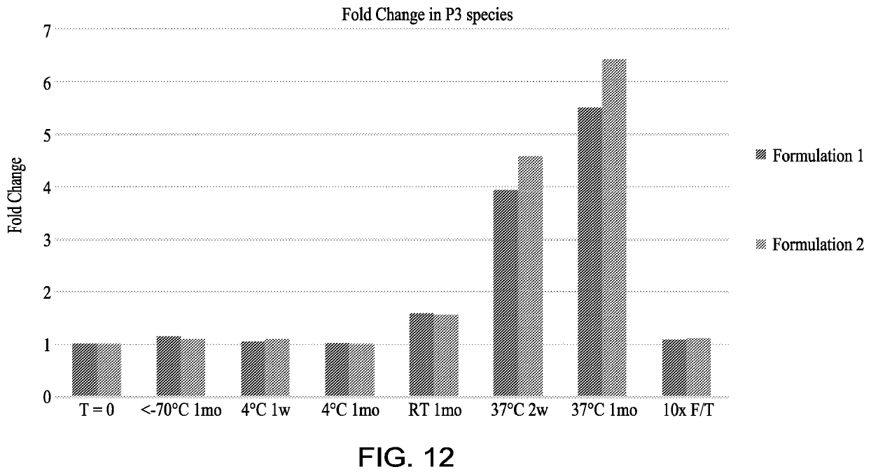

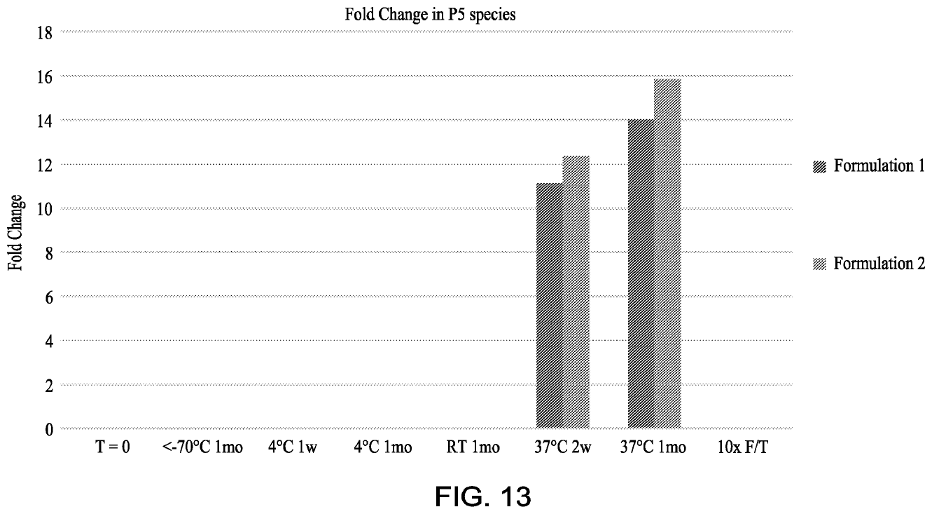

RP-HPLC was also used as a characterization method for the two tested formulations. Five peaks (P1-P4 and P6) were observed under non thermally stressed conditions and a sixth peak (P5) was apparent under thermal stress at 37° C. which increased in magnitude over time as shown in FIG. 13. Additionally, peaks one (FIG. 11) and three (FIG. 12) increased as a function of thermal stress, while peaks two, four, and six decreased. Peak five appears as a back shoulder peak off peak four. Peak four is represented by VP3 and is the largest quantity.

Overall, the thermal stressed samples exhibited the largest fold changes relative to T=0s in peak one, three, and five. Formulation 2 exhibited greater fold changes amongst the specified peaks in thermal stressed samples held at two weeks and one month.

### In Vitro Potency

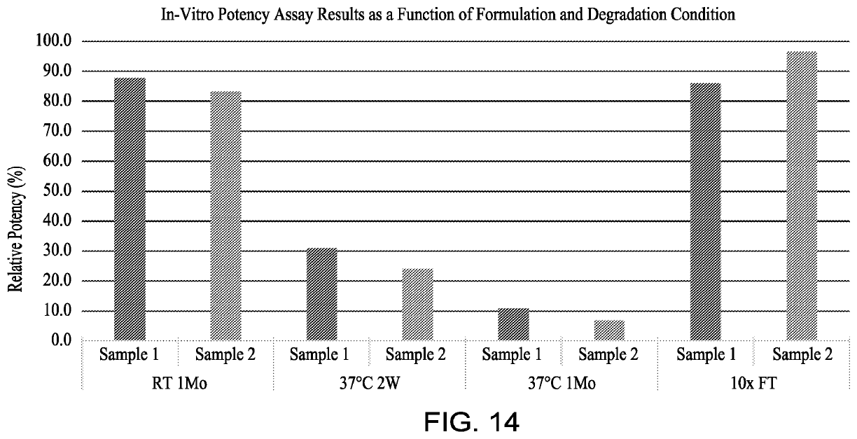

In vitro potency was analyzed for the room temperature/ambient stability, freeze-thaw, and thermal stressed conditions. Ten freeze thaw cycles had little effect on potency. Formulations subjected to thermal stress conditions demonstrated declines in potency. The potency results for the degradation conditions listed in Table 3 are graphed in FIG. 14. Both formulations showed similar levels of potency in the different conditions.

### Thermal Transition Analysis and Dynamic Light Scattering (DLS)

The UNcle system (Unchained Labs) is a system capable of multiple applications to characterize capsid particle stability as a function of formulation and degradative conditions. The DLS application was used to measure the particle size as a function of temperature, T_{agg} (aggregation temperature), and degree of polydispersity of capsid particles as a function of temperature at both 15° C. and 95° C. For monodispersed samples the Z-average (nm) is recommended by ISO standards from DLS analysis as the measurement is independent of distribution biases where results can vary dependent upon size classes in the fitting model. However, the Polydispersity Index (PDI) is also displayed as a measure of the degree of monodispersity of the sample whereas Z-average (nm) is the overall size distribution of the sample. Size and polydispersity before a thermal ramp are indicators of sample quality at the outset of an experiment and DLS after heating confirms the extent of aggregation observed between the two formulations at their respective degradation conditions.

In another application, analogous to a temperature-controlled differential scanning fluorimetry (DSF), thermal transition analysis was conducted to assess Tm (melting temperature) of the capsid particles measured using a DNA-fluorescent stain, Sybr Gold, as DNA leaks from the capsid during the thermal ramp. The assay is used to assess the multiple Tm stability points of the capsid particle and extrinsic fluorescence in the same assay. Extrinsic fluorescence measures the initial fluorescence intensity of residual non-encapsidated DNA present outside of the capsid as a byproduct of the rAAV manufacturing process, vector genome concentration, formulation, and degradation condition. The rAAVs of the formulations displayed two distinct biphasic Tm events. The first is designated as Tₒₙₛₑₜ or Tₘ₁ which represents the first instance where detectable genome is leaked from the capsid as a function of thermal instability during the thermal ramp to 95° C. The second event is designated as Tₘ₂ where the entirety of the capsid integrity is lost, and the full genome is released in solution.

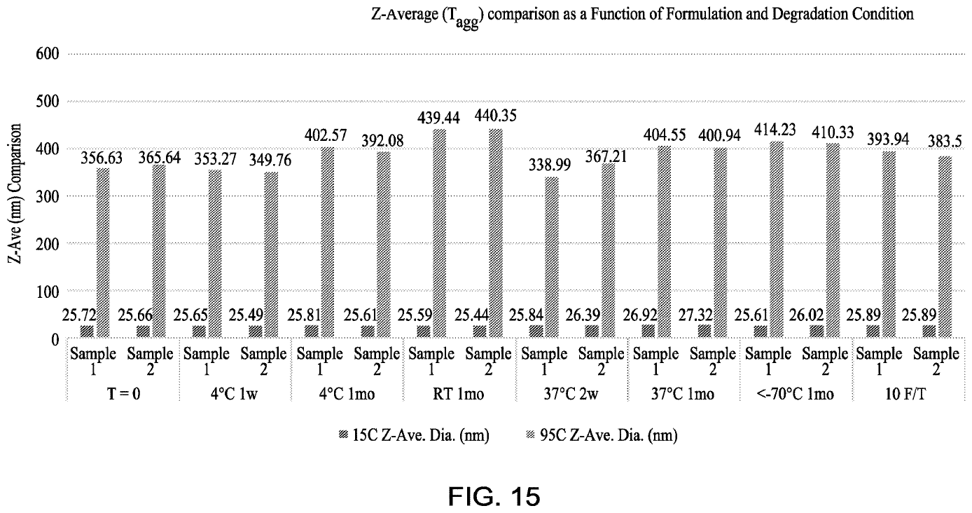

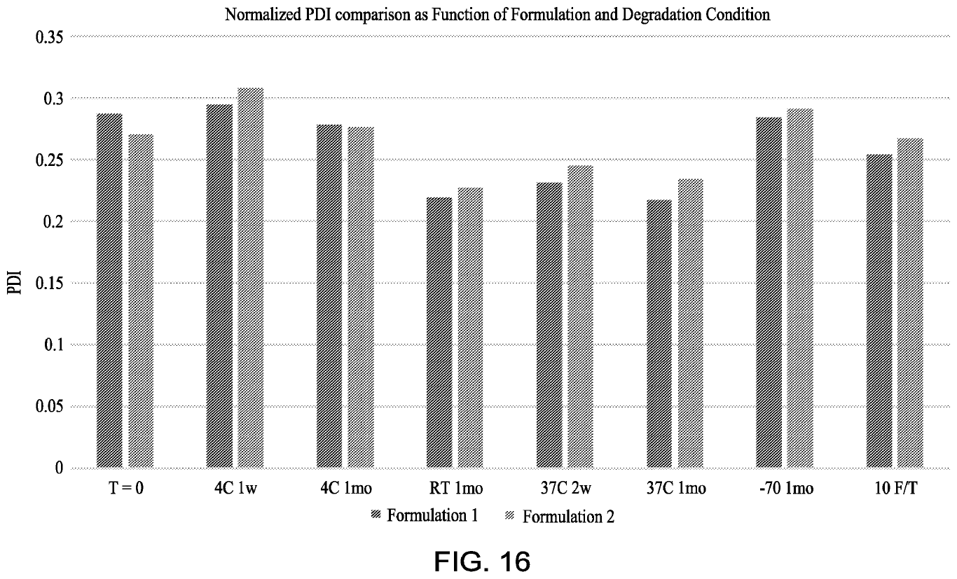

The Z-average (T_{agg}) and PDI results for the degradation conditions listed in Table 3 are graphed in FIG. 15 and FIG. 16. The T_{agg} based on the Z-average of Formulations 1 and 2 and their respective degradative conditions at both 15° C. and 95° C., as shown in FIG. 15, demonstrates similar aggregation between the formulations. The final measure of polydispersity at 95° C. was subtracted from the initial value at 15° C. to normalize the degree of polydispersity observed from the start and end of the thermal ramp to compare between the formulations and degradative conditions. As shown in FIG. 16, results were similar for the two formulations.

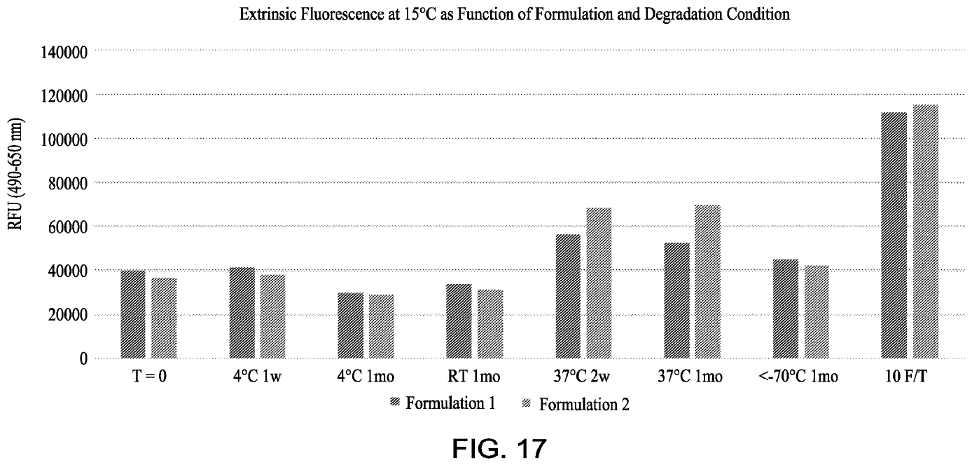

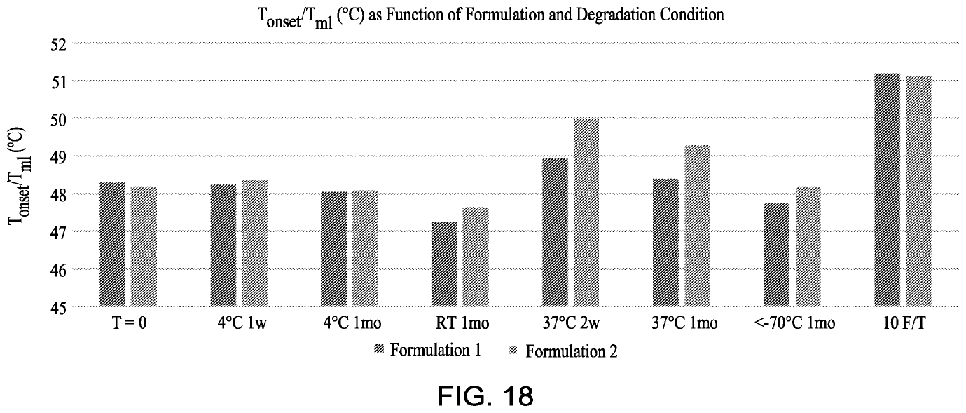

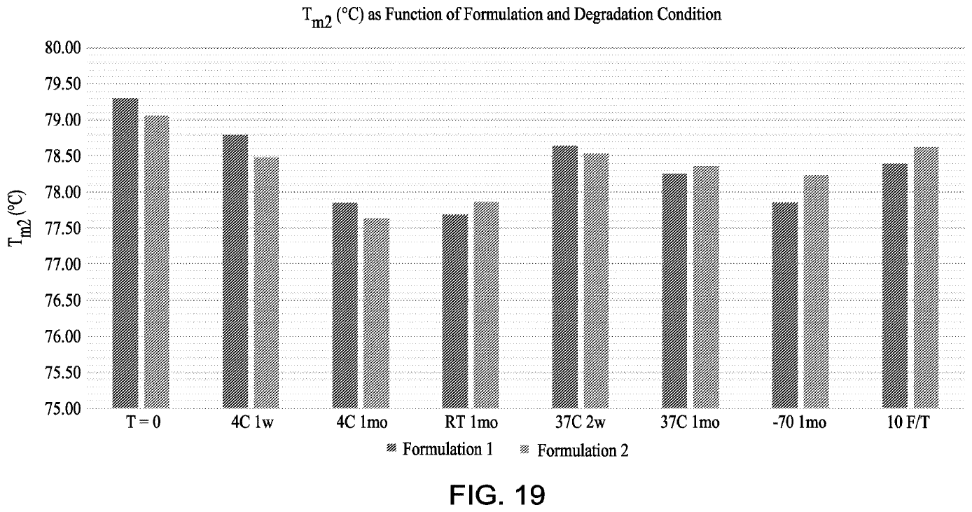

Assessment of thermal transition analysis with extrinsic fluorescence, Tₒₙₛₑₜ/Tₘ₁, and Tₘ₂ results are graphed in FIG. 17, FIG. 18, and FIG. 19, respectively, for the degradation conditions listed in Table 3. Extrinsic fluorescence was highest after ten freeze thaw cycles recapitulating prior observations of increased high molecular weight species by SE-HPLC. Additionally, between the two formulations there appears to be higher levels of fluorescence in Formulation 2 at 37° C., however the formulations appeared similar in the other degradation conditions. For Tₒₙₛₑₜ/Tₘ₁, and Tₘ₂, a difference of 2° C. is considered significant for Tm. Both formulations behaved similarly when assessing degradative conditions according to this metric.

### Subvisible Particle (SVP) Characterization

SVP was characterized using the Horizon system (Halo Labs, Burlingame, CA) which measures subvisible and visible particles at low volumes by using a backgrounded membrane imaging (BMI) technology. First an image is captured before samples are added to the membrane then aggregates that are too large to pass through the membrane and retained on the membrane for sizing and count distribution.

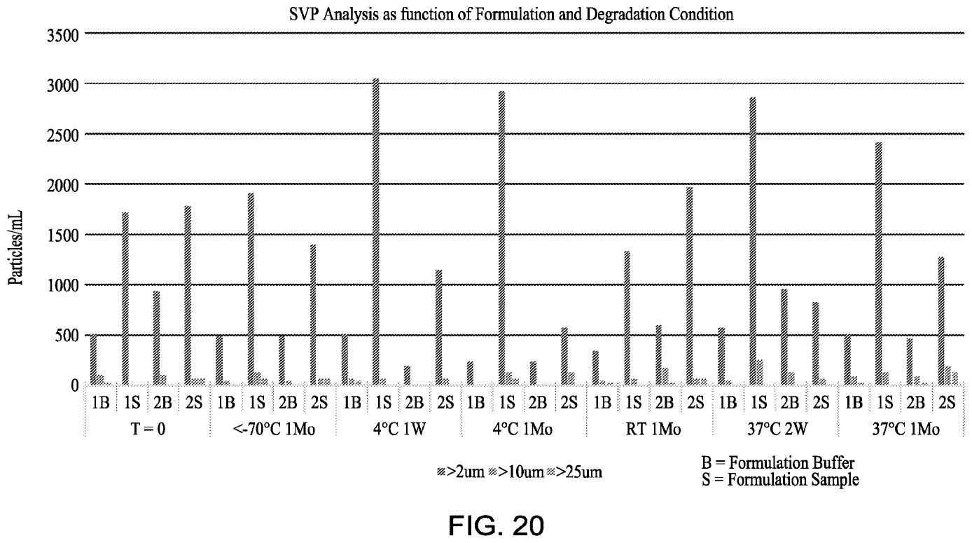

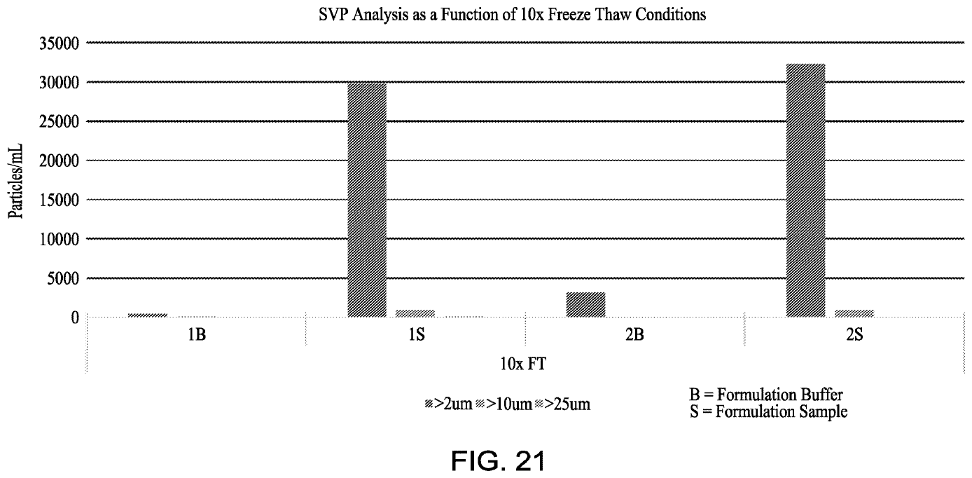

SVP analysis demonstrated that >2 μm particles were the most frequently observed sub-visible particle in all samples and conditions, followed by 10 μm and 25 μm particles as graphed in FIG. 20 and FIG. 21. The effective increase in SVP formation was more apparent under ten freeze thaw cycles where >2 and >10 μm particles increased relative to other conditions. Formulation 2 generally had lower SVP content in terms of >2 μm particles compared to Formulation 1 as a function of degradation specifically under <−70° C., 4° C., and 37° C. conditions. There is only a nominal increase in >25 μm particles as a function of ten freeze thaw conditions in Formulation 1 indicating trehalose in Formulation 2 may have a specific advantage as a cryoprotectant to reduce the larger particle formation.

### Formulation Degradation Discussion

Overall, there were very minor differences between the two formulations, demonstrating the specifically identified pH and ionic strength of the base formulation was adequate to prevent notable aggregation.

### Example 5: Comparison Testing of Further rAAV Formulations

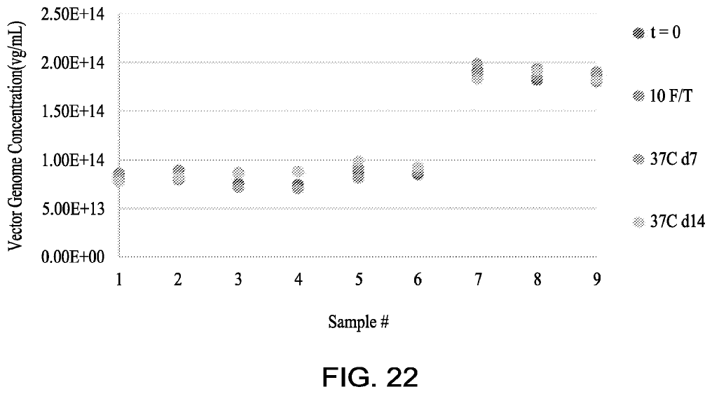

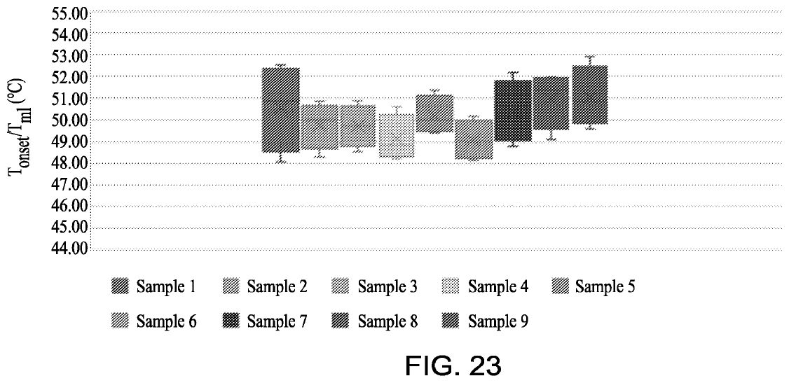

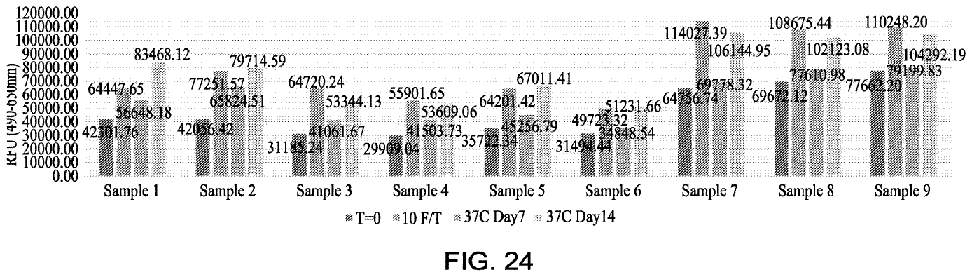

A further study was done with the formulations in Table 4. The different formulations in Table 4 were assessed as above. ddPCR analysis produced data for genome titer that was consistent across all candidate buffer conditions with no discernable loss of titer as a function of stress, as shown in FIG. 22. Thermal transition analysis by UNcle (Tₒₙₛₑₜ/Tₘ₁, FIG. 23, and extrinsic fluorescence, FIG. 24) of formulation samples showed similar results for samples 7 and 8 and did not indicate a benefit of the higher poloxamer 188 concentration with respect to genome titer. Similar results were also seen for samples 3 and 5, which did not indicate a benefit to higher poloxamer 188 concentrations with respect to genome titer.

TABLE 4
Formulation Samples

Sample Number

Formulation (at 8.00E+13 vg/mL)*

| | |
|---|---|
| 1 | 1x PBS + 0.001% P188 |
| 2 | Buffer 2 + 0.005% P188 |
| 3 | Buffer 2 + 0.005% P188 + 0.8 mM Mg |
| 4 | Buffer 2 + 0.005% P188 + |
| | 0.05% Trehalose + 0.8 mM Mg |
| 5 | Buffer 2 + 0.02% P188 + 0.8 mM Mg |
| 6 | Buffer 2 + 0.02% P188 + |
| | 0.05% Trehalose + 0.8 mM Mg |

Formulation (at 1.60E+14 vg/mL)

| | |
|---|---|
| 7 | Buffer 2 + 0.005% P188 + 0.8 mM Mg |
| 8 | Buffer 2 + 0.02% P188 + 0.8 mM Mg |
| 9 | Buffer 2 + 0.02% P188 + |
| | 0.05% Trehalose + 0.8 mM Mg |

*All Buffer 2 based formulations comprised 148 mM NaCl and 3 mM KCl in addition to components listed in the formulation column with the exception of 1x PBS (sample 1), which contained 10 mM sodium phosphate, 137 mM NaCl, and 3 mM KCl.

### Example 6: Poloxamer Loss in Processing

An experiment was conducted to assess poloxamer 188 loss under manufacturing conditions. rAAV was produced as in Example 1. Tangential Flow Filtration (TFF) was used to concentrate the rAAV material to a target concentration. Once the TFF filtrate had achieved the secondary concentration target, a 1% amount (v/v) of P188 Addition buffer was added to the final TFF concentrate material to target a concentration of 0.005% P188 in the material. The adjusted material was passed through a 0.2 μm low particle shedding filter. An aliquot of the final adjusted TFF concentrate was analyzed by ddPCR to determine the rAAV vector genome titer in the TFF concentrate.

Drug Substance (DS) was produced by diluting the adjusted TFF concentrate to the target concentration of 8.0E13 vg/mL with Formulation Dilution Buffer, and then filtering the DS through a 0.2 μm filter into a 125 mL polycarbonate final container. The ddPCR testing results from the TFF-B retentate sample were used to calculate the required volume of Formulation Dilution Buffer to reach the final DS target concentration. The required buffer was added to the adjusted TFF concentrate then mixed prior to filtration through a 0.2 μm filter into the final container. The DS was frozen.

The Drug Product (DP) manufacturing process initiated with a thaw of the DS containers in the 150 mL polycarbonate container. Once the DS had fully thawed, a titer sample was collected for testing by digital droplet polymerase chain reaction (ddPCR) testing. The thawed DS was maintained at 2-8° C. during the testing interval. The post-thaw DS titer value was utilized to determine a calculated volume of formulation dilution buffer to dilute the 8.0E13 DS to 2.0E13 vg/mL. Prior to performing the dilution, the thawed DS was removed from 2-8° C. conditions, returned to ambient conditions, and mixed. The full contents of the thawed DS were transferred to a 250 mL polycarbonate bottle and the calculated volume of formulation dilution buffer was added. The total diluted DS was mixed again prior to proceeding to sterile filtration. The filtration occurred from the DS bottle into a sterile single use filling bag.

The filtered bulk DP was filled using a semi-automated filler into sterile 2 mL Crystal Zenith® (CZ, cyclic olefin polymer) vials supplied by West Pharmaceutical Services (133MMX960), under aseptic conditions. The concentration of poloxamer 188 in the 2 ml vials was measured, and the results are shown in Table 5. Poloxamer was measured using HPLC-ELSD (High-Performance Liquid Chromatography Evaporative Light Scattering Detector).

The results described herein demonstrate that manufacturing processes associated with production of rAAV DP, such as the processes described herein, may result in loss of surfactant (here, poloxamer). As shown in Table 5, the final poloxamer concentrations after the processing steps ranged from 0.0034% to 0.0044%, representing poloxamer recovery rates of about 60% to about 88% (i.e., a loss of about 12% to about 40% of surfactant during processing).

TABLE 5
Poloxamer 188 losses in processing

| | | | Expected | Expected | Presumed |
|---|---|---|---|---|---|
| | Poloxamer | Poloxamer | Poloxamer | Poloxamer | Recovery of |
| Drug | 188 | 188 | 188 | 188 | P188 from |
| Substance | Concen- | Concen- | Concen- | Concen- | starting |
| Lot | tration | tration | tration | tration | concen- |
| Number | (μg/mL) | (%) | (μg/mL) | (%) | tration (%) |
| 1 | 39.6 | 0.00396 | 50.0 | 0.005 | 79.2 |
| 2 | 41.4 | 0.00414 | | | 82.8 |
| 3 | 37.4 | 0.00374 | | | 74.8 |
| 4 | 30.1 | 0.00301 | | | 60.2 |
| 5 | 31.8 | 0.00318 | | | 63.6 |
| 6 | 36.1 | 0.00361 | | | 72.2 |
| 7 | 34.1 | 0.00341 | | | 68.2 |
| 8 | 44.0 | 0.00440 | | | 88.0 |
| 9 | 40.0 | 0.00400 | | | 80.0 |
| 10 | 36.1 | 0.00361 | | | 72.2 |
| 11 | 40.7 | 0.00407 | | | 81.4 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context; the terms “a” (or “an”), “one or more,” and “at least one” can be used interchangeably herein. The term “or” should be understood to encompass items in the alternative or together, unless context unambiguously requires otherwise. The term “and/or” should be understood to encompass each item in a list (individually), any combination of items a list, and all items in a list together. The terms “comprising,” “having,” “including,” and “containing” are to be construed as open-ended terms (i.e., meaning “including, but not limited to,”) unless otherwise noted. The disclosure contemplates embodiments described as “comprising” a feature to include embodiments which “consist of” or “consist essentially of” the feature. The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 10%, up to 5%, or up to 1% of a given value.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. In any of the ranges described herein, the endpoints of the range are included in the range. However, the description also contemplates the same ranges in which the lower and/or the higher endpoint is excluded.

All method steps described herein can be performed in any suitable order unless otherwise indicated herein or otherwise clearly contradicted by context. The use of any and all examples, or exemplary language (e.g., “such as” and “optionally”) provided herein, is intended merely to better illuminate the disclosure and does not pose a limitation on the scope of the disclosure unless otherwise claimed. No language in the specification should be construed as indicating any non-claimed element as essential to the practice of the disclosure.

SEQUENCE KEY - 33236/55330

| SEQ | | |
|---|---|---|
| ID | Source | Sequence |
| 1 | SCNIB | 1 ccggtcccag agccgcagct gctgcgcccg cgcgctcccg gggacattct aaccgccgcc |
| | (NM_ | 61 aggtcccgcc gcctctcgcc ccgctattaa taccggcggc ccgggagggg ggcgcagcac |
| | 001037.4) | 121 gcgccgcgca gccatgggga ggctgctggc cttagtggtc ggcgcggcac tggtgtcctc |
| | | 181 agcctgcggg ggctgcgtgg aggtggactc ggagaccgag gccgtgtatg ggatgacctt |
| | | 241 caaaattctt tgcatctcct gcaagcgccg cagcgagacc aacgctgaga ccttcaccga |
| | | 301 gtggaccttc cgccagaagg gcactgagga gtttgtcaag atcctgcgct atgagaatga |
| | | 361 ggtgttgcag ctggaggagg atgagcgctt cgagggccgc gtggtgtgga atggcagccg |
| | | 421 gggcaccaaa gacctgcagg atctgtctat cttcatcacc aatgtcacct acaaccactc |
| | | 481 gggcgactac gagtgccacg tctaccgcct gctcttcttc gaaaactacg agcacaacac |
| | | 541 cagcgtcgtc aagaagatcc acattgaggt agtggacaaa gccaacagag acatggcatc |
| | | 601 catcgtgtct gagatcatga tgtatgtgct cattgtggtg ttgaccatat ggctcgtggc |
| | | 661 agagatgatt tactgctaca agaagatcgc tgccgccacg gagactgctg cacaggagaa |
| | | 721 tgcctcggaa tacctggcca tcacctctga aagcaaagag aactgcacgg gcgtccaggt |
| | | 781 ggccgaatag ccctggccct gggccccgcc tcaaggaaga gccagccgta atggggactc |
| | | 841 tccaggcacc gcctgccccc agcgtggggg tggccactcc tgggccccag aaagcctcag |
| | | 901 agtcctgccg acggagccac tggggtggga gggggcaggg ggcttggctc gcacccccac |
| | | 961 tttcgcctcc tccagctcct gccccgccgg ccgcgcaccg ccatgcatga tgggtaaagc |
| | | 1021 aatactgccg ctgcccccac cctgcttctg ctgcctgttt ggggaggggg gcggtgaggt |
| | | 1081 gggggcagcg gccccgcacc cctcctcctt gctgatttgc acacattggc cgcttcagac |
| | | 1141 acgcacttct ggggccagcc cctccccgcc tcctccctgc ctggcggcag gggtcgcgat |
| | | 1201 gatgggctgg agcagtttgg ggcagggggt tctgggaccc actccgactc cccctccccg |
| | | 1261 gcatcatttc ccctcccgct tcctccggct ggacctgggg tcccccctcc ctgtaatgca |
| | | 1321 ctcctgcccc ggcccaacct cgccctctct caccagcctt gaactgtggc cacctagaaa |
| | | 1381 ggggcccatt cagcctcgtc tctttacaga agtagttttg ttcatgaaat aaagactctt |
| | | 1441 ggacttgaaa aaaaaaaaaa aaa |
| 2 | SCNIA | 1 aatgtgcagg atgacaagat ggagcaaaca gtgcttgtac caccaggacc tgacagcttc |
| | (NM_ | 61 aacttcttca ccagagaatc tcttgcggct attgaaagac gcattgcaga agaaaaggca |
| | 001165963.1) | 121 aagaatccca aaccagacaa aaaagatgac gacgaaaatg gcccaaagcc aaatagtgac |
| | | 181 ttggaagctg gaaagaacct tccatttatt tatggagaca ttcctccaga gatggtgtca |
| | | 241 gagcccctgg aggacctgga cccctactat atcaataaga aaacttttat agtattgaat |
| | | 301 aaagggaagg ccatcttccg gttcagtgcc acctctgccc tgtacatttt aactcccttc |
| | | 361 aatcctctta ggaaaatagc tattaagatt ttggtacatt cattattcag catgctaatt |
| | | 421 atgtgcacta ttttgacaaa ctgtgtgttt atgacaatga gtaaccctcc tgattggaca |
| | | 481 aagaatgtag aatacacctt cacaggaata tatacttttg aatcacttat aaaaattatt |
| | | 541 gcaaggggat tctgtttaga agattttact ttccttcggg atccatggaa ctggctcgat |
| | | 601 ttcactgtca ttacatttgc gtacgtcaca gagtttgtgg acctgggcaa tgtctcggca |
| | | 661 ttgagaacat tcagagttct ccgagcattg aagacgattt cagtcattcc aggcctgaaa |
| | | 721 accattgtgg gagccctgat ccagtctgtg aagaagctct cagatgtaat gatcctgact |
| | | 781 gtgttctgtc tgagcgtatt tgctctaatt gggctgcagc tgttcatggg caacctgagg |
| | | 841 aataaatgta tacaatggcc tcccaccaat gcttccttgg aggaacatag tatagaaaag |
| | | 901 aatataactg tgaattataa tggtacactt ataaatgaaa ctgtctttga gtttgactgg |
| | | 961 aagtcatata ttcaagattc aagatatcat tatttcctgg agggtttttt agatgcacta |
| | | 1021 ctatgtggaa atagctctga tgcaggccaa tgtccagagg gatatatgtg tgtgaaagct |
| | | 1081 ggtagaaatc ccaattatgg ctacacaagc tttgatacct tcagttgggc ttttttgtcc |
| | | 1141 ttgtttcgac taatgactca ggacttctgg gaaaatcttt atcaactgac attacgtgct |
| | | 1201 gctgggaaaa cgtacatgat attttttgta ttggtcattt tcttgggctc attctaccta |
| | | 1261 ataaatttga tcctggctgt ggtggccatg gcctacgagg aacagaatca ggccaccttg |
| | | 1321 gaagaagcag aacagaaaga ggccgaattt cagcagatga ttgaacagct taaaaagcaa |
| | | 1381 caggaggcag ctcagcaggc agcaacggca actgcctcag aacattccag agagcccagt |
| | | 1441 gcagcaggca ggctctcaga cagctcatct gaagcctcta agttgagttc caagagtgct |
| | | 1501 aaggaaagaa gaaatcggag gaagaaaaga aaacagaaag agcagtctgg tggggaagag |
| | | 1561 aaagatgagg atgaattcca aaaatctgaa tctgaggaca gcatcaggag gaaaggtttt |
| | | 1621 cgcttctcca ttgaagggaa ccgattgaca tatgaaaaga ggtactcctc cccacaccag |
| | | 1681 tctttgttga gcatccgtgg ctccctattt tcaccaaggc gaaatagcag aacaagcctt |
| | | 1741 ttcagcttta gagggcgagc aaaggatgtg ggatctgaga acgacttcgc agatgatgag |
| | | 1801 cacagcacct ttgaggataa cgagagccgt agagattcct tgtttgtgcc ccgacgacac |
| | | 1861 ggagagagac gcaacagcaa cctgagtcag accagtaggt catcccggat gctggcagtg |
| | | 1921 tttccagcga atgggaagat gcacagcact gtggattgca atggtgtggt ttccttggtt |
| | | 1981 ggtggacctt cagttcctac atcgcctgtt ggacagcttc tgccagaggt gataatagat |
| | | 2041 aagccagcta ctgatgacaa tggaacaacc actgaaactg aaatgagaaa gagaaggtca |
| | | 2101 agttctttcc acgtttccat ggactttcta gaagatcctt cccaaaggca acgagcaatg |
| | | 2161 agtatagcca gcattctaac aaatacagta gaagaacttg aagaatccag gcagaaatgc |
| | | 2221 ccaccctgtt ggtataaatt ttccaacata ttcttaatct gggactgttc tccatattgg |
| | | 2281 ttaaaagtga aacatgttgt caacctggtt gtgatggacc catttgttga cctggccatc |
| | | 2341 accatctgta ttgtcttaaa tactcttttc atggccatgg agcactatcc aatgacggac |
| | | 2401 catttcaata atgtgcttac agtaggaaac ttggttttca ctgggatctt tacagcagaa |
| | | 2461 atgtttctga aaattattgc catggatcct tactattatt tccaagaagg ctggaatatc |
| | | 2521 tttgacggtt ttattgtgac gcttagcctg gtagaacttg gactcgccaa tgtggaagga |
| | | 2581 ttatctgttc tccgttcatt tcgattgctg cgagttttca agttggcaaa atcttggcca |
| | | 2641 acgttaaata tgctaataaa gatcatcggc aattccgtgg gggctctggg aaatttaacc |
| | | 2701 ctcgtcttgg ccatcatcgt cttcattttt gccgtggtcg gcatgcagct ctttggtaaa |
| | | 2761 agctacaaag attgtgtctg caagatcgcc agtgattgtc aactcccacg ctggcacatg |
| | | 2821 aatgacttct tccactcctt cctgattgtg ttccgcgtgc tgtgtgggga gtggatagag |
| | | 2881 accatgtggg actgtatgga ggttgctggt caagccatgt gccttactgt cttcatgatg |
| | | 2941 gtcatggtga ttggaaacct agtggtcctg aatctctttc tggccttgct tctgagctca |
| | | 3001 tttagtgcag acaaccttgc agccactgat gatgataatg aaatgaataa tctccaaatt |
| | | 3061 gctgtggata ggatgcacaa aggagtagct tatgtgaaaa gaaaaatata tgaatttatt |
| | | 3121 caacagtcct tcattaggaa acaaaagatt ttagatgaaa ttaaaccact tgatgatcta |
| | | 3181 aacaacaaga aagacagttg tatgtccaat catacagcag aaattgggaa agatcttgac |
| | | 3241 tatcttaaag atgtaaatgg aactacaagt ggtataggaa ctggcagcag tgttgaaaaa |
| | | 3301 tacattattg atgaaagtga ttacatgtca ttcataaaca accccagtct tactgtgact |
| | | 3361 gtaccaattg ctgtaggaga atctgacttt gaaaatttaa acacggaaga ctttagtagt |
| | | 3421 gaatcggatc tggaagaaag caaagagaaa ctgaatgaaa gcagtagctc atcagaaggt |
| | | 3481 agcactgtgg acatcggcgc acctgtagaa gaacagcccg tagtggaacc tgaagaaact |
| | | 3541 cttgaaccag aagcttgttt cactgaaggc tgtgtacaaa gattcaagtg ttgtcaaatc |
| | | 3601 aatgtggaag aaggcagagg aaaacaatgg tggaacctga gaaggacgtg tttccgaata |
| | | 3661 gttgaacata actggtttga gaccttcatt gttttcatga ttctccttag tagtggtgct |
| | | 3721 ctggcatttg aagatatata tattgatcag cgaaagacga ttaagacgat gttggaatat |
| | | 3781 gctgacaagg ttttcactta cattttcatt ctggaaatgc ttctaaaatg ggtggcatat |
| | | 3841 ggctatcaaa catatttcac caatgcctgg tgttggctgg acttcttaat tgttgatgtt |
| | | 3901 tcattggtca gtttaacagc aaatgccttg ggttactcag aacttggagc catcaaatct |
| | | 3961 ctcaggacac taagagctct gagacctcta agagccttat ctcgatttga agggatgagg |
| | | 4021 gtggttgtga atgccctttt aggagcaatt ccatccatca tgaatgtgct tctggtttgt |
| | | 4081 cttatattct ggctaatttt cagcatcatg ggcgtaaatt tgtttgctgg caaattctac |
| | | 4141 cactgtatta acaccacaac tggtgacagg tttgacatcg aagacgtgaa taatcatact |
| | | 4201 gattgcctaa aactaataga aagaaatgag actgctcgat ggaaaaatgt gaaagtaaac |
| | | 4261 tttgataatg taggatttgg gtatctctct ttgcttcaag ttgccacatt caaaggatgg |
| | | 4321 atggatataa tgtatgcagc agttgattcc agaaatgtgg aactccagcc taagtatgaa |
| | | 4381 gaaagtctgt acatgtatct ttactttgtt attttcatca tctttgggtc cttcttcacc |
| | | 4441 ttgaacctgt ttattggtgt catcatagat aatttcaacc agcagaaaaa gaagtttgga |
| | | 4501 ggtcaagaca tctttatgac agaagaacag aagaaatact ataatgcaat gaaaaaatta |
| | | 4561 ggatcgaaaa aaccgcaaaa gcctatacct cgaccaggaa acaaatttca aggaatggtc |
| | | 4621 tttgacttcg taaccagaca agtttttgac ataagcatca tgattctcat ctgtcttaac |
| | | 4681 atggtcacaa tgatggtgga aacagatgac cagagtgaat atgtgactac cattttgtca |
| | | 4741 cgcatcaatc tggtgttcat tgtgctattt actggagagt gtgtactgaa actcatctct |
| | | 4801 ctacgccatt attattttac cattggatgg aatatttttg attttgtggt tgtcattctc |
| | | 4861 tccattgtag gtatgtttct tgccgagctg atagaaaagt atttcgtgtc ccctaccctg |
| | | 4921 ttccgagtga tccgtcttgc taggattggc cgaatcctac gtctgatcaa aggagcaaag |
| | | 4981 gggatccgca cgctgctctt tgctttgatg atgtcccttc ctgcgttgtt taacatcggc |
| | | 5041 ctcctactct tcctagtcat gttcatctac gccatctttg ggatgtccaa ctttgcctat |
| | | 5101 gttaagaggg aagttgggat cgatgacatg ttcaactttg agacctttgg caacagcatg |
| | | 5161 atctgcctat tccaaattac aacctctgct ggctgggatg gattgctagc acccattctc |
| | | 5221 aacagtaagc cacccgactg tgaccctaat aaagttaacc ctggaagctc agttaaggga |
| | | 5281 gactgtggga acccatctgt tggaattttc ttttttgtca gttacatcat catatccttc |
| | | 5341 ctggttgtgg tgaacatgta catcgcggtc atcctggaga acttcagtgt tgctactgaa |
| | | 5401 gaaagtgcag agcctctgag tgaggatgac tttgagatgt tctatgaggt ttgggagaag |
| | | 5461 tttgatcccg atgcaactca gttcatggaa tttgaaaaat tatctcagtt tgcagctgcg |
| | | 5521 cttgaaccgc ctctcaatct gccacaacca aacaaactcc agctcattgc catggatttg |
| | | 5581 cccatggtga gtggtgaccg gatccactgt cttgatatct tatttgcttt tacaaagcgg |
| | | 5641 gttctaggag agagtggaga gatggatgct ctacgaatac agatggaaga gcgattcatg |
| | | 5701 gcttccaatc cttccaaggt ctcctatcag ccaatcacta ctactttaaa acgaaaacaa |
| | | 5761 gaggaagtat ctgctgtcat tattcagcgt gcttacagac gccacctttt aaagcgaact |
| | | 5821 gtaaaacaag cttcctttac gtacaataaa aacaaaatca aaggtggggc taatcttctt |
| | | 5881 ataaaagaag acatgataat tgacagaata aatgaaaact ctattacaga aaaaactgat |
| | | 5941 ctgaccatgt ccactgcagc ttgtccacct tcctatgacc gggtgacaaa gccaattgtg |
| | | 6001 gaaaaacatg agcaagaagg caaagatgaa aaagccaaag ggaaataaat gaaaataaat |
| | | 6061 aaaaataatt gggtgacaaa ttgtttacag cctgtgaagg tgatgtattt ttatcaacag |
| | | 6121 gactccttta ggaggtcaat gccaaactga ctgtttttac acaaatctcc ttaaggtcag |
| | | 6181 tgcctacaat aagacagtga ccccttgtca gcaaactgtg actctgtgta aaggggagat |
| | | 6241 gaccttgaca ggaggttact gttctcacta ccagctgaca ctgctgaaga taagatgcac |
| | | 6301 aatggctagt cagactgtag ggaccagttt caaggggtgc aaacctgtga ttttggggtt |
| | | 6361 gtttaacatg aaacacttta gtgtagtaat tgtatccact gtttgcattt caactgccac |
| | | 6421 atttgtcaca tttttatgga atctgttagt ggattcatct ttttgttaat ccatgtgttt |
| | | 6481 attatatgtg actatttttg taaacgaagt ttctgttgag aaataggcta aggacctcta |
| | | 6541 taacaggtat gccacctggg gggtatggca accacatggc cctcccagct acacaaagtc |
| | | 6601 gtggtttgca tgagggcatg ctgcacttag agatcatgca tgagaaaaag tcacaagaaa |
| | | 6661 aacaaattct taaatttcac catatttctg ggaggggtaa ttgggtgata agtggaggtg |
| | | 6721 ctttgttgat cttgttttgc gaaatccagc ccctagacca agtagattat ttgtgggtag |
| | | 6781 gccagtaaat cttagcaggt gcaaacttca ttcaaatgtt tggagtcata aatgttatgt |
| | | 6841 ttctttttgt tgtattaaaa aaaaaacctg aatagtgaat attgcccctc accctccacc |
| | | 6901 gccagaagac tgaattgacc aaaattactc tttataaatt tctgcttttt cctgcacttt |
| | | 6961 gtttagccat cttcggctct cagcaaggtt gacactgtat atgttaatga aatgctattt |
| | | 7021 attatgtaaa tagtcatttt accctgtggt gcacgtttga gcaaacaaat aatgacctaa |
| | | 7081 gcacagtatt tattgcatca aatatgtacc acaagaaatg tagagtgcaa gctttacaca |
| | | 7141 ggtaataaaa tgtattctgt accatttata gatagtttgg atgctatcaa tgcatgttta |
| | | 7201 tattaccatg ctgctgtatc tggtttctct cactgctcag aatctcattt atgagaaacc |
| | | 7261 atatgtcagt ggtaaagtca aggaaattgt tcaacagatc tcatttattt aagtcattaa |
| | | 7321 gcaatagttt gcagcacttt aacagctttt tggttatttt tacattttaa gtggataaca |
| | | 7381 tatggtatat agccagactg tacagacatg tttaaaaaaa cacactgctt aacctattaa |
| | | 7441 atatgtgttt agaattttat aagcaaatat aaatactgta aaaagtcact ttattttatt |
| | | 7501 tttcagcatt atgtacataa atatgaagag gaaattatct tcaggttgat atcacaatca |
| | | 7561 cttttcttac tttctgtcca tagtactttt tcatgaaaga aatttgctaa ataagacatg |
| | | 7621 aaaacaagac tgggtagttg tagatttctg ctttttaaat tacatttgct aattttagat |
| | | 7681 tatttcacaa ttttaaggag caaaataggt tcacgattca tatccaaatt atgctttgca |
| | | 7741 attggaaaag ggtttaaaat tttatttata tttctggtag tacctgcact aactgaattg |
| | | 7801 aaggtagtgc ttatgttatt tttgttcttt ttttctgact tcggtttatg ttttcatttc |
| | | 7861 tttggagtaa tgctgctcta gattgttcta aatagaatgt gggcttcata attttttttt |
| | | 7921 ccacaaaaac agagtagtca acttatatag tcaattacat caggacattt tgtgtttctt |
| | | 7981 acagaagcaa accataggct cctcttttcc ttaaaactac ttagataaac tgtattcgtg |
| | | 8041 aactgcatgc tggaaaatgc tactattatg ctaaataatg ctaaccaaca tttaaaatgt |
| | | 8101 gcaaaactaa taaagattac attttttatt tta |
| 3 | SCN2B | 1 ctctttttag cagcaacata caagccggcc atattagaga gatggaaata aagcttcctt |
| | (NM_ | 61 aatgttgtat atgtctttga agtacatccg tgcatttttt tttagcatcc aaccattcct |
| | 004588.4) | 121 cccttgtagt tctcgccccc tcaaatcacc ctctcccgta gcccacccga ctaacatctc |
| | | 181 agtctctgaa aatgcacaga gatgcctggc tacctcgccc tgccttcagc ctcacggggc |
| | | 241 tcagtctctt tttctctttg gtgccaccag gacggagcat ggaggtcaca gtacctgcca |
| | | 301 ccctcaacgt cctcaatggc tctgacgccc gcctgccctg caccttcaac tcctgctaca |
| | | 361 cagtgaacca caaacagttc tccctgaact ggacttacca ggagtgcaac aactgctctg |
| | | 421 aggagatgtt cctccagttc cgcatgaaga tcattaacct gaagctggag cggtttcaag |
| | | 481 accgcgtgga gttctcaggg aaccccagca agtacgatgt gtcggtgatg ctgagaaacg |
| | | 541 tgcagccgga ggatgagggg atttacaact gctacatcat gaacccccct gaccgccacc |
| | | 601 gtggccatgg caagatccat ctgcaggtcc tcatggaaga gccccctgag cgggactcca |
| | | 661 cggtggccgt gattgtgggt gcctccgtcg ggggcttcct ggctgtggtc atcttggtgc |
| | | 721 tgatggtggt caagtgtgtg aggagaaaaa aagagcagaa gctgagcaca gatgacctga |
| | | 781 agaccgagga ggagggcaag acggacggtg aaggcaaccc ggatgatggc gccaagtagt |
| | | 841 gggtggccgg ccctgcagcc tcccgtgtcc cgtctcctcc cctctccgcc ctgtacagtg |
| | | 901 accctgcctg ctcgctcttg gtgtgcttcc cgtgacctag gaccccaggg cccacctggg |
| | | 961 gcctcctgaa cccccgactt cgtatctccc accctgcacc aagagtgacc cactctcttc |
| | | 1021 catccgagaa acctgccatg ctctgggacg tgtgggccct ggggagagga gagaaagggc |
| | | 1081 tcccacctgc cagtccctgg ggggaggcag gaggcacatg tgagggtccc cagagagaag |
| | | 1141 ggagtgggtg ggcaggggta gaggaggggg ccgctgtcca cctgcccagt gcttggcctg |
| | | 1201 gcagtggctt cagagaggac ctggtgggga gggagggctt tcctgtgctg acagcgctcc |
| | | 1261 ctcaggaggg ccttggcctg gcacggctgt gctcctcccc tgctcccagc ccagagcagc |
| | | 1321 catcaggctg gaggtgacga tgagttcctg aaacttggag gggcatgtta aagggatgac |
| | | 1381 tgtgcattcc agggcactga cggaaagcca gggctgcagg caaagctgga catgtgccct |
| | | 1441 ggcccaggag gccatgttgg gccctcgttt ccattgctag tggcctcctt ggggctcccg |
| | | 1501 ttggctccta atcccttagg actgtggatg aggccagact ggaagagcag ctccaggtag |
| | | 1561 ggggccatgt ttcccagcgg ggacccacca acagaggcca gtttcaaagt cagctgaggg |
| | | 1621 gctgaggggt ggggctccat ggtgaatgca ggttgctgca ggctctgcct tctccatggg |
| | | 1681 gtaaccaccc tcgcctgggc aggggcagcc aaggctggga aatgaggagg ccatgcacag |
| | | 1741 ggtggggcag ctttctttgg ggcttcagtg agaactctcc cagttgccct tggtggggtt |
| | | 1801 tccacctggc ttttggctac agagagggaa gggaaagcct gaggccggca taaggggagg |
| | | 1861 ccttggaacc tgagctgcca atgccagccc tgtcccatct gcagccacac tactcgctcc |
| | | 1921 tctcccaaca actcccttcg tggggacaaa agtgacaatt gtaggccagg cacagtggct |
| | | 1981 cacgcctgta atcccagcac tttgggaggc caaggcgggt ggattacctc catctgttta |
| | | 2041 gtagaaatgg gcaaaacccc atctctacta aaaatacaag aattagctgg gcgtggtggc |
| | | 2101 gtgtgcctgt aatcccagct atttgggagg ctgaggcagg agaatcgctt gagcccggga |
| | | 2161 agcagaggtt gcagtgaact gagatagtga tagtgccact gcaattcagc ctgggtgaca |
| | | 2221 tagagagact ccatctcaaa aaaaaaaaaa aaaaaaaaaa aaggcaattg tgcggagcca |
| | | 2281 accatcactg ccattacact cacattcaat ggaaattgat taggcctgag agaaggagca |
| | | 2341 agaggcagaa acactgactc tgagaccatg accttggaag atcctcagag atcaactagc |
| | | 2401 caagtcactc acttaaaatg gggaaactga ggttccaact gacagaaact gagggtcctg |
| | | 2461 ccctcttctc tggctcctca gtctccagtt tgaagcaaca gagccatcat tttcccagtg |
| | | 2521 ggaggggcct ctgtgtcctg gtctatgatg gggcgagttg gaggggtggg cttctctagc |
| | | 2581 cctgccccct cccaggagcc cagcccctct ggccaggact gaggaggagt ggagggaagc |
| | | 2641 agggagggag ggagcaagtg tgaagggcac agctgctggc aaatgcccca ttccctgccc |
| | | 2701 cagcacttcc cagcctttcc aggagctctg ccccacccct actcaaggag tcaggtgccc |
| | | 2761 agggtaggga ataagtctag gaaagggggt ggagacagga ccgtgacagt ctcagggacc |
| | | 2821 aatccctgcc tctgcccatc agattgccaa tttggggaga cagcaatatc atctgggaga |
| | | 2881 ttcagcttaa tttgcttcaa ttcagcaacc atttattgag catctattat gtgccaggca |
| | | 2941 ctgtgccggg cactggggat atacacaaag ccataagcac tggggactgc caggttggca |
| | | 3001 ctgtagggac caagattaac tttttggtgc atctaattcc acctctgcaa gacactgctt |
| | | 3061 ccaaggctac agttggggaa gatgaagaga ttctagaatc tgtgccctgg gaagggagaa |
| | | 3121 acagcaggga agaggggcaa tacaacaggg tcaagggtgc agaggccagg cagagtgggt |
| | | 3181 gcagagtggg ggatgggcaa ccagcctggt aacacctggg tgaggtgagc gagtcaagcc |
| | | 3241 catctggaaa tgctaaactc atcagctcag tacctaactg ccaatcaaca cactgccaat |
| | | 3301 gcgggtgggc taagccgcca ggatgcctgc agcagatgga cgctgcccat ccttgagcca |
| | | 3361 gccagctggg ggcagagagg tgggtggcgg gcacctcatg ccagctgaga ccaagagtgg |
| | | 3421 ttgtatacat ttctgcagct tgcagatttc atttttttgg tcacccttta agtgtccttc |
| | | 3481 tctcccagga tgagtgagga gctgtgctga tgttgggaac ttatcttttt ccaggatcca |
| | | 3541 taacatttag gggaattatt tgcgggcttt cctagtgtat tctaagaagt tctaactctc |
| | | 3601 catcaagaag cccaagattc ctggagtctt tttttcctct gccctgatca actggggccc |
| | | 3661 aggaaaagca atgtctcttc acgggcctca gggccccagc agatggggct gggtcaggaa |
| | | 3721 ccatctctgt cccaggtgct cagccctcat atgcattttc ttctggagcc tcttagaatc |
| | | 3781 tcctccagag gactcataga gctgcctccc ccaggatggt gaagttggga gcaaaatggg |
| | | 3841 gcttctgtaa gtagaggtgg aaaaccagag agatgtttcc acagcagcca tttccctacc |
| | | 3901 ctgtttccta acttatgctg acaagttctt cctcttccta ccgtggggtt cgagccccac |
| | | 3961 ttctggttgc atgttcccac caccacgctc tgctggtttt agtcggctga ctttctcaac |
| | | 4021 tgccccagcc tctcctgctc tctcagaacc tccctctgcc cccacccccc atctgtctct |
| | | 4081 tcctcctaat taagggccac tgcaaagaga gacgggctgg ctaacccatg ggatttagct |
| | | 4141 tataagaaca aagcacaatt attcaggact gaactgcttt gaggtgcctt ggaactggct |
| | | 4201 ttggaaatag ctatctgccc cctagctatg gactcttcac agttatttcc agtttataaa |
| | | 4261 aaggaaaagc tgatcggatc catgtttctc tctgcgagtc tccttgggga ggttgggctg |
| | | 4321 gtaaatattt gcaaggtcag gttaagcaga cagacaattg aaaatactgg gcaggtccaa |
| | | 4381 aaggatagtt ccaggccata ggaatggctt ggtgcagtta acgggtgatg aagcaagaaa |
| | | 4441 tcttccttcc tacttatgtc ttaaagatgg acagcctgtg tctcttgctg tgcctcttgc |
| | | 4501 tggcgtcttc ctttattgaa gcttgctgat gcactgtaca tagtctctct ctctaggtat |
| | | 4561 agatatatta tatatacaaa tatcaaacat ctcaccacac ccaccccaaa agttacactg |
| | | 4621 tgtgtgtgtg gggaggtgta ataacttctc agtgttttga gttgattcac caaaggcaaa |
| | | 4681 ttttggaatg ttcttgcgct ttgtgcactt ggaggggaaa acccaactac atatgcactt |
| | | 4741 tcttggttgt gtgtgctata gagaggttgg aaggggtggt tggaagggga gagaaactga |
| | | 4801 gatggccctc tgtgactcga ctgctgggat gtatctgctt ttgggagcag actgagtttc |
| | | 4861 ttttgcaatt tgtcttattg tttgggtcta ctggcaacaa taaactatct cccctgaaaa |
| | | 4921 tc |
| 4 | KCNC1 | 1 ggcggccgct cccatgggtg tcgctgggcc gcgccatgcc taagggggcg ccgcgatggg |
| | (NM_ | 61 ccaaggggac gagagcgagc gcatcgtgat caacgtgggc ggcacgcgcc accagacgta |
| | 001112741.1) | 121 ccgctcgacc ctgcgcacgc tgcccggcac gcggctcgcc tggctggcgg agcccgacgc |
| | | 181 ccacagccac ttcgactatg acccgcgtgc tgacgagttc ttcttcgacc gccaccccgg |
| | | 241 cgtcttcgcg cacatcctga actactaccg cacgggcaag ctgcactgcc cagccgacgt |
| | | 301 gtgcgggccg ctctacgagg aggagctggc cttctggggc atcgacgaga ccgacgtgga |
| | | 361 gccctgctgc tggatgacgt accgccagca ccgcgacgcc gaggaggctc tggacagctt |
| | | 421 cggcggcgct cctctggaca acagcgccga cgacgcggac gccgacggcc ctggcgactc |
| | | 481 gggcgacggc gaggacgagc tggagatgac caagcgcctg gcgctcagtg actccccgga |
| | | 541 tggccggcct ggcggctttt ggcgccgctg gcagccgcgc atctgggcgc tcttcgagga |
| | | 601 cccgtactcg tcccgctacg cgcggtatgt ggccttcgct tccctcttct tcatcctggt |
| | | 661 ctccatcacc accttctgcc tggagaccca cgagcgcttc aaccccatcg tgaacaagac |
| | | 721 ggagatcgag aacgttcgca atggcacgca agtgcgctac taccgggagg ccgagacgga |
| | | 781 ggccttcctt acctacatcg agggcgtctg tgtggtctgg ttcaccttcg agttcctcat |
| | | 841 gcgtgtcatc ttctgcccca acaaggtaga gttcatcaag aactcgctca acatcattga |
| | | 901 ctttgtggcc atcctgccct tctacctgga ggtggggctg agcggcctgt cctccaaggc |
| | | 961 agccaaggac gtgctgggct tcctgcgcgt cgtccgcttc gtgcgcatct tgcgcatctt |
| | | 1021 taagctgacc cgccactttg tgggcctgcg ggtcctgggc cacacgctcc gagccagcac |
| | | 1081 caacgagttc ctgctgctca tcatcttcct ggccttgggc gtgctgatct tcgccaccat |
| | | 1141 gatctactac gccgagagga taggggcaca gcccaatgac cccagcgcca gtgagcacac |
| | | 1201 gcactttaag aacatcccca tcggcttctg gtgggccgtg gtcaccatga cgaccctggg |
| | | 1261 ctatggagac atgtacccgc agacgtggtc cggcatgctg gtgggggctc tgtgtgcgct |
| | | 1321 ggcgggcgtg ctcaccatcg ccatgcccgt gcccgtcatc gtgaacaatt tcgggatgta |
| | | 1381 ttactcctta gccatggcta agcagaaact accaaagaaa aaaaagaagc atattccgcg |
| | | 1441 gccaccgcag ctgggatctc ccaattattg taaatctgtc gtaaactctc cacaccacag |
| | | 1501 tactcagagt gacacatgtc cgctggccca ggaagaaatt ttagaaatta acagagcaga |
| | | 1561 ttccaaactg aatggggagg tggcgaaggc cgcgctggcg aacgaagact gcccccacat |
| | | 1621 agaccaggcc ctcactcccg atgagggcct gccctttacg cgctcgggca cccgcgagag |
| | | 1681 atacggaccc tgcttcctct tatcaaccgg ggagtacgcg tgcccacctg gtggaggaat |
| | | 1741 gagaaaggat ctttgcaaag aaagccctgt cattgctaag tatatgccga cagaggctgt |
| | | 1801 gagagtgact tgaccaggcg gcttggccga ggacactggt ggctattaag catctgggtg |
| | | 1861 gacctgcagc ccctcctcac cctcggacag agtaaattca cgccatgcag gtttgccgga |
| | | 1921 cgagtccgag tggcccaggc attgtactag gacggacgta gctttttcta cggccaaatg |
| | | 1981 gtaactgacc gtagaggatt tcttttcctt cttttcattt tttaaaattt tattttattt |
| | | 2041 ggggaggggg ggtggagggg ctccttagca tgacttgcat gaagttaaac agaaaaccca |
| | | 2101 gcaaaccaaa cccaccaacc ccctgcaact gtatgattac cctgaacaac aataatgaaa |
| | | 2161 agaaaaacat caaagccctc tattttcttt caaagctctt gactttcaca cacgttgttg |
| | | 2221 gagccaaact gtaacggcgt tcagtagaaa cctgtacatt tctgggggtg aggggcgagg |
| | | 2281 ggaggaggga cagagatggg gaatggattg cttccttttt gtacacaaga tcaagaggaa |
| | | 2341 agcttccaaa aagggcagtc ggtcagtcat cggtcatatt gacctcacct tcatagttca |
| | | 2401 tctctcacgt ggaaactgag aactttgctc caaatagaat tgtggaaact cgcacggctg |
| | | 2461 ccttcttccc cgtttggcat gcttgtgacc cagcggatgt ctccccaaga cccctgacag |
| | | 2521 cagctagtct aggttgattc tctcatctta tcggtgtgta gatccagtgt gaccaacttt |
| | | 2581 cataacaaat tgttcatagt aaataatcac caccgtatgg attttccaag tgttccatta |
| | | 2641 aaaccaggat gtagagcacc aaaagatcag gaccacggga ggagcagcct cctccacctt |
| | | 2701 tactaaggca caacctggca ttgtatgcaa tttagtactt cagagtaaaa acctgcatgc |
| | | 2761 ccaatgttat gcaaagcgat tctagcatga aataaatttt gtatcatggt ttctgtatag |
| | | 2821 tctaaagcag atttgttttc ctgaagcagt cggaggtttg cagagacaca cttctgcatc |
| | | 2881 tcgaataaat atagtatttt cccaacagaa acagaggaca gtagcttggc tttggtctga |
| | | 2941 ttctaaaatt agtggggtgg gggggaagga tgatattttt gaaaaacaca tgcttgagtt |
| | | 3001 gaaaaaaatg caacatctca agctttcact gcagctcaca acatttcctg gaaagagagc |
| | | 3061 aatgatgctt tagctcaaca acctcccctc cctgggccct ggcccccaat aataaaaaac |
| | | 3121 tgacttttga gatg |
| 5 | KCNC3 | 1 accggtcccg cctcccttga cgccccgccc cgcccctcct ccctgctctc ttagaaagag |
| | (NM_004977.2) | 61 ccaatcgctc cgtctcattg gcggccccga ctcgtccagc tgggtcgcgt tagagtgccc |
| | | 121 gccccactcc tcccagccac agccccgccc cacctccccc aggccaatca gtttggttcc |
| | | 181 tctcccctaa gccacgcccc cgctacctcg ctcctcctc cacccaatcc cgtcggtctc |
| | | 241 tccccccgtg tccccgcccc tgcgtccccg cccctcccgc cccgcccccc gtccaatgct |
| | | 301 gagctcagtc tgcgtctcgt ccttccgcgg gcgccagggg gccagcaagc agcagccggc |
| | | 361 gccaccgccg cagccgcccg agtccccgcc gccgccaccg ctgccgccgc agcagcagca |
| | | 421 gcctgcgcag cccggccccg ccgcgtcccc ggcgggcccc ccggcacccc gcgggcccgg |
| | | 481 ggaccggcgc gccgagccat gccccgggct gccggcggcg gccatggggc ggcacggcgg |
| | | 541 cggcggtggc gacagcggca agatcgtgat caacgtgggc ggcgtgcgcc atgagacgta |
| | | 601 ccgctcgacg ctgcgcaccc tgccggggac gcggctggcc ggcctgacgg agcccgaggc |
| | | 661 ggcggcacgc ttcgactacg acccgggcgc cgacgagttc ttctttgacc ggcacccggg |
| | | 721 agtcttcgcg tacgtgctca actactaccg caccggcaag ctgcactgcc cagccgacgt |
| | | 781 gtgcgggccc ctgtttgagg aggagctcgg cttctggggc atcgacgaga ccgacgtgga |
| | | 841 ggcctgctgc tggatgacct accggcagca tcgcgacgct gaggaggcgc tcgactcctt |
| | | 901 cgaggcgccc gaccccgcgg gcgccgccaa cgccgccaac gccgcaggcg cccacgacgg |
| | | 961 aggcctggac gacgaggcgg gcgcgggcgg cggcggcctg gacggagcgg gcggcgagct |
| | | 1021 caagcgcctc tgcttccagg acgcgggcgg cggcgccggg gggccgccag ggggcgcggg |
| | | 1081 cggcgcgggc ggcacatggt ggcgccgctg gcagccccgc gtgtgggcgc tcttcgagga |
| | | 1141 cccctactcg tcgcgggctg ccaggtatgt ggccttcgcc tccctcttct tcatcctcat |
| | | 1201 ctccatcacc accttctgcc tggaaaccca tgagggcttc atccatatta gcaacaagac |
| | | 1261 ggtgacccag gcctccccga tccccggggc acctccggag aacatcacca acgtggaggt |
| | | 1321 ggagacggag cccttcctga cctacgtgga gggggtgtgc gtggtctggt tcaccttcga |
| | | 1381 gttcctcatg cgcatcacct tctgcccaga caaggtggag tttcttaaaa gcagcctcaa |
| | | 1441 catcatcgac tgtgtggcca tcctgccctt ctatctcgag gtgggcctct cgggcctcag |
| | | 1501 ctccaaggcc gccaaagacg tgctgggctt cctgcgggtg gtccgcttcg tccgcatcct |
| | | 1561 gcgcatcttc aagctgaccc ggcacttcgt ggggctgcgc gtgctgggac acacgctccg |
| | | 1621 cgccagcacc aacgagttcc tgctgctcat catcttcctg gccctggggg tgctcatctt |
| | | 1681 cgccaccatg atttactacg ctgagcgcat tggcgccgac cccgatgaca tcctgggctc |
| | | 1741 caaccacacc tacttcaaga acatccccat tggcttctgg tgggctgtgg tcaccatgac |
| | | 1801 gaccctgggc tatggagaca tgtaccccaa gacgtggtcg gggatgctgg tcggggcgct |
| | | 1861 gtgtgccctg gcgggggtgc tgaccatcgc catgcctgtg cccgtcattg tcaacaactt |
| | | 1921 tggcatgtac tattcgctgg ccatggccaa gcagaagctg cccaagaaga agaacaaaca |
| | | 1981 catcccccgg cccccgcaac cgggctcgcc caactactgc aagcctgacc cacccccgcc |
| | | 2041 acccccgccc cacccgcacc acggcagcgg gggcatcagc ccgccgccac ccatcacccc |
| | | 2101 accctccatg ggggtgactg tggccggggc ctacccagcg gggccccaca cgcaccccgg |
| | | 2161 gctgctcagg gggggagcgg gtgggctggg gatcatgggg ctgcctcctc tgccagcccc |
| | | 2221 cggcgagcct tgcccgttgg ctcaggagga ggtgattgag atcaaccggg cagatcctcg |
| | | 2281 ccccaatggg gatccggcag cagctgcgct tgcccacgag gactgcccag ccattgacca |
| | | 2341 gcctgccatg tccccggaag acaagagccc catcacgcct ggaagccgtg gccgctatag |
| | | 2401 ccgggaccga gcctgcttcc tcctcaccga ctatgcccct tcccctgatg gctccatccg |
| | | 2461 aaaagccact ggtgctcccc cactgccccc ccaagactgg cgtaagccag gccccccaag |
| | | 2521 cttcttgccc gacctcaacg ccaacgccgc ggcctggata tccccctagt ggacgaaccc |
| | | 2581 cctccccccg ggctcttgtc accgcctgag acctcgcgag actttcggtc ccccccgccc |
| | | 2641 cttcccccca ggttagcaat tgggaatggc tgggaggggg tgtccccaag acactgggct |
| | | 2701 tcaaatctcc ccccaagccc tccctictac gatcaagaca ccttttgtcc agacagctcc |
| | | 2761 cttagcgttg cctggaaaag ccacagagct ccctgcgggg gtgcagaacc ctgcaggcag |
| | | 2821 ccccaggctt tgcagaaacc acacacactg ttgctggatc ttgagtccca ttagctgtga |
| | | 2881 gatgcctgtg agagccttcc cgcacccgtc agaagctcag agccttcttg gaccctttaa |
| | | 2941 agatgtccct ctccccagcc ctccgcctca ccgcccccca cccccgaccc cgtccttaga |
| | | 3001 atcttctgga ggagcctccc tcccccccca ccccccccca ctgctcctgg agttctcatc |
| | | 3061 ggattcccca agggcactgc cagcttcgct ccactcagcc cccttgcaga ccccaccccc |
| | | 3121 tgcctgctct ctttccctac aactaggtca gcccccagcc ccgctacggc ggccgc |
| 6 | SCN1A sequence; | DFEMFYEVWEKFDPDATQFMEFEKLSQFAAALEPPLNLPQPNKLQLIAMDLPMVSGDRIH |
| | SEQ ID NO: 39 | CLDILFAFTKRVLGESGEMDALRIQMEERFMASNPSKVSYQPITTTLKRKQEEVSAVIIQ |
| | from | RAYRRHLLKRTVKQASFTYNKNKIKGGANLLIKEDMIIDRINENSITEKTDLTMSTAACP |
| | WO 2018187363 | PSYDRVTKPIVEKHEQEGKDEKAKGK |
| 7 | SEQ ID NO: 77 | MEQTVLVPPGPDSFNFFTRESLAAIERRIAEEKAKNPKPDKKDDDENGPKPNSDLEAGKN |
| | of WO | LPFIYGDIPPEMVSEPLEDLDPYYINKKTFIVLNKGKAIFRFSATSALYILTPFNPLRKI |
| | 2020243651 | AIKILVHSLFSMLIMCTILTNCVFMTMSNPPDWTKNVEYTFTGIYTFESLIKIIARGFCL |
| | | EDFTFLRDPWNWLDFTVITFAYVTEFVDLGNVSALRTFRVLRALKTISVIPGLKTIVGAL |
| | | IQSVKKLSDVMILTVFCLSVFALIGLQLFMGNLRNKCIQWPPTNASLEEHSIEKNITVNY |
| | | NGTLINETVFEFDWKSYIQDSRYHYFLEGFLDALLCGNSSDAGQCPEGYMCVKAGRNPNY |
| | | GYTSFDTFSWAFLSLFRLMTQDFWENLYQLTLRAAGKTYMIFFVLVIFLGSFYLINLILA |
| | | VVAMAYEEQNQATLEEAEQKEAEFQQMIEQLKKQQEAAQQAATATASEHSREPSAAGRLS |
| | | DSSSEASKLSSKSAKERRNRRKKRKQKEQSGGEEKDEDEFQKSESEDSIRRKGFRFSIEG |
| | | NRLTYEKRYSSPHQSLLSIRGSLFSPRRNSRTSLFSFRGRAKDVGSENDFADDEHSTFED |
| | | NESRRDSLFVPRRHGERRNSNLSQTSRSSRMLAVFPANGKMHSTVDCNGVVSLVGGPSVP |
| | | TSPVGQLLPEVIIDKPATDDNGTTTETEMRKRRSSSFHVSMDFLEDPSQRQRAMSIASIL |
| | | TNTVEELEESRQKCPPCWYKFSNIFLIWDCSPYWLKVKHVVNLVVMDPFVDLAITICIVL |
| | | NTLFMAMEHYPMTDHFNNVLTVGNLVFTGIFTAEMFLKIIAMDPYYYFQEGWNIFDGFIV |
| | | TLSLVELGLANVEGLSVLRSFRLLRVFKLAKSWPTLNMLIKIIGNSVGALGNLTLVLAII |
| | | VFIFAVVGMQLFGKSYKDCVCKIASDCQLPRWHMNDFFHSFLIVFRVLCGEWIETMWDCM |
| | | EVAGQAMCLTVFMMVMVIGNLVVLNLFLALLLSSFSADNLAATDDDNEMNNLQIAVDRMH |
| | | KGVAYVKRKIYEFIQQSFIRKQKILDEIKPLDDLNNKKDSCMSNHTAEIGKDLDYLKDVN |
| | | GTTSGIGTGSSVEKYIIDESDYMSFINNPSLTVTVPIAVGESDFENLNTEDFSSESDLEE |
| | | SKEKLNESSSSSEGSTVDIGAPVEEQPVVEPEETLEPEACFTEGCVQRFKCCQINVEEGR |
| | | GKQWWNLRRTCFRIVEHNWFETFIVFMILLSSGAFEDIYIDQRKTIKTMLEYADKVFTYI |
| | | FIMLLKWVAYGYQTYFTNAWCWLDFLIVDVSLVSLTANALGYSELGAIKSLRTLRALRPL |
| | | RALSRFEGMRVVVNALLGAIPSIMNVLLVCLIFWLIFSIMGVNLFAGKFYHCINTTTGDR |
| | | FDIEDVNNHTDCLKLIERNETARWKNVKVNFDNVGFGYLSLLQVATFKGWMDIMYAAVDS |
| | | RNVELQPKYEESLYMYLYFVIFIIFGSFFTLNLFIGVIIDNFNQQKKKFGGQDIFMTEEQ |
| | | KKYYNAMKKLGSKKPQKPIPRPGNKFQGMVFDFVTRQVFDISIMILICLNMVTMMVETDD |
| | | QSEYVTTILSRINLVFIVLFTGECVLKLISLRHYYFTIGWNIFDFVVVILSIVGMFLAEL |
| | | IEKYFVSPTLFRVIRLARIGRILRLIKGAKGIRTLLFALMMSLPALFNIGLLLFLVMFIY |
| | | AIFGMSNFAYVKREVGIDDMFNFETFGNSMICLFQITTSAGWDGLLAPILNSKPPDCDPN |
| | | KVNPGSSVKGDCGNPSVGIFFFVSYIIISFLVVVNMYIAVINFSVATEESAEPLSEDLEP |
| | | GEKPYKCPECGKSFSRSDNLVRHQRTHTGEKPYKCPECGKSFSREDNLHTHQRTHTGEKP |
| | | YKCPECGKSFSRSDELVRHQRTHTGEKPYKCPECGKSFSQSGNLTEHQRTHTGEKPYKCP |
| | | ECGKSFSTSGHLVRHQRTHTGEKPYKCPECGKSFSQNSTLTEHQRTHTGKKTS |
| 8 | SEQ ID NO: 78 of WO | LEPGEKPYKCPECGKSFSTKNSLTEHQRTHTGEKPYKCPECGKSFSRADNLTEHQRTHTG |
| | 2020243651 | EKPYKCPECGKSFSQLAHLRAHQRTHTGEKPYKCPECGKSFSTKNSLTEHQRTHTGEKPY |
| | | KCPECGKSFSQAGHLASHQRTHTGEKPYKCPECGKSFSTHLDLIRHQRTHTGKKTS |
| 9 | SEQ ID NO: 79 of WO | LEPGEKPYKCPECGKSFSQAGHLASHQRTHTGEKPYKCPECGKSFSREDNLHTHQRTHTG |
| | 2020243651 | EKPYKCPECGKSFSTSGNLTEHQRTHTGEKPYKCPECGKSFSTHLDLIRHQRTHTGEKPY |
| | | KCPECGKSFSQKSSLIAHQRTHTGEKPYKCPECGKSFSQAGHLASHQRTHTGKKTS |
| 10 | SEQ ID NO: 80 of WO | LEPGEKPYKCPECGKSFSTTGNLTVHQRTHTGEKPYKCPECGKSFSTSGELVRHQRTHTG |
| | 2020243651 | EKPYKCPECGKSFSREDNLHTHQRTHTGEKPYKCPECGKSFSTSGNLTEHQRTHTGEKPY |
| | | KCPECGKSFSQSSSLVRHQRTHTGEKPYKCPECGKSFSQRANLRAHQRTHTGKKTS |
| 11 | SEQ ID NO: 81 of WO | LEPGEKPYKCPECGKSFSSRRTCRAHQRTHTGEKPYKCPECGKSFSTTGALTEHQRTHTG |
| | 2020243651 | EKPYKCPECGKSFSRSDELVRHQRTHTGEKPYKCPECGKSFSRNDALTEHQRTHTGEKPY |
| | | KCPECGKSFSQSGDLRRHQRTHTGEKPYKCPECGKSFSTSHSLTEHQRTHTGKKTS |
| 12 | SEQ ID NO: 82 of WO | LEPGEKPYKCPECGKSFSRKDNLKNHQRTHTGEKPYKCPECGKSFSDPGALVRHQRTHTG |
| | 2020243651 | EKPYKCPECGKSFSREDNLHTHQRTHTGEKPYKCPECGKSFSDPGALVRHQRTHTGEKPY |
| | | KCPECGKSFSTSGELVRHQRTHTGEKPYKCPECGKSFSRKDNLKNHQRTHTGKKTS |
| 13 | SEQ ID NO: 83 of WO | LEPGEKPYKCPECGKSFSSKKALTEHQRTHTGEKPYKCPECGKSFSSPADLTRHQRTHTG |
| | 2020243651 | EKPYKCPECGKSFSRSDNLVRHQRTHTGEKPYKCPECGKSFSREDNLHTHQRTHTGEKPY |
| | | KCPECGKSFSRSDELVRHQRTHTGEKPYKCPECGKSFSQSGNLTEHQRTHTGEKPYKCPE |
| | | CGKSFSTSGHLVRHQRTHTGEKPYKCPECGKSFSQNSTLTEHQRTHTGKKTS |
| 14 | SEQ ID NO: 84 of WO | LEPGEKPYKCPECGKSFSSPADLTRHQRTHTGEKPYKCPECGKSFSRSDNLVRHQRTHTG |
| | 2020243651 | EKPYKCPECGKSFSREDNLHTHQRTHTGEKPYKCPECGKSFSRSDELVRHQRTHTGEKPY |
| | | KCPECGKSFSQSGNLTEHQRTHTGEKPYKCPECGKSFSTSGHLVRHQRTHTGKKTS |
| 15 | SEQ ID NO: 85 of WO | LEPGEKPYKCPECGKSFSSKKALTEHQRTHTGEKPYKCPECGKSFSSPADLTRHQRTHTG |
| | 2020243651 | EKPYKCPECGKSFSRSDNLVRHQRTHTGEKPYKCPECGKSFSREDNLHTHQRTHTGEKPY |
| | | KCPECGKSFSRSDELVRHQRTHTGEKPYKCPECGKSFSQSGNLTEHQRTHTGKKTS |
| 16 | SEQ ID NO: 86 of WO | LEPGEKPYKCPECGKSFSDCRDLARHQRTHTGEKPYKCPECGKSFSRNDALTEHQRTHTG |
| | 2020243651 | EKPYKCPECGKSFSRNDALTEHQRTHTGEKPYKCPECGKSFSSPADLTRHQRTHTGEKPY |
| | | KCPECGKSFSDPGNLVRHQRTHTGEKPYKCPECGKSFSQRAHLERHQRTHTGEKPYKCPE |
| | | CGKSFSQSSSLVRHQRTHTGEKPYKCPECGKSFSHRTTLTNHQRTHTGKKTS |
| 17 | SEQ ID NO: 87 of WO | LEPGEKPYKCPECGKSFSRNDALTEHQRTHTGEKPYKCPECGKSFSSPADLTRHQRTHTG |
| | 2020243651 | EKPYKCPECGKSFSDPGNLVRHQRTHTGEKPYKCPECGKSFSQRAHLERHQRTHTGEKPY |
| | | KCPECGKSFSQSSSLVRHQRTHTGEKPYKCPECGKSFSHRTTLTNHQRTHTGKKTS |
| 18 | SEQ ID NO: 88 of WO | LEPGEKPYKCPECGKSFSRNDALTEHQRTHTGEKPYKCPECGKSFSDPGHLVRHQRTHTG |
| | 2020243651 | EKPYKCPECGKSFSTSGELVRHQRTHTGEKPYKCPECGKSFSTHLDLIRHQRTHTGEKPY |
| | | KCPECGKSFSSKKALTEHQRTHTGEKPYKCPECGKSFSQLAHLRAHQRTHTGEKPYKCPE |
| | | CGKSFSRSDHLTNHQRTHTGKKTS |
| 19 | SEQ ID NO: 89 of WO | LEPGEKPYKCPECGKSFSRSDNLVRHQRTHTGEKPYKCPECGKSFSHRTTLTNHQRTHTG |
| | 2020243651 | EKPYKCPECGKSFSREDNLHTHQRTHTGEKPYKCPECGKSFSTSHSLTEHQRTHTGEKPY |
| | | KCPECGKSFSQSSSLVRHQRTHTGEKPYKCPECGKSFSREDNLHTHQRTHTGKKTS |
| 20 | SEQ ID NO: 90 of WO | LEPGEKPYKCPECGKSFSDPGALVRHQRTHTGEKPYKCPECGKSFSRSDNLVRHQRTHTG |
| | 2020243651 | EKPYKCPECGKSFSQSGDLRRHQRTHTGEKPYKCPECGKSFSTHLDLIRHQRTHTGEKPY |
| | | KCPECGKSFSTSGNLVRHQRTHTGEKPYKCPECGKSFSRSDNLVRHQRTHTGKKTS |
| 21 | SEQ ID NO: 91 of WO | LEPGEKPYKCPECGKSFSRRDELNVHQRTHTGEKPYKCPECGKSFSRSDHLTNHQRTHTG |
| | 2020243651 | EKPYKCPECGKSFSRSDDLVRHQRTHTGEKPYKCPECGKSFSRSDNLVRHQRTHTGEKPY |
| | | KCPECGKSFSHRTTLTNHQRTHTGEKPYKCPECGKSFSREDNLHTHQRTHTGEKPYKCPE |
| | | CGKSFSTSHSLTEHQRTHTGEKPYKCPECGKSFSQSSSLVRHQRTHTGEKPYKCPECGKS |
| | | FSREDNLHTHQRTHTGKKTS |
| 22 | SEQ ID NO: 92 of WO | RPYACPVESCDRRFSRSDNLVRHIRIHTGQKPFQCRICMRNFSREDNLHTHIRTHTGEKP |
| | 2020243651 | FACDICGRKFARSDELVRHTKIHLRQKDRPYACPVESCDRRFSQSGNLTEHIRIHTGQKP |
| | | FQCRICMRNFSTSGHLVRHIRTHTGEKPFACDICGRKFAQNSTLTEHTKIHLRQKDK |
| 23 | SEQ ID NO: 93 of WO | RPYACPVESCDRRFSRSDNLVRHIRIHTGQKPFQCRICMRNFSHRTTLTNHIRTHTGEKP |
| | 2020243651 | FACDICGRKFAREDNLHTHTKIHLRQKDRPYACPVESCDRRFSTSHSLTEHIRIHTGQKP |
| | | FQCRICMRNFSQSSSLVRHIRTHTGEKPFACDICGRKFAREDNLHTHTKIHLRQKDK |
| 24 | SEQ ID NO: 94 of WO | RPYACPVESCDRRFSRRDELNVHIRIHTGQKPFQCRICMRNFSRSDHLTNHIRTHTGEKP |
| | 2020243651 | FACDICGRKFARSDDLVRHTKIHLRQKDRPYACPVESCDRRFSRSDNLVRHIRIHTGQKP |
| | | FQCRICMRNFSHRTTLTNHIRTHTGEKPFACDICGRKFAREDNLHTHTKIHLRQKDRPYA |
| | | CPVESCDRRFSTSHSLTEHIRIHTGQKPFQCRICMRNFSQSSSLVRHIRTHTGEKPFACD |
| | | ICGRKFAREDNLHTHTKIHLRQKD |
| 25 | SEQ ID NO: 95 of WO | RPYACPVESCDRRFSDPGALVRHIRIHTGQKPFQCRICMRNFSRSDNLVRHIRTHTGEKP |
| | 2020243651 | FACDICGRKFAQSGDLRRHTKIHLRQKDRPYACPVESCDRRFSTHLDLIRHIRIHTGQKP |
| | | FQCRICMRNFSTSGNLVRHIRTHTGEKPFACDICGRKFARSDNLVRHTKIHLRQKDRPYA |
| | | CPVESCDRRFSQSGHLTEHIRIHTGQKPFQCRICMRNFSERSHLREHIRTHTGEKPFACD |
| | | ICGRKFAQAGHLASHTKIHLRQKD |
| 26 | SEQ ID NO: 96 of WO | RPHACPAEGCDRRFSRSDNLVRHLRIHTGHKPFQCRICMRSFSREDNLHTHIRTHTGEKP |
| | 2020243651 | FACEFCGRKFARSDELVRHAKIHLKQKEHACPAEGCDRRFSQSGNLTEHLRIHTGHKPFQ |
| | | CRICMRSFSTSGHLVRHIRTHTGEKPFACEFCGRKFAQNSTLTEHAKIHLKQKEK |
| 27 | SEQ ID NO: 97 of WO | RPHACPAEGCDRRFSRSDNLVRHLRIHTGHKPFQCRICMRSFSHRTTLTNHIRTHTGEKP |
| | 2020243651 | FACEFCGRKFAREDNLHTHAKIHLKQKEHACPAEGCDRRFSTSHSLTEHLRIHTGHKPFQ |
| | | CRICMRSFSQSSSLVRHIRTHTGEKPFACEFCGRKFAREDNLHTHAKIHLKQKEK |
| 28 | SEQ ID NO: 98 of WO | RPHACPAEGCDRRFSRRDELNVHLRIHTGHKPFQCRICMRSFSRSDHLTNHIRTHTGEKP |
| | 2020243651 | FACEFCGRKFARSDDLVRHAKIHLKQKEHACPAEGCDRRFSRSDNLVRHLRIHTGHKPFQ |
| | | CRICMRSFSHRTTLTNHIRTHTGEKPFACEFCGRKFAREDNLHTHAKIHLKQKEHACPAE |
| | | GCDRRFSTSHSLTEHLRIHTGHKPFQCRICMRSFSQSSSLVRHIRTHTGEKPFACEFCGR |
| | | KFAREDNLHTHAKIHLKQKEK |
| 29 | VP64 (SEQ ID NO: 95 | DALDDFDLDMLGSDALDDFDLDMLGSDALDDFDLDMLGSDALDDFDLDML |
| | from WO | |
| | 2019/109051) | |
| 30 | VPR (SEQ ID NO: | DALDDFDLDMLGSDALDDFDLDMLGSDALDDFDLDMLGSDALDDFDLDMLINSRSSGSPK |
| | 114 from WO | KKRKVGSQYLPDTDDRHRIEEKRKRTYETFKSIMKKSPFSGPTDPRPPPRRIAVPSRSSA |
| | 2019/109051) | SVPKPAPQPYPFTSSLSTINYDEFPTMVFPSGQISQASALAPAPPQVLPQAPAPAPAPAM |
| | | VSALAQAPAPVPVLAPGPPQAVAPPAPKPTQAGEGTLSEALLQLQFDDEDLGALLGNSTD |
| | | PAVFTDLASVDNSEFQQLLNQGIPVAPHTTEPMLMEYPEAITRLVTGAQRPPDPAPAPLG |
| | | APGLPNGLLSGDEDFSSIADMDFSALLGSGSGSRDSREGMFLPKPEAGSAISDVFEGREV |
| | | CQPKRIRPFHPPGSPWANRPLPASLAPTPTGPVHEPVGSLTPAPVPQPLDPAPAVTPEAS |
| | | HLLEDPDEETSQAVKALREMADTVIPQKEEAAICGQMDLSHPPPRGHLDELTTTLESMTE |
| | | DLNLDSPLTPELNEILDTFLNDECLLHAMHISTGLSIFDTSLF |
| 31 | CBP/p300-interacting | MSGLEMADHMMAMNHGRFPDGTNGLHHHPAHRMGMGQFPSPHHHQQQQPQHAFNALMGEH |
| | transactivator 2 | IHYGAGNMNATSGVRHAMGPGTVNGGHPPSALAPAARFNNSQFMGPPVASQGGSLPASMQ |
| | (CITED2) (SEQ ID | LQKLNNQYFNHHPYPHNHYMPDLHPAAGHQMNGTNQHFRDCNPKHSGGSSTPGGSGGSST |
| | NO: 96 from WO | PGGSGSSSGGGAGSSNSGGGSGSGNMPASVAHVPAAMLPPNVIDTDFIDEEVLMSLVIEM |
| | 2019/109051) | GLDRIKELPELWLGQNEFDFMTDFVCKQQPSRVSC |
| 32 | CBP/p300-interacting | SGLEMADHMMAMNHGRFPDGTNGLHHHPAHRMGMGQFPSPHHHQQQQPQHAFNALMGEHI |
| | transactivator 2 | HYGAGNMNATSGVRHAMGPGTVNGGHPPSALAPAARFNNSQFMGPPVASQGGSLPASMQL |
| | (CITED2) (SEQ ID | QKLNNQYFNHHPYPHNHYMPDLHPAAGHQMNGTNQHFRDCNPKHSGGSSTPGGSGGSSTP |
| | NO: 98 from WO | GGSGSSSGGGAGSSNSGGGSGSGNMPASVAHVPAAMLPPNVIDTDFIDEEVLMSLVIEMG |
| | 2019/109051) | LDRIKELPELWLGQNEFDFMTDFVCKQQPSRVSC |
| 33 | CBP/p300-interacting | MADHLMLAEGYRLVQRPPSAAAAHGPHALRTLPPYAGPGLDSGLRPRGAPLGPPPPRQPG |
| | transactivator 4 | ALAYGAFGPPSSFQPFPAVPPPAAGIAHLQPVATPYPGRAAAPPNAPGGPPGPQPAPSAA |
| | (CITED4) (SEQ ID | APPPPAHALGGMDAELIDEEALTSLELELGLHRVRELPELFLGQSEFDCFSDLGSAPPAG |
| | NO: 97 from WO | SVSC |
| | 2019/109051) | |
| 34 | CBP/p300-interacting | AADHLMLAEGYRLVQRPPSAAAAHGPHALRTLPPYAGPGLDSGLRPRGAPLGPPPPRQPG |
| | transactivator 4 | ALAYGAFGPPSSFQPFPAVPPPAAGIAHLQPVATPYPGRAAAPPNAPGGPPGPQPAPSAA |
| | (CITED4) (SEQ ID | APPPPAHALGGMDAELIDEEALTSLELELGLHRVRELPELFLGQSEFDCFSDLGSAPPAG |
| | NO: 100 from WO | SVSC |
| | 2019/109051) | |
| 35 | EGR1 (SEQ ID NO: 1 | MAAAKAEMQLMSPLQISDPFGSFPHSPTMDNYPKLEEMMLLSNGAPQFLGAAGAPEGSGS |
| | from WO | NSSSSSSGGGGGGGGGSNSSSSSSTFNPQADTGEQPYEHLTAESFPDISLNNEKVLVETS |
| | 2019/109051) | YPSQTTRLPPITYTGRFSLEPAPNSGNTLWPEPLFSLVSGLVSMTNPPASSSSAPSPAAS |
| | | SASASQSPPLSCAVPSNDSSPIYSAAPTFPTPNTDIFPEPQSQAFPGSAGTALQYPPPAY |
| | | PAAKGGFQVPMIPDYLFPQQQGDLGLGTPDQKPFQGLESRTQQPSLTPLSTIKAFATQSG |
| | | SQDLKALNTSYQSQLIKPSRMRKYPNRPSKTPPHERPYACPVESCDRRFSRSDELTRHIR |
| | | IHTGQKPFQCRICMRNFSRSDHLTTHIRTHTGEKPFACDICGRKFARSDERKRHTKIHLR |
| | | QKDKKADKSVVASSATSSLSSYPSPVATSYPSPVTTSYPSPATTSYPSPVPTSFSSPGSS |
| | | TYPSPVHSGFPSPSVATTYSSVPPAFPAQVSSFPSSAVTNSFSASTGLSDMTATFSPRTI |
| | | EIC |
| 36 | EGR3 (SEQ ID NO: | MTGKLAEKLPVTMSSLLNQLPDNLYPEEIPSALNLFSGSSDSVVHYNQMATENVMDIGLT |
| | 422 from WO | NEKPNPELSYSGSFQPAPGNKTVTYLGKFAFDSPSNWCQDNIISLMSAGILGVPPASGAL |
| | 2019/109051) | STQTSTASMVQPPQGDVEAMYPALPPYSNCGDLYSEPVSFHDPQGNPGLAYSPQDYQSAK |
| | | PALDSNLFPMIPDYNLYHHPNDMGSIPEHKPFQGMDPIRVNPPPITPLETIKAFKDKQIH |
| | | PGFGSLPQPPLTLKPIRPRKYPNRPSKTPLHERPHACPAEGCDRRFSRSDELTRHLRIHT |
| | | GHKPFQCRICMRSFSRSDHLTTHIRTHTGEKPFACEFCGRKFARSDERKRHAKIHLKQKE |
| | | KKAEKGGAPSASSAPPVSLAPVVTTCA |
| 37 | Synthetic linker | GGSGGGSG |
| 38 | Synthetic linker | GGS GGGS GGGS GGGS G |
| 39 | engineered | LEPGEKP -[YKCPECGKSFS X HQRTH TGEKP]n - YKCPECGKSFS X |
| | transcription factor; | HQRTH - TGKKTS |
| | SEQ ID NO: 147 of | n is an integer from 1-15, e.g., 1, 2, 3, 4, 5, 6, 7, 8, |
| | WO WO2020243651 | 9, 10, 11, 12, 13, 14, or 15, and each X independently |
| | | is a recognition sequence (e.g., a recognition helix) |
| | | capable of binding to 3 bp of a target sequence |
| 40 | transcription factor; | MAPKKKRKVGIHGVPAALEPGEKPYKCPECGKSFSRSDNLVRHQRTHTGEKPYKCPECGK |
| | SEQ ID NO: 99 | SFSHRTTLTNHQRTHTGEKPYKCPECGKSFSREDNLHTHQRTHTGEKPYKCPECGKSFST |
| | FROM WO | SHSLTEHQRTHTGEKPYKCPECGKSFSQSSSLVRHQRTHTGEKPYKCPECGKSFSREDNL |
| | 2020243651 | HTHQRTHTGKKTSKRPAATKKAGQAKKKKGSYPYDVPDYALEEASGSGRADALDDFDLDM |
| | | LGSDALDDFDLDMLGSDALDDFDLDMLGSDALDDFDLDMLINSRSSGSPKKKRKVGSQYL |
| | | PDTDDRHRIEEKRKRTYETFKSIMKKSPFSGPTDPRPPPRRIAVPSRSSASVPKPAPQPY |
| | | PFTSSLSTINYDEFPTMVFPSGQISQASALAPAPPQVLPQAPAPAPAPAMVSALAQAPAP |
| | | VPVLAPGPPQAVAPPAPKPTQAGEGTLSEALLQLQFDDEDLGALLGNSTDPAVFTDLASV |
| | | DNSEFQQLLNQGIPVAPHTTEPMLMEYPEAITRLVTGAQRPPDPAPAPLGAPGLPNGLLS |
| | | GDEDFSSIADMDFSALLGSGSGSRDSREGMFLPKPEAGSAISDVFEGREVCQPKRIRPFH |
| | | PPGSPWANRPLPASLAPTPTGPVHEPVGSLTPAPVPQPLDPAPAVTPEASHLLEDPDEET |
| | | SQAVKALREMADTVIPQKEEAAICGQMDLSHPPPRGHLDELTTTLESMTEDLNLDSPLTP |
| | | ELNEILDTFLNDECLLHAMHISTGLSIFDTSLF |
| 41 | transcription factor; | MAPKKKRKVGIHGVPAALEPGEKPYKCPECGKSFSRSDNLVRHQRTHTGEKPYKCPECGK |
| | SEQ ID NOs: 100 | SFSREDNLHTHQRTHTGEKPYKCPECGKSFSRSDELVRHQRTHTGEKPYKCPECGKSFSQ |
| | FROM WO | SGNLTEHQRTHTGEKPYKCPECGKSFSTSGHLVRHQRTHTGEKPYKCPECGKSFSQNSTL |
| | 2020243651 | TEHQRTHTGKKTSKRPAATKKAGQAKKKKGSYPYDVPDYALEEASGSGRADALDDFDLDM |
| | | LGSDALDDFDLDMLGSDALDDFDLDMLGSDALDDFDLDMLINSRSSGSPKKKRKVGSQYL |
| | | PDTDDRHRIEEKRKRTYETFKSIMKKSPFSGPTDPRPPPRRIAVPSRSSASVPKPAPQPY |
| | | PFTSSLSTINYDEFPTMVFPSGQISQASALAPAPPQVLPQAPAPAPAPAMVSALAQAPAP |
| | | VPVLAPGPPQAVAPPAPKPTQAGEGTLSEALLQLQFDDEDLGALLGNSTDPAVFTDLASV |
| | | DNSEFQQLLNQGIPVAPHTTEPMLMEYPEAITRLVTGAQRPPDPAPAPLGAPGLPNGLLS |
| | | GDEDFSSIADMDFSALLGSGSGSRDSREGMFLPKPEAGSAISDVFEGREVCQPKRIRPFH |
| | | PPGSPWANRPLPASLAPTPTGPVHEPVGSLTPAPVPQPLDPAPAVTPEASHLLEDPDEET |
| | | SQAVKALREMADTVIPQKEEAAICGQMDLSHPPPRGHLDELTTTLESMTEDLNLDSPLTP |
| | | ELNEILDTFLNDECLLHAMHISTGLSIFDTSLF |
| 42 | SEQ ID NO: 101 | MAPKKKRKVGIHGVPAALEPGEKPYKCPECGKSFSRSDNLVRHQRTHTGEKPYKCPECGK |
| | | SFSHRTTLTNHQRTHTGEKPYKCPECGKSFSREDNLHTHQRTHTGEKPYKCPECGKSFST |
| | | SHSLTEHQRTHTGEKPYKCPECGKSFSQSSSLVRHQRTHTGEKPYKCPECGKSFSREDNL |
| | | HTHQRTHTGKKTSKRPAATKKAGQAKKKKGSYPYDVPDYALEDALDDFDLDMLGSDALDD |
| | | FDLDMLGSDALDDFDLDMLGSDALDDFDLDML |
| 43 | SEQ ID NO: 102 | MAPKKKRKVGIHGVPAALEPGEKPYKCPECGKSFSRSDNLVRHQRTHTGEKPYKCPECGK |
| | | SFSREDNLHTHQRTHTGEKPYKCPECGKSFSRSDELVRHQRTHTGEKPYKCPECGKSFSQ |
| | | SGNLTEHQRTHTGEKPYKCPECGKSFSTSGHLVRHQRTHTGEKPYKCPECGKSFSQNSTL |
| | | TEHQRTHTGKKTSKRPAATKKAGQAKKKKGSYPYDVPDYALEDALDDFDLDMLGSDALDD |
| | | FDLDMLGSDALDDFDLDMLGSDALDDFDLDML |
| 44 | SEQ ID NO: 103 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCGGSGGGSGQSQLIKPSRMRKYPNRPSKTPPHERPYACPVESCDRRFSRSDNLVRH |
| | | IRIHTGQKPFQCRICMRNFSHRTTLTNHIRTHTGEKPFACDICGRKFAREDNLHTHTKIH |
| | | LRQKDRPYACPVESCDRRFSTSHSLTEHIRIHTGQKPFQCRICMRNFSQSSSLVRHIRTH |
| | | TGEKPFACDICGRKFAREDNLHTHTKIHLROKDKLEMADHLMLAEGYRLVQRPPSAAAAH |
| | | GPHALRTLPPYAGPGLDSGLRPRGAPLGPPPPRQPGALAYGAFGPPSSFQPFPAVPPPAA |
| | | GIAHLQPVATPYPGRAAAPPNAPGGPPGPQPAPSAAAPPPPAHALGGMDAELIDEEALTS |
| | | LELELGLHRVRELPELFLGQSEFDCFSDLGSAPPAGSVSC |
| 45 | SEQ ID NO: 104 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCGGSGGGSGQSQLIKPSRMRKYPNRPSKTPPHERPYACPVESCDRRFSRSDNLVRH |
| | | IRIHTGQKPFQCRICMRNFSHRTTLTNHIRTHTGEKPFACDICGRKFAREDNLHTHTKIH |
| | | LRQKDRPYACPVESCDRRFSTSHSLTEHIRIHTGQKPFQCRICMRNFSQSSSLVRHIRTH |
| | | TGEKPFACDICGRKFAREDNLHTHTKIHLRQKDK |
| 46 | SEQ ID NO: 105 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCGGSGGGSGQSQLIKPSRMRKYPNRPSKTPPHERPYACPVESCDRRFSRSDNLVRH |
| | | IRIHTGQKPFQCRICMRNFSREDNLHTHIRTHTGEKPFACDICGRKFARSDELVRHTKIH |
| | | LRQKDRPYACPVESCDRRFSQSGNLTEHIRIHTGQKPFQCRICMRNFSTSGHLVRHIRTH |
| | | TGEKPFACDICGRKFAQNSTLTEHTKIHLRQKDKLEMADHLMLAEGYRLVQRPPSAAAAH |
| | | GPHALRTLPPYAGPGLDSGLRPRGAPLGPPPPRQPGALAYGAFGPPSSFQPFPAVPPPAA |
| | | GIAHLQPVATPYPGRAAAPPNAPGGPPGPQPAPSAAAPPPPAHALGGMDAELIDEEALTS |
| | | LELELGLHRVRELPELFLGQSEFDCFSDLGSAPPAGSVSC |
| 47 | SEQ ID NO: 106 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCGGSGGGSGQSQLIKPSRMRKYPNRPSKTPPHERPYACPVESCDRRFSRSDNLVRH |
| | | IRIHTGQKPFQCRICMRNFSREDNLHTHIRTHTGEKPFACDICGRKFARSDELVRHTKIH |
| | | LRQKDRPYACPVESCDRRFSQSGNLTEHIRIHTGQKPFQCRICMRNFSTSGHLVRHIRTH |
| | | TGEKPFACDICGRKFAQNSTLTEHTKIHLRQKDK |
| 48 | SEQ ID NO: 107 | MQSQLIKPSRMRKYPNRPSKTPPHERPYACPVESCDRRFSRSDNLVRHIRIHTGQKPFQC |
| | | RICMRNFSREDNLHTHIRTHTGEKPFACDICGRKFARSDELVRHTKIHLRQKDRPYACPV |
| | | ESCDRRFSQSGNLTEHIRIHTGQKPFQCRICMRNFSTSGHLVRHIRTHTGEKPFACDICG |
| | | RKFAQNSTLTEHTKIHLRQKDKLEMADHLMLAEGYRLVQRPPSAAAAHGPHALRTLPPYA |
| | | GPGLDSGLRPRGAPLGPPPPRQPGALAYGAFGPPSSFQPFPAVPPPAAGIAHLQPVATPY |
| | | PGRAAAPPNAPGGPPGPQPAPSAAAPPPPAHALGGMDAELIDEEALTSLELELGLHRVRE |
| | | LPELFLGQSEFDCFSDLGSAPPAGSVSC |
| 49 | SEQ ID NO: 108 | MQSQLIKPSRMRKYPNRPSKTPPHERPYACPVESCDRRFSRSDNLVRHIRIHTGQKPFQC |
| | | RICMRNFSREDNLHTHIRTHTGEKPFACDICGRKFARSDELVRHTKIHLRQKDRPYACPV |
| | | ESCDRRFSQSGNLTEHIRIHTGQKPFQCRICMRNFSTSGHLVRHIRTHTGEKPFACDICG |
| | | RKFAQNSTLTEHTKIHLRQKDKLEMSGLEMADHMMAMNHGRFPDGTNGLHHHPAHRMGMG |
| | | QFPSPHHHQQQQPQHAFNALMGEHIHYGAGNMNATSGIRHAMGPGTVNGGHPPSALAPAA |
| | | RFNNSQFMGPPVASQGGSLPASMQLQKLNNQYFNHHPYPHNHYMPDLHPAAGHQMNGTNQ |
| | | HFRDCNPKHSGGSSTPGGSGGSSTPGGSGSSSGGGAGSSNSGGGSGSGNMPASVAHVPAA |
| | | MLPPNVIDTDFIDEEVLMSLVIEMGLDRIKELPELWLGQNEFDFMTDFVCKQQPSRVSC |
| 50 | SEQ ID NO: 109 | MSGLEMADHMMAMNHGRFPDGTNGLHHHPAHRMGMGQFPSPHHHQQQQPQHAFNALMGEH |
| | | IHYGAGNMNATSGVRHAMGPGTVNGGHPPSALAPAARFNNSQFMGPPVASQGGSLPASMQ |
| | | LQKLNNQYFNHHPYPHNHYMPDLHPAAGHQMNGTNQHFRDCNPKHSGGSSTPGGSGGSST |
| | | PGGSGSSSGGGAGSSNSGGGSGSGNMPASVAHVPAAMLPPNVIDTDFIDEEVLMSLVIEM |
| | | GLDRIKELPELWLGQNEFDFMTDFVCKQQPSRVSCQSQLIKPSRMRKYPNRPSKTPPHER |
| | | PYACPVESCDRRFSRSDNLVRHIRIHTGQKPFQCRICMRNFSREDNLHTHIRTHTGEKPF |
| | | ACDICGRKFARSDELVRHTKIHLRQKDRPYACPVESCDRRFSQSGNLTEHIRIHTGQKPF |
| | | QCRICMRNFSTSGHLVRHIRTHTGEKPFACDICGRKFAQNSTLTEHTKIHLRQKDK |
| 51 | SEQ ID NO: 110 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCGGSGGGSGRPHACPAEGCDRRFSRSDNLVRHLRIHTGHKPFQCRICMRSFSREDN |
| | | LHTHIRTHTGEKPFACEFCGRKFARSDELVRHAKIHLKQKEHACPAEGCDRRFSQSGNLT |
| | | EHLRIHTGHKPFQCRICMRSFSTSGHLVRHIRTHTGEKPFACEFCGRKFAQNSTLTEHAK |
| | | IHLKQKEKLEMADHLMLAEGYRLVQRPPSAAAAHGPHALRTLPPYAGPGLDSGLRPRGAP |
| | | LGPPPPRQPGALAYGAFGPPSSFQPFPAVPPPAAGIAHLQPVATPYPGRAAAPPNAPGGP |
| | | PGPQPAPSAAAPPPPAHALGGMDAELIDEEALTSLELELGLHRVRELPELFLGQSEFDCF |
| | | SDLGSAPPAGSVSC |
| 52 | SEQ ID NO: 111 | MRPHACPAEGCDRRFSRSDNLVRHLRIHTGHKPFQCRICMRSFSREDNLHTHIRTHTGEK |
| | | PFACEFCGRKFARSDELVRHAKIHLKQKEHACPAEGCDRRFSQSGNLTEHLRIHTGHKPF |
| | | QCRICMRSFSTSGHLVRHIRTHTGEKPFACEFCGRKFAQNSTLTEHAKIHLKQKEKLEMA |
| | | DHLMLAEGYRLVQRPPSAAAAHGPHALRTLPPYAGPGLDSGLRPRGAPLGPPPPRQPGAL |
| | | AYGAFGPPSSFQPFPAVPPPAAGIAHLQPVATPYPGRAAAPPNAPGGPPGPQPAPSAAAP |
| | | PPPAHALGGMDAELIDEEALTSLELELGLHRVRELPELFLGQSEFDCFSDLGSAPPAGSV |
| | | SC |
| 53 | SEQ ID NO: 112 | MRPHACPAEGCDRRFSRSDNLVRHLRIHTGHKPFQCRICMRSFSREDNLHTHIRTHTGEK |
| | | PFACEFCGRKFARSDELVRHAKIHLKQKEHACPAEGCDRRFSQSGNLTEHLRIHTGHKPF |
| | | QCRICMRSFSTSGHLVRHIRTHTGEKPFACEFCGRKFAQNSTLTEHAKIHLKQKEKKAEK |
| | | GGAPSASSAPPVSLAPVVTTCALEMSGLEMADHMMAMNHGRFPDGTNGLHHHPAHRMGMG |
| | | QFPSPHHHQQQQPQHAFNALMGEHIHYGAGNMNATSGIRHAMGPGTVNGGHPPSALAPAA |
| | | RFNNSQFMGPPVASQGGSLPASMQLQKLNNQYFNHHPYPHNHYMPDLHPAAGHQMNGTNQ |
| | | HFRDCNPKHSGGSSTPGGSGGSSTPGGSGSSSGGGAGSSNSGGGSGSGNMPASVAHVPAA |
| | | MLPPNVIDTDFIDEEVLMSLVIEMGLDRIKELPELWLGQNEFDFMTDFVCKQQPSRVSC |
| 54 | SEQ ID NO: 113 | MSGLEMADHMMAMNHGRFPDGTNGLHHHPAHRMGMGQFPSPHHHQQQQPQHAFNALMGEH |
| | | IHYGAGNMNATSGVRHAMGPGTVNGGHPPSALAPAARFNNSQFMGPPVASQGGSLPASMQ |
| | | LQKLNNQYFNHHPYPHNHYMPDLHPAAGHQMNGTNQHFRDCNPKHSGGSSTPGGSGGSST |
| | | PGGSGSSSGGGAGSSNSGGGSGSGNMPASVAHVPAAMLPPNVIDTDFIDEEVLMSLVIEM |
| | | GLDRIKELPELWLGQNEFDFMTDFVCKQQPSRVSCRPHACPAEGCDRRFSRSDNLVRHLR |
| | | IHTGHKPFQCRICMRSFSREDNLHTHIRTHTGEKPFACEFCGRKFARSDELVRHAKIHLK |
| | | QKEHACPAEGCDRRFSQSGNLTEHLRIHTGHKPFQCRICMRSFSTSGHLVRHIRTHTGEK |
| | | PFACEFCGRKFAQNSTLTEHAKIHLKQKEKKAEKGGAPSASSAPPVSLAPVVTTCA |
| 55 | SEQ ID NO: 114 | MTGKLAEKLPVTMSSLLNQLPDNLYPEEIPSALNLFSGSSDSVVHYNQMATENVMDIGLT |
| | | NEKPNPELSYSGSFQPAPGNKTVTYLGKFAFDSPSNWCQDNIISLMSAGILGVPPASGAL |
| | | STQTSTASMVQPPQGDVEAMYPALPPYSNCGDLYSEPVSFHDPQGNPGLAYSPQDYQSAK |
| | | PALDSNLFPMIPDYNLYHHPNDMGSIPEHKPFQGMDPIRVNPPPITPLETIKAFKDKQIH |
| | | PGFGSLPQPPLTLKPIRPRKYPNRPSKTPLHERPHACPAEGCDRRFSRRDELNVHLRIHT |
| | | GHKPFQCRICMRSFSRSDHLTNHIRTHTGEKPFACEFCGRKFARSDDLVRHAKIHLKQKE |
| | | HACPAEGCDRRFSRSDNLVRHLRIHTGHKPFQCRICMRSFSHRTTLTNHIRTHTGEKPFA |
| | | CEFCGRKFAREDNLHTHAKIHLKQKEHACPAEGCDRRFSTSHSLTEHLRIHTGHKPFQCR |
| | | ICMRSFSQSSSLVRHIRTHTGEKPFACEFCGRKFAREDNLHTHAKIHLKQKEKKAEKGGA |
| | | PSASSAPPVSLAPVVTTCA |
| 56 | SEQ ID NO: 115 | MAAAKAEMQLMSPLQISDPFGSFPHSPTMDNYPKLEEMMLLSNGAPQFLGAAGAPEGSGS |
| | | NSSSSSSGGGGGGGGGSNSSSSSSTFNPQADTGEQPYEHLTAESFPDISLNNEKVLVETS |
| | | YPSQTTRLPPITYTGRFSLEPAPNSGNTLWPEPLFSLVSGLVSMTNPPASSSSAPSPAAS |
| | | SASASQSPPLSCAVPSNDSSPIYSAAPTFPTPNTDIFPEPQSQAFPGSAGTALQYPPPAY |
| | | PAAKGGFQVPMIPDYLFPQQQGDLGLGTPDQKPFQGLESRTQQPSLTPLSTIKAFATQSG |
| | | SQDLKALNTSYQSQLIKPSRMRKYPNRPSKTPPHERPYACPVESCDRRFSRSDNLVRHIR |
| | | IHTGQKPFQCRICMRNFSHRTTLTNHIRTHTGEKPFACDICGRKFAREDNLHTHTKIHLR |
| | | QKDRPYACPVESCDRRFSTSHSLTEHIRIHTGQKPFQCRICMRNFSQSSSLVRHIRTHTG |
| | | EKPFACDICGRKFAREDNLHTHTKIHLRQKDKKADKSVVASSATSSLSSYPSPVATSYPS |
| | | PVTTSYPSPATTSYPSPVPTSFSSPGSSTYPSPVHSGFPSPSVATTYSSVPPAFPAQVSS |
| | | FPSSAVTNSFSASTGLSDMTATFSPRTIEIC |
| 57 | SEQ ID NO: 116 | MAAAKAEMQLMSPLQISDPFGSFPHSPTMDNYPKLEEMMLLSNGAPQFLGAAGAPEGSGS |
| | | NSSSSSSGGGGGGGGGSNSSSSSSTFNPQADTGEQPYEHLTAESFPDISLNNEKVLVETS |
| | | YPSQTTRLPPITYTGRFSLEPAPNSGNTLWPEPLFSLVSGLVSMTNPPASSSSAPSPAAS |
| | | SASASQSPPLSCAVPSNDSSPIYSAAPTFPTPNTDIFPEPQSQAFPGSAGTALQYPPPAY |
| | | PAAKGGFQVPMIPDYLFPQQQGDLGLGTPDQKPFQGLESRTQQPSLTPLSTIKAFATQSG |
| | | SQDLKALNTSYQSQLIKPSRMRKYPNRPSKTPPHERPYACPVESCDRRFSRRDELNVHIR |
| | | IHTGQKPFQCRICMRNFSRSDHLTNHIRTHTGEKPFACDICGRKFARSDDLVRHTKIHLR |
| | | QKDRPYACPVESCDRRFSRSDNLVRHIRIHTGQKPFQCRICMRNFSHRTTLTNHIRTHTG |
| | | EKPFACDICGRKFAREDNLHTHTKIHLROKDRPYACPVESCDRRFSTSHSLTEHIRIHTG |
| | | QKPFQCRICMRNFSQSSSLVRHIRTHTGEKPFACDICGRKFAREDNLHTHTKIHLRQKDK |
| | | KADKSVVASSATSSLSSYPSPVATSYPSPVTTSYPSPATTSYPSPVPTSFSSPGSSTYPS |
| | | PVHSGFPSPSVATTYSSVPPAFPAQVSSFPSSAVTNSFSASTGLSDMTATFSPRTIEIC |
| 58 | SEQ ID NO: 117 | MAAAKAEMQLMSPLQISDPFGSFPHSPTMDNYPKLEEMMLLSNGAPQFLGAAGAPEGSGS |
| | | NSSSSSSGGGGGGGGGSNSSSSSSTFNPQADTGEQPYEHLTAESFPDISLNNEKVLVETS |
| | | YPSQTTRLPPITYTGRFSLEPAPNSGNTLWPEPLFSLVSGLVSMTNPPASSSSAPSPAAS |
| | | SASASQSPPLSCAVPSNDSSPIYSAAPTFPTPNTDIFPEPQSQAFPGSAGTALQYPPPAY |
| | | PAAKGGFQVPMIPDYLFPQQQGDLGLGTPDQKPFQGLESRTQQPSLTPLSTIKAFATQSG |
| | | SQDLKALNTSYQSQLIKPSRMRKYPNRPSKTPPHERPYACPVESCDRRFSRSDNLVRHIR |
| | | IHTGQKPFQCRICMRNFSHRTTLTNHIRTHTGEKPFACDICGRKFAREDNLHTHIRTHTG |
| | | EKPFACDICGRKFSTSHSLTEHIRIHTGQKPFQCRICMRNFSQSSSLVRHIRTHTGEKPF |
| | | ACDICGRKFAREDNLHTHTKIHLRQKDKKADKSVVASSATSSLSSYPSPVATSYPSPVTT |
| | | SYPSPATTSYPSPVPTSFSSPGSSTYPSPVHSGFPSPSVATTYSSVPPAFPAQVSSFPSS |
| | | AVTNSFSASTGLSDMTATFSPRTIEIC |
| 59 | SEQ ID NO: 118 | MAAAKAEMQLMSPLQISDPFGSFPHSPTMDNYPKLEEMMLLSNGAPQFLGAAGAPEGSGS |
| | | NSSSSSSGGGGGGGGGSNSSSSSSTFNPQADTGEQPYEHLTAESFPDISLNNEKVLVETS |
| | | YPSQTTRLPPITYTGRFSLEPAPNSGNTLWPEPLFSLVSGLVSMTNPPASSSSAPSPAAS |
| | | SASASQSPPLSCAVPSNDSSPIYSAAPTFPTPNTDIFPEPQSQAFPGSAGTALQYPPPAY |
| | | PAAKGGFQVPMIPDYLFPQQQGDLGLGTPDQKPFQGLESRTQQPSLTPLSTIKAFATQSG |
| | | SQDLKALNTSYQSQLIKPSRMRKYPNRPSKTPPHERPYACPVESCDRRFSRSDNLVRHIR |
| | | IHTGQKPFQCRICMRNFSREDNLHTHIRTHTGEKPFACDICGRKFARSDELVRHTKIHLR |
| | | QKDRPYACPVESCDRRFSQSGNLTEHIRIHTGQKPFQCRICMRNFSTSGHLVRHIRTHTG |
| | | EKPFACDICGRKFAQNSTLTEHTKIHLROKDKKADKSVVASSATSSLSSYPSPVATSYPS |
| | | PVTTSYPSPATTSYPSPVPTSFSSPGSSTYPSPVHSGFPSPSVATTYSSVPPAFPAQVSS |
| | | FPSSAVTNSFSASTGLSDMTATFSPRTIEIC |
| 60 | SEQ ID NO: 119 | MTGKLAEKLPVTMSSLLNQLPDNLYPEEIPSALNLFSGSSDSVVHYNQMATENVMDIGLT |
| | | NEKPNPELSYSGSFQPAPGNKTVTYLGKFAFDSPSNWCQDNIISLMSAGILGVPPASGAL |
| | | STQTSTASMVQPPQGDVEAMYPALPPYSNCGDLYSEPVSFHDPQGNPGLAYSPQDYQSAK |
| | | PALDSNLFPMIPDYNLYHHPNDMGSIPEHKPFQGMDPIRVNPPPITPLETIKAFKDKQIH |
| | | PGFGSLPQPPLTLKPIRPRKYPNRPSKTPLHERPHACPAEGCDRRFSRSDNLVRHLRIHT |
| | | GHKPFQCRICMRSFSHRTTLTNHIRTHTGEKPFACEFCGRKFAREDNLHTHAKIHLKQKE |
| | | HACPAEGCDRRFSTSHSLTEHLRIHTGHKPFQCRICMRSFSQSSSLVRHIRTHTGEKPFA |
| | | CEFCGRKFAREDNLHTHAKIHLKQKEKKAEKGGAPSASSAPPVSLAPVVTTCA |
| 61 | SEQ ID NO: 120 | MAAAKAEMQLMSPLQISDPFGSFPHSPTMDNYPKLEEMMLLSNGAPQFLGAAGAPEGSGS |
| | | NSSSSSSGGGGGGGGGSNSSSSSSTFNPQADTGEQPYEHLTAESFPDISLNNEKVLVETS |
| | | YPSQTTRLPPITYTGRFSLEPAPNSGNTLWPEPLFSLVSGLVSMTNPPASSSSAPSPAAS |
| | | SASASQSPPLSCAVPSNDSSPIYSAAPTFPTPNTDIFPEPQSQAFPGSAGTALQYPPPAY |
| | | PAAKGGFQVPMIPDYLFPQQQGDLGLGTPDQKPFQGLESRTQQPSLTPLSTIKAFATQSG |
| | | SQDLKALNTSYQSQLIKPSRMRKYPNRPSKTPPHERPYACPVESCDRRFSDPGALVRHIR |
| | | IHTGQKPFQCRICMRNFSRSDNLVRHIRTHTGEKPFACDICGRKFAQSGDLRRHTKIHLR |
| | | QKDRPYACPVESCDRRFSTHLDLIRHIRIHTGQKPFQCRICMRNFSTSGNLVRHIRTHTG |
| | | EKPFACDICGRKFARSDNLVRHTKIHLRQKDRPYACPVESCDRRFSQSGHLTEHIRIHTG |
| | | QKPFQCRICMRNFSERSHLREHIRTHTGEKPFACDICGRKFAQAGHLASHTKIHLRQKDK |
| | | KADKSVVASSATSSLSSYPSPVATSYPSPVTTSYPSPATTSYPSPVPTSFSSPGSSTYPS |
| | | PVHSGFPSPSVATTYSSVPPAFPAQVSSFPSSAVTNSFSASTGLSDMTATFSPRTIEIC |
| 62 | SEQ ID NO: 121 | MAAAKAEMQLMSPLQISDPFGSFPHSPTMDNYPKLEEMMLLSNGAPQFLGAAGAPEGSGS |
| | | NSSSSSSGGGGGGGGGSNSSSSSSTFNPQADTGEQPYEHLTAESFPDISLNNEKVLVETS |
| | | YPSQTTRLPPITYTGRFSLEPAPNSGNTLWPEPLFSLVSGLVSMTNPPASSSSAPSPAAS |
| | | SASASQSPPLSCAVPSNDSSPIYSAAPTFPTPNTDIFPEPQSQAFPGSAGTALQYPPPAY |
| | | PAAKGGFQVPMIPDYLFPQQQGDLGLGTPDQKPFQGLESRTQQPSLTPLSTIKAFATQSG |
| | | SQDLKALNTSYQSQLIKPSRMRKYPNRPSKTPPHERPYACPVESCDRRFSRSDNLTRHIR |
| | | IHTGQKPFQCRICMRNFSHSTTLTNHIRTHTGEKPFACDICGRKFARSDNRKTHIRTHTG |
| | | EKPFACDICGRKFSTSHSLTEHIRIHTGQKPFQCRICMRNFSQSSSLTRHIRTHTGEKPF |
| | | ACDICGRKFARSDNRKTHTKIHLRQKDKKADKSVVASSATSSLSSYPSPVATSYPSPVTT |
| | | SYPSPATTSYPSPVPTSFSSPGSSTYPSPVHSGFPSPSVATTYSSVPPAFPAQVSSFPSS |
| | | AVTNSFSASTGLSDMTATFSPRTIEIC |
| 63 | SEQ ID NO: 122 | MTGKLAEKLPVTMSSLLNQLPDNLYPEEIPSALNLFSGSSDSVVHYNQMATENVMDIGLT |
| | | NEKPNPELSYSGSFQPAPGNKTVTYLGKFAFDSPSNWCQDNIISLMSAGILGVPPASGAL |
| | | STQTSTASMVQPPQGDVEAMYPALPPYSNCGDLYSEPVSFHDPQGNPGLAYSPQDYQSAK |
| | | PALDSNLFPMIPDYNLYHHPNDMGSIPEHKPFQGMDPIRVNPPPITPLETIKAFKDKQIH |
| | | PGFGSLPQPPLTLKPIRPRKYPNRPSKTPLHERPHACPAEGCDRRFSRSDNLVRHLRIHT |
| | | GHKPFQCRICMRSFSREDNLHTHIRTHTGEKPFACEFCGRKFARSDELVRHAKIHLKQKE |
| | | HACPAEGCDRRFSQSGNLTEHLRIHTGHKPFQCRICMRSFSTSGHLVRHIRTHTGEKPFA |
| | | CEFCGRKFAQNSTLTEHAKIHLKQKEKKAEKGGAPSASSAPPVSLAPVVTTCA |
| 64 | SEQ ID NO: 123 | MAAAKAEMQLMSPLQISDPFGSFPHSPTMDNYPKLEEMMLLSNGAPQFLGAAGAPEGSGS |
| | | NSSSSSSGGGGGGGGGSNSSSSSSTFNPQADTGEQPYEHLTAESFPDISLNNEKVLVETS |
| | | YPSQTTRLPPITYTGRFSLEPAPNSGNTLWPEPLFSLVSGLVSMTNPPASSSSAPSPAAS |
| | | SASASQSPPLSCAVPSNDSSPIYSAAPTFPTPNTDIFPEPQSQAFPGSAGTALQYPPPAY |
| | | PAAKGGFQVPMIPDYLFPQQQGDLGLGTPDQKPFQGLESRTQQPSLTPLSTIKAFATQSG |
| | | SQDLKALNTSYQSQLIKPSRMRKYPNRPSKTPPHERPYACPVESCDRRFSRSDNLTRHIR |
| | | IHTGQKPFQCRICMRNFSRSDNLTTHIRTHTGEKPFACDICGRKFARSDERKRHIRTHTG |
| | | EKPFACDICGRKFSQSGNLTEHIRIHTGQKPFQCRICMRNFSTSGHLTRHIRTHTGEKPF |
| | | ACDICGRKFAQSSTRKEHTKIHLRQKDKKADKSVVASSATSSLSSYPSPVATSYPSPVTT |
| | | SYPSPATTSYPSPVPTSFSSPGSSTYPSPVHSGFPSPSVATTYSSVPPAFPAQVSSFPSS |
| | | AVTNSFSASTGLSDMTATFSPRTIEIC |
| 65 | SEQ ID NO: 124 | MAAAKAEMQLMSPLQISDPFGSFPHSPTMDNYPKLEEMMLLSNGAPQFLGAAGAPEGSGS |
| | | NSSSSSSGGGGGGGGGSNSSSSSSTFNPQADTGEQPYEHLTAESFPDISLNNEKVLVETS |
| | | YPSQTTRLPPITYTGRFSLEPAPNSGNTLWPEPLFSLVSGLVSMTNPPASSSSAPSPAAS |
| | | SASASQSPPLSCAVPSNDSSPIYSAAPTFPTPNTDIFPEPQSQAFPGSAGTALQYPPPAY |
| | | PAAKGGFQVPMIPDYLFPQQQGDLGLGTPDQKPFQGLESRTQQPSLTPLSTIKAFATQSG |
| | | SQDLKALNTSYQSQLIKPSRMRKYPNRPSKTPPHERPYACPVESCDRRFSRSDNLVRHIR |
| | | IHTGQKPFQCRICMRNFSREDNLHTHIRTHTGEKPFACDICGRKFARSDELVRHIRTHTG |
| | | EKPFACDICGRKFSQSGNLTEHIRIHTGQKPFQCRICMRNFSTSGHLVRHIRTHTGEKPF |
| | | ACDICGRKFAQNSTLTEHTKIHLRQKDKKADKSVVASSATSSLSSYPSPVATSYPSPVTT |
| | | SYPSPATTSYPSPVPTSFSSPGSSTYPSPVHSGFPSPSVATTYSSVPPAFPAQVSSFPSS |
| | | AVTNSFSASTGLSDMTATFSPRTIEIC |
| 66 | SEQ ID NO: 125 | MAPKKKRKVGIHGVPAALEPGEKPYKCPECGKSFSSPADLTRHQRTHTGEKPYKCPECGK |
| | | SFSRSDNLVRHQRTHTGEKPYKCPECGKSFSREDNLHTHQRTHTGEKPYKCPECGKSFSR |
| | | SDELVRHQRTHTGEKPYKCPECGKSFSQSGNLTEHQRTHTGEKPYKCPECGKSFSTSGHL |
| | | VRHQRTHTGKKTSKRPAATKKAGQAKKKKGSYPYDVPDYALEDALDDFDLDMLGSDALDD |
| | | FDLDMLGSDALDDFDLDMLGSDALDDFDLDML |
| 67 | SEQ ID NO: 126 | MAPKKKRKVGIHGVPAALEPGEKPYKCPECGKSFSDPGALVRHQRTHTGEKPYKCPECGK |
| | | SFSRSDNLVRHQRTHTGEKPYKCPECGKSFSQSGDLRRHQRTHTGEKPYKCPECGKSFST |
| | | HLDLIRHQRTHTGEKPYKCPECGKSFSTSGNLVRHQRTHTGEKPYKCPECGKSFSRSDNL |
| | | VRHQRTHTGKKTSKRPAATKKAGQAKKKKGSYPYDVPDYALEDALDDFDLDMLGSDALDD |
| | | FDLDMLGSDALDDFDLDMLGSDALDDFDLDML |
| 68 | SEQ ID NO: 127 | MAPKKKRKVGIHGVPAALEPGEKPYKCPECGKSFSRSDNLVRHQRTHTGEKPYKCPECGK |
| | | SFSREDNLHTHQRTHTGEKPYKCPECGKSFSRSDELVRHQRTHTGEKPYKCPECGKSFSQ |
| | | SGNLTEHQRTHTGEKPYKCPECGKSFSTSGHLVRHQRTHTGEKPYKCPECGKSFSQNSTL |
| | | TEHQRTHTGKKTSKRPAATKKAGQAKKKKGSDALDDFDLDMLGSDALDDFDLDMLGSDAL |
| | | DDFDLDMLGSDALDDFDLDML |
| 69 | SEQ ID NO: 128 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCQSQLIKPSRMRKYPNRPSKTPPHERPYACPVESCDRRFSRSDNLVRHIRIHTGQK |
| | | PFQCRICMRNFSREDNLHTHIRTHTGEKPFACDICGRKFARSDELVRHTKIHLRQKDRPY |
| | | ACPVESCDRRFSQSGNLTEHIRIHTGQKPFQCRICMRNFSTSGHLVRHIRTHTGEKPFAC |
| | | DICGRKFAQNSTLTEHTKIHLRQKDK |
| 70 | SEQ ID NO: 129 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCGGSGGGSGGGSGGGSGQSQLIKPSRMRKYPNRPSKTPPHERPYACPVESCDRRFS |
| | | RSDNLVRHIRIHTGQKPFQCRICMRNFSREDNLHTHIRTHTGEKPFACDICGRKFARSDE |
| | | LVRHTKIHLRQKDRPYACPVESCDRRFSQSGNLTEHIRIHTGQKPFQCRICMRNFSTSGH |
| | | LVRHIRTHTGEKPFACDICGRKFAQNSTLTEHTKIHLRQKDK |
| 71 | SEQ ID NO: 130 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCADHLMLAEGYRLVQRPPSAAAAHGPHALRTLPPYAGPGLDSGLRPRGAPLGPPPP |
| | | RQPGALAYGAFGPPSSFQPFPAVPPPAAGIAHLQPVATPYPGRAAAPPNAPGGPPGPQPA |
| | | PSAAAPPPPAHALGGMDAELIDEEALTSLELELGLHRVRELPELFLGQSEFDCFSDLGSA |
| | | PPAGSVSCQSQLIKPSRMRKYPNRPSKTPPHERPYACPVESCDRRFSRSDNLVRHIRIHT |
| | | GQKPFQCRICMRNFSREDNLHTHIRTHTGEKPFACDICGRKFARSDELVRHTKIHLRQKD |
| | | RPYACPVESCDRRFSQSGNLTEHIRIHTGQKPFQCRICMRNFSTSGHLVRHIRTHTGEKP |
| | | FACDICGRKFAQNSTLTEHTKIHLRQKDK |
| 72 | SEQ ID NO: 131 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCGGSGGGSGADHLMLAEGYRLVQRPPSAAAAHGPHALRTLPPYAGPGLDSGLRPRG |
| | | APLGPPPPRQPGALAYGAFGPPSSFQPFPAVPPPAAGIAHLQPVATPYPGRAAAPPNAPG |
| | | GPPGPQPAPSAAAPPPPAHALGGMDAELIDEEALTSLELELGLHRVRELPELFLGQSEFD |
| | | CFSDLGSAPPAGSVSCGGSGGGSGQSQLIKPSRMRKYPNRPSKTPPHERPYACPVESCDR |
| | | RFSRSDNLVRHIRIHTGQKPFQCRICMRNFSREDNLHTHIRTHTGEKPFACDICGRKFAR |
| | | SDELVRHTKIHLRQKDRPYACPVESCDRRFSQSGNLTEHIRIHTGQKPFQCRICMRNFST |
| | | SGHLVRHIRTHTGEKPFACDICGRKFAQNSTLTEHTKIHLRQKDK |
| 73 | SEQ ID NO: 205 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCGGSGGGSGGGSGGGSGQSQLIKPSRMRKYPNRPSKTPPHERPYACPVESCDRRFS |
| | | RSDNLVRHIRIHTGQKPFQCRICMRNFSREDNLHTHIRTHTGEKPFACDICGRKFARSDE |
| | | LVRHTKIHLRQKDRPYACPVESCDRRFSQSGNLTEHIRIHTGQKPFQCRICMRNFSTSGH |
| | | LVRHIRTHTGEKPFACDICGRKFAQNSTLTEHTKIHLROKDK |
| 74 | SEQ ID NO: 207 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCADHLMLAEGYRLVQRPPSAAAAHGPHALRTLPPYAGPGLDSGLRPRGAPLGPPPP |
| | | RQPGALAYGAFGPPSSFQPFPAVPPPAAGIAHLQPVATPYPGRAAAPPNAPGGPPGPQPA |
| | | PSAAAPPPPAHALGGMDAELIDEEALTSLELELGLHRVRELPELFLGQSEFDCFSDLGSA |
| | | PPAGSVSCQSQLIKPSRMRKYPNRPSKTPPHERPYACPVESCDRRFSRSDNLVRHIRIHT |
| | | GQKPFQCRICMRNFSREDNLHTHIRTHTGEKPFACDICGRKFARSDELVRHTKIHLRQKD |
| | | RPYACPVESCDRRFSQSGNLTEHIRIHTGQKPFQCRICMRNFSTSGHLVRHIRTHTGEKP |
| | | FACDICGRKFAQNSTLTEHTKIHLRQKDK |
| 75 | SEQ ID NO: 209 | MAADHLMLAEGYRLVQRPPSAAAAHGPHALRTLPPYAGPGLDSGLRPRGAPLGPPPPRQP |
| | | GALAYGAFGPPSSFQPFPAVPPPAAGIAHLQPVATPYPGRAAAPPNAPGGPPGPQPAPSA |
| | | AAPPPPAHALGGMDAELIDEEALTSLELELGLHRVRELPELFLGQSEFDCFSDLGSAPPA |
| | | GSVSCGGSGGGSGADHLMLAEGYRLVQRPPSAAAAHGPHALRTLPPYAGPGLDSGLRPRG |
| | | APLGPPPPRQPGALAYGAFGPPSSFQPFPAVPPPAAGIAHLQPVATPYPGRAAAPPNAPG |
| | | GPPGPQPAPSAAAPPPPAHALGGMDAELIDEEALTSLELELGLHRVRELPELFLGQSEFD |
| | | CFSDLGSAPPAGSVSCGGSGGGSGQSQLIKPSRMRKYPNRPSKTPPHERPYACPVESCDR |
| | | RFSRSDNLVRHIRIHTGQKPFQCRICMRNFSREDNLHTHIRTHTGEKPFACDICGRKFAR |
| | | SDELVRHTKIHLRQKDRPYACPVESCDRRFSQSGNLTEHIRIHTGQKPFQCRICMRNFST |
| | | SGHLVRHIRTHTGEKPFACDICGRKFAQNSTLTEHTKIHLRQKDK |
| 76 | SEQ ID NO: 213 | MELELDAGDQDLLAFL |
| 77 | SEQ ID NO: 217 | MELELDAGDQDLLAFLLEESGDLGTAPDEAVRAPLDWALPLSEVPSDWEVDDLLCSLLSP |
| | | PASLNILSSSNPCLVHHDHTYSLPRETVSMDLESESCRKEGTQMTPQHMEELAEQEIARL |
| | | VLTDEEKSLLEKEGLILPETLPLTKTEEQILKRVRRPYACPVESCDRRFSRSDNLVRHIR |
| | | IHTGQKPFQCRICMRNFSREDNLHTHIRTHTGEKPFACDICGRKFARSDELVRHTKIHLR |
| | | QKDRPYACPVESCDRRFSQSGNLTEHIRIHTGQKPFQCRICMRNFSTSGHLVRHIRTHTG |
| | | EKPFACDICGRKFAQNSTLTEHTKIHLRQKDVYVGGLESRVLKYTAQNMELQNKVQLLEE |
| | | QNLSLLDQLRKLQAMVIEISNKTSSSSTCILVLLVSFCLLLVPAMYSSDTRGSLPAEHGV |
| | | LSRQLRALPSEDPYQLELPALQSEVPKDSTHQWLDGSDCVLQAPGNTSCLLHYMPQAPSA |
| | | EPPLEWPFPDLFSEPLCRGPILPLQANLTRKGGWLPTGSPSVILQDRYSG |
| 78 | SEQ ID NO: 219 | MELELDAGDQDLLAFLLEESGDLGTAPDEAVRAPLDWALPLSEVPSDWEVDDLLCSLLSP |
| | | PASLNILSSSNPCLVHHDHTYSLPRETVSMDLESESCRKEGTQMTPQHMEELAEQEIARL |
| | | VLTDEEKSLLEKEGLILPETLPLTKTEEQILKRVRRPYACPVESCDRRFSRSDNLVRHIR |
| | | IHTGQKPFQCRICMRNFSHRTTLTNHIRTHTGEKPFACDICGRKFAREDNLHTHTKIHLR |
| | | QKDRPYACPVESCDRRFSTSHSLTEHIRIHTGQKPFQCRICMRNFSQSSSLVRHIRTHTG |
| | | EKPFACDICGRKFAREDNLHTHTKIHLRQKDVYVGGLESRVLKYTAQNMELQNKVQLLEE |
| | | QNLSLLDQLRKLQAMVIEISNKTSSSSTCILVLLVSFCLLLVPAMYSSDTRGSLPAEHGV |
| | | LSRQLRALPSEDPYQLELPALQSEVPKDSTHQWLDGSDCVLQAPGNTSCLLHYMPQAPSA |
| | | EPPLEWPFPDLFSEPLCRGPILPLQANLTRKGGWLPTGSPSVILQDRYSG |
| 79 | SEQ ID NO: 221 | MELELDAGDQDLLAFLLEESGDLGTAPDEAVRAPLDWALPLSEVPSDWEVDDLLCSLLSP |
| | | PASLNILSSSNPCLVHHDHTYSLPRETVSMDLESESCRKEGTQMTPQHMEELAEQEIARL |
| | | VLTDEEKSLLEKEGLILPETLPLTKTEEQILKRVRLEPGEKPYKCPECGKSFSRSDNLVR |
| | | HQRTHTGEKPYKCPECGKSFSREDNLHTHQRTHTGEKPYKCPECGKSFSRSDELVRHQRT |
| | | HTGEKPYKCPECGKSFSQSGNLTEHQRTHTGEKPYKCPECGKSFSTSGHLVRHQRTHTGE |
| | | KPYKCPECGKSFSQNSTLTEHQRTHTGKKTSVYVGGLESRVLKYTAQNMELQNKVQLLEE |
| | | QNLSLLDQLRKLQAMVIEIS |
| 80 | SEQ ID NO: 223 | MELELDAGDQDLLAFLLEESGDLGTAPDEAVRAPLDWALPLSEVPSDWEVDDLLCSLLSP |
| | | PASLNILSSSNPCLVHHDHTYSLPRETVSMDLESESCRKEGTQMTPQHMEELAEQEIARL |
| | | VLTDEEKSLLEKEGLILPETLPLTKTEEQILKRVRRPYACPVESCDRRFSRSDNLVRHIR |
| | | IHTGQKPFQCRICMRNFSREDNLHTHIRTHTGEKPFACDICGRKFARSDELVRHTKIHLR |
| | | QKDRPYACPVESCDRRFSQSGNLTEHIRIHTGQKPFQCRICMRNFSTSGHLVRHIRTHTG |
| | | EKPFACDICGRKFAQNSTLTEHTKIHLRQKDVYVGGLESRVLKYTAQNMELQNKVQLLEE |
| | | QNLSLLDQLRKLQAMVIEIS |
| 81 | Regulatory element; | ggaggaagccatcaactaaactacaatgactgtaagatacaaaattgggaatggtaacat |
| | SEQ ID NOs: 1 | attttgaagttctgttgacataaagaatcatgatattaatgcccatggaaatgaaagggc |
| | FROM WO | gatcaacactatggtttgaaaagggggaaattgtagagcacagatgtgttcgtgtggcag |
| | 2018187363 | tgtgctgtctctagcaatactcagagaagagagagaacaatgaaattctgattggcccca |
| | (Homo sapiens) | gtgtgagcccagatgaggttcagctgccaactttctctttcacatcttatgaaagtcatt |
| | | taagcacaactaactttttttttttttttttttttttgagacagagtcttgctctgttgc |
| | | ccaggacagagtgcagtagtgactcaatctcggctcactgcagcctccacctcctaggct |
| | | caaacggtcctcctgcatcagcctcccaagtagctggaattacaggagtggcccaccatg |
| | | cccagctaatttttgtatttttaatagatacgggggtttcaccatatcacccaggctggt |
| | | ctcgaactcctggcctcaagtgatccacctgcctcggcctcccaaagtgctgggattata |
| | | ggcgtcagccactatgcccaacccgaccaaccttttttaaaataaatatttaaaaaattg |
| | | gtatttcacatatatactagt |
| 82 | SEQ ID NO: 2 | agtttggacaagaactatagttctagctttctctgggtctccaccttgcagagaatgcag |
| | (Mus musculus) | ctttcattatctcatgagccaaactctcatcatctctttccatatatctgtcggtgctct |
| | | tccatgagtactctaacacacacagaaggagcacttacacaggctgttgtttttctctta |
| | | ttatcatagctgttgttcagacatgtgcattctgttcttgttgcttcaatgctaaaggag |
| | | tctcaggatatgagaactgtaccagccgaggcatcaggaaacatgggtggaaattcccac |
| | | agtactatttgttcactgtgtgaccttgggccagtcacatccctttcctgaggcttcgat |
| | | tccccaagctataaaagaagcatctcttaacctttttttaggtcatgagtcaggcccagc |
| | | acactctcagggagactcatgagagtacagatcatttcccatagaaaaaccatagtttta |
| | | tatccagaggcttttctgtaag |
| 83 | SEQ ID NO: 3 | ggttccagttcagaggcagagcatttggggttcccagtcaggagctttcctctctccgct |
| | (Mus musculus) | ccttagtttcctctctttaaaaaaaaatgggtgatagtatagaaaggaagctctgggctc |
| | | ggggaccagggccctgggatccccgctcccagccactcgctcctgacccttccagggaca |
| | | agctcccccccaccccgtcctttccaggctgccactagaagagatggggacgcgtggtca |
| | | gccgcttctgtcgccccccagggaacggtctcacgctggagggggcagtgccctcggaac |
| | | aggacagtcagcccaagccagccaagcgcgcgcggacgtccttcaccgcagagcaattgc |
| | | aggtaccccgggcaagccccgaagcgtgtgggcggggcttcggagtgggcgtggttgttc |
| | | gggacttgtgactccgccccttgtgcggggacccgcgtgaggccgctccaaggatgaagc |
| | | tgcctggggcgtggcctcggaccctgagcctctgattgggcggaggtctcagggcccttc |
| | | tgcgccccacaggttatgcaggcgcagttcgcgcaggacaacaacccggacgcgcagacg |
| | | ctgcagaagctggcggacatgacgggcctcagtcgcagggtcatccaggtggggctccgg |
| | | ggtctcggccttcaggtctagggtgaaccttagggaagcgctgaagctcgtagtggtacg |
| | | gatggtcgcgcgtgcacgtggccgcccctctccagtgtggcctaaggaccccagtcggca |
| | | cgggttgacccttttccttgattactgagagtgcagaggctgt |
| 84 | SEQ ID NO: 4 | tggtgggaagacatgtccagggaagaaatggcctccagaggcctgaggtggggaaatgct |
| | (Mus musculus) | ggaggtggagagaggaacaactgactgaaaatgagcttccactgtggcttagtagcctat |
| | | accaagtctagagtatagggtaggagaagattaggaaagcgatgggtctgagaatgatgt |
| | | ggcctgttgacttttgtaaacccaaagcaccttggactaaaccctatgaacagtgtggtg |
| | | ccaccaaagactataatgagctcagggaacagaattctgtgtgcatggtgattttttttt |
| | | tttttttctgctaactgcagtctgggtgatgcattgacaaaccaatcctggaaagtaaga |
| | | ggcaagggcagctgggacggtgagaggagcctgatgggaaccaggccaagcagggcagca |
| | | gaggcgatgaagaggatgtggtgcatccagagactcacttcattagctggaggcactgct |
| | | ggatagggtctgaaggttctggtatctgagttggcgggctgggtgagtggtggctctgct |
| | | tcctgaacagtgtgtgcaagaggaaacagggttaagggctaggacagtcacaggtgagtc |
| | | agcctcacaagagcaaccttcccctagtgcaga |
| 85 | SEQ ID NO: 5 | ggaggtctcc ttttgccccg gttccaacaa gagaatgcaa ggctgtatct caatttcctt 60 |
| | (Mus musculus) | gagcctctct gtattataga agaaaagtag ggaagccata cgccccttct gagcttcagt 120 |
| | | gtctctctgt ctctgcaaat gaggctgggg aggctggggg cgggcgtgaa agaggcccgc 180 |
| | | gccaagccga cccccacctc tgccccctcc ccaggtcaac aacctcatct ggcacgtgcg 240 |
| | | gtgcctcgag tgctccgtgt gtcgcacatc gctgaggcag cagaatagct gctacatcaa 300 |
| | | gaacaaggag atctactgca agatggacta cttcaggtag gcagcggcca tcccgccagc 360 |
| | | aagcgctgga gcatgaacgc cttgcacacg cgtgcctagg ccacttgtgt ggcctgtgct 420 |
| | | ctccaattcc tgagccctgc tgttcagagt gcacaacgcg gctcagcgca ctggcccggc 480 |
| | | cctcctactc agcacgtctt acacagaagg gagcgccagt ctcagcctga gttctggcgg 540 |
| | | gggatctgcc tcgggttcct ccgatctgac aggcgctggc cacgggtctg gttccatctc 600 |
| | | tggtcttttc tggccccgag caccagtgtg ttctgttgag ctctgatgtc cgaggctctg 660 |
| | | gcccggatca 670 |
| 86 | SEQ ID NO: 6 | ctctggctac ctcttatctt gggcattcac gacaatttct aattgcaggt agtttgtgtg 60 |
| | (Mus musculus) | tgtgcgcgtg ttttttttcc ccctcagagg cttggattgc aaaggaacta agcgattact 120 |
| | | tcaagagcca cgggttaagt gcagggagag ggggagagag agggaaaaaa acccaatcca 180 |
| | | aattcaaatt gcttcattag agagacaccg cttttgtggg gaagggcttt aaatgcccac 240 |
| | | tacaaagtta ggactcattg ttcagcgccg gtttatataa caggcgaggg gaggcgctgg 300 |
| | | gctctgacag ctccgagcca gttcagcagc cgccgtcgcc tgcattccct ccccctcccc 360 |
| | | caggtgatgg cccagccagg gtccggctgc aaagcgacca cccgctgtct cgaagggacc 420 |
| | | gctccgcctg ccatggtgag tcctttcggt cctgctttcg gccccgagtc cccccaacag 480 |
| | | cacaggccag ggcttctggc tcagccttcc ggctaccaac ctctacccct gcgctggaaa 540 |
| | | actgccgata ggagccgcct ctcgttgagc cttggttttt ctggcctgga atgtgagctt 600 |
| | | tggctgcttc ctgcacccag gatgcgctgt gttaaaagtt gggggccgtc ccttcttctc 660 |
| | | caataggtcc tttcattctt gtactccagc ctagggcgcg acatccctgg cacatttcgg 720 |
| | | tgtcagtcgg tgcgcgagga aaccagattc aactctgagt actcggctaa gcgcttcgct 780 |
| | | gttcctctct cccatttcag gctcagtcag acgcagaggc cttggcaggc gctctggaca 840 |
| | | aggacgaagg tagagcctcc ccatgtacgc ccagcacacc gtctgtctgc tcgccgccct 900 |
| | | ctgctgcctc ttccgtgccg tctgccggca agaatatctg ctccagttgc ggtctggaga 960 |
| | | tcctggaccg gtatctgctc aaggtgagtc agggtaggtg tgcctgcttg cccacgggtg 1020 |
| | | tggtttgcag ccccaagagc tgt 1043 |
| 87 | SEQ ID NO: 7 | caagactttt aaaagtttag ataaataaac aaacatttga cggctttcca tcacatctag 60 |
| | (Mus musculus) | actataatcc aaagatctat atggtcccaa acgacttaca cttaactacc gtctcccata 120 |
| | | tggcttcttc ccccatcagt cattgtcctc agccatagtg gcctccctgt tcctttgggt 180 |
| | | acaagggaac aactccctga gaggttccat tagctgctgt tgcctgagat gctcttgagc 240 |
| | | ccacaccatc tgctcatttc tctcctcacg tgtcagtgat taagaggctg tccttggcct 300 |
| | | cccgtcaaaa ttacatccct gccgctttcc acttcttgcc ttcttatttt ctaaatagaa 360 |
| | | ctaactcacc actacccaac attctatata attggatatc tgtcctctgt ttaaatataa 420 |
| | | tgttgacttc aagaaagaac gttgtcactg ccctgtcacc agacttttaa acagtgccta 480 |
| | | tcgtgtggca catgctcagt gaaattg 507 |
| 88 | SEQ ID NO: 8 | tcaacagggg gacacttggg aaagaaggat ggggacagag ccgagaggac tgttacacat 60 |
| | (Mus musculus) | tagagaaaca tcagtgactg tgccagcttt ggggtagact gcacaaaagc cctgaggcag 120 |
| | | cacaggcagg atccagtctg ctggtcccag gaagctaacc gtctcagaca gagcacaaag 180 |
| | | caccgagaca tgtgccacaa ggcttgtgta gagaggtcag aggacagcgt acaggtccca 240 |
| | | gagatcaaac tcaacctcac caggcttggc agcaagcctt taccaaccca cccccacccc 300 |
| | | acccaccctg cacgcgcccc tctcccctcc ccatggtctc ccatggctat ctcacttggc 360 |
| | | cctaaaatgt ttaaggatga cactggctgc tgagtggaaa tgagacagca gaagtcaaca 420 |
| | | gtagatttta ggaaagccag agaaaaaggc ttgtgctgtt tttagaaagc caagggacaa 480 |
| | | gctaagatag ggcccaagta at 502 |
| 89 | SEQ ID NO: 9 | aaatagaact gtgagatagg gggagagggg gcaggaagga caagagaccc ctgtctcatt 60 |
| | (Mus musculus) | gtgatcccca cctgtctgct ctgtgggagg gtacccatga gggccagccc acagccctta 120 |
| | | ggtggacatt gtctggtcct gtctcactgt ccctcccagc agccccagag gccaggagac 180 |
| | | aggggtctca gtcctcactg agagatgtgt aaactgaggc ccagtgaatg ttgagggcca 240 |
| | | gggcatgccc ttggtgggat gtgacctggg tctccttcgc acgggcttcc tccccgaagc 300 |
| | | cgagctgagc atttggagtt tgaaatgttt ccgtacttag caatctgctc ctctattccc 360 |
| | | gggcggactt ccgatagctc cggccttatg ctgcactaga taagatggag cagggagagg 420 |
| | | acacggcact acttatgtaa ccggcctctt gaaaaatgga gcagcggtca gggcggaaca 480 |
| | | agacgtcctc tctctacgca tccctctcct ttccctgcta aggctgcagc tggagtcaga 540 |
| | | ggcagggctg ttccaatctg tctttgatca gtaacgcagc cagcctccag cctccgtcag 600 |
| | | cctcctcatg gctgagaccc ggcctcagtt tcccccactt acatcccgag gatcagagcc 660 |
| | | tgtgaggatg aaatgggata aggtagctgg aaccgtctgg cagagagcga gtcctcagga 720 |
| | | ctgttgatgc ctgtggctgc ctggcttgac cccaagtgac cccgcctcct catcctgcag 780 |
| | | caggagaa 788 |
| 90 | SEQ ID NO: 10 | tctatagaat gtgtccccag ccttgttttc cacacttgat acgcaaggaa tgcataccac 60 |
| | (Mus musculus) | agagagggat gagggtagca tccagcctgc ttcctgtgtg tcggggcgct acagccacat 120 |
| | | ctccccagtc catctcagac cgtcacagag cttcgccgaa tgtatagctt tgttctctgt 180 |
| | | gcagacaggg agacagagcc ttgggaagca taggtgcttg cttctttgcc cactgagtct 240 |
| | | tagctggact tgcacaccac atgcctcaca gccgggcgca cttgcatttg tcacccaggc 300 |
| | | ccagtgatga tggctctgct tgctttgtgc tttgtgccaa ctacagctcc agcacctgtg 360 |
| | | ccctgggttt tcactccttt agttgaacac gtagttactg gggttgtagg gatggagcct 420 |
| | | ttctgcttcc ttctggcaaa gtccttagcg gcctgctgcg ggggtggggg gtgttcaggg 480 |
| | | gagtggtgat gaagtatgac ag 502 |
| 91 | SEQ ID NO: 11 | tctccagttg gagaaacaga tgctgtaact ggggccacag tataaagaga gcccagacat 60 |
| | (Mus musculus) | tgaactgtca acacagaagc ctggcacact ggaactggca gtccagctgg gaacaagggg 120 |
| | | tagaggctga ggccactaag tcaactgagg caggagacat aggagctaaa gcagctgaag 180 |
| | | ggtgcaggac agctgggggg tctgaagtgg gcctcatgcc cagagctatg aagtcagggg 240 |
| | | ctgtagccta ggagccttgg aagccagctg gcaagctgtg gcccaaagac gctgactcac 300 |
| | | caggaggggg cagctggagc caggcactcc taaggtttcc aggaagggca gccttccagg 360 |
| | | gctcagctag gggagacagt gttgacagca agttgtcagg caacttgagc tactgggcag 420 |
| | | ctgggaagct gtcccttggt ccccagtatc atcatcaccc cagacgctgc ccacctgcct 480 |
| | | caggtcccac acagtgatcc tcccatcttt aacacaacac atgaccagag aga 533 |
| 92 | SEQ ID NO: 12 | gtcaccctcc ccccaaacaa ccccttcttc tctggttcga gaaattacag gcatgaaaga 60 |
| | (Mus musculus) | tataaatcgg gatgcttgac ttgggaatat aaatcactaa agcttggggg caggggtggg 120 |
| | | cgacctttgt gaccgtcctt gtgcgtgcca gtaaatcctg tggtccaggg gagaagaaaa 180 |
| | | ggctgtgtgg cttctgctca caaagctgca gaaaccattc tttaagccca aaagcacttc 240 |
| | | cagagagagc agagcatccc caggctgctg gctcagcaag ttcactgtgc tcaatctcag 300 |
| | | gaagtgagga taagagcagt gcctggagag tgcctggtgc tgagctgagg gtttctgaac 360 |
| | | acattaaagc ggggagcatg gaccgggcct caggaggggt gttgaacatc cctaggcaga 420 |
| | | ggagtctagc ttcctgggaa aagatatcag gttaagcaca cacatgtcct ctggaataag 480 |
| | | ataatctttc tgatcacaca ctatacacac acaaaagcct gctc 524 |
| 93 | SEQ ID NO: 13 | gccctctagg ccacctgacc aggtcccctc agtccccccc ttcccacact cccacactca 60 |
| | (mus musculus) | gcccccctcc cccccccccg acccctgcag gattatcctg tctgtgttcc tgactcagcc 120 |
| | | tgggagccac ctgggcagca ggggccaagg gtgtcctaga agggacctgg agtccacgct 180 |
| | | gggccaagcc tgccctttct ccctctgtct tccgtccctg cttgcggttc tgctgaatgt 240 |
| | | ggttatttct ctggctcctt ttacagagaa tgctgctgct aattttatgt ggagctctga 300 |
| | | ggcagtgtaa ttggaagcca gacaccctgt cagcagtggg ctcccgtcct gagctgccat 360 |
| | | gcttcctgct ctcctcccgt cccggctcct catttcatgc agccacctgt cccagggaga 420 |
| | | gaggagtcac ccaggcccct cagtccgccc cttaaataag aaagcctccg ttgctcggca 480 |
| | | cacataccaa gcagccgctg gtgcaatct 509 |
| 94 | SEQ ID NO: 14 | gtgttcttcc cttccccttt ggacccccga gacaagccaa taaaatactc ggcagggtgg 60 |
| | (Mus musculus) | cttctctcct ttttttgcca gtaataaaca gactcagagc aagttaaggg tctggtccaa 120 |
| | | ggtcatggct gggatcagtg acagagccca gaagagaacc tgagacttct tgctgagcca 180 |
| | | agctggagag gacagaaagg aatgcgtcta ctccatgcat gaccctctgc cagctttgct 240 |
| | | ccttcctaag ggaccatgaa cgatatgtgc acaccgctca tacgtatgtg cacacctgca 300 |
| | | agaggaggca tcccatgtac acctatgaga cgcacagaga aacatatatg tagccatagg 360 |
| | | ctagaaattc tttctctttc taggtctgcc cctctgca 398 |
| 95 | SEQ ID NO: 15 | ggaccactca gtgtacacgg aatgtagaat tgagtctgcc attggtcttc cctcaaagtc 60 |
| | (Mus musculus) | ttggaggctt gggactgata ttgggagcat ctgggcagag aaggccacaa agacagggtg 120 |
| | | gtttttctac actgggacat actcgtgagc atgcacagag gcgtgtcccc aacttccctg 180 |
| | | tcacccctgt cctctgccgg ctagagggga tgcgggggtg gacatatgct gctattgggc 240 |
| | | agatatcaca tgttaagagg tggggggggg ctcaagaggc ggagggctag gagcatccca 300 |
| | | tggggagagg ttctggtttt cttgctgcct ctagctgcta taaatacgtt agcacttgag 360 |
| | | caactggaaa gctctgagta atttaggatg cacaaagctg taatttaact ccagcatctc 420 |
| | | agtgtgcgag agcattaaag atgtaattaa gatgtttaca caaagagatt ggagtctgtg 480 |
| | | acacttgggg tgcaaaaccc caggaaggga cacaatgggt gaggtgagga tctgtgggag 540 |
| | | gcctggggac agtcacttgg atcccagcta tgagatggca ggccacccag ctgtttctcc 600 |
| | | ttggaaatgt tttggcctgg gggttggggg tggggcatca cactttgata tggagatggg 660 |
| | | gcaacaaagc ctgcaatatc tgggggtgga gaggtcaagt ggatggagtc ttttgagatc 720 |
| | | atgtcaggaa gagggctcga tcccccaaaa tcatggtgac atatggtgtc tcggggttca 780 |
| | | caggagctat gtctaaaata caaaagtaaa 810 |
| 96 | SEQ ID NO: 16 | tctgcagaag cctgccattc caccatttaa acctgtgact ccaggcctta agcctgttga 60 |
| | (Mus musculus) | aggtcgagtc ccagaagggt catatgtgca actgcctagg gagagttccc actcgcaggg 120 |
| | | ccaagaggag tcccccggtc tgaggtgtgg gggcggggac gtgcactggg cgctgggacc 180 |
| | | acggctgggg ctcaggactc gc 202 |
| 97 | SEQ ID NO: 17 | tgcctcagtt tcttcgccta gaaagccggg tctaagggta catgccctga ttcttttctg 60 |
| | (Mus musculus) | gggtgtctcg aattttaaac aacacatact gttctgggct gatgacaaga ggaagtactg 120 |
| | | gtcggtggct gatggacatc caccatggtg gcaactggag ggagggggaa cggacgttga 180 |
| | | aaccctgccc tcctggaatc tgtcgcatgc acgcacgttg acaatgcttg gcactgggga 240 |
| | | caggctggga tggatggagc ggagcgtgag gaggagtggg catgcaggcc cgagtgtctg 300 |
| | | ttttgctgat tgctcctttt gctttcaagg agattaaact atttttagtc catgcctact 360 |
| | | gctggtgaga cgctggagga agcctttcca tcgttgagat tttctggaag ctgccaagtg 420 |
| | | tggtcttcag ctcaattctg ggagcctccc agagtgggag ggaggaacat ttccatctgg 480 |
| | | gggcttcggg gacaggctaa gatcttccct ggggtccttg ctgcgctggc ctcctcaaac 540 |
| | | cacgctgcct cggcctgcat aaagcagtaa tctgatgtgc ccgatgtttg taacgctgtg 600 |
| | | tttaaaaaaa gtaatttatt ttctaattat tccttgtctt gcataaccat gcattgccaa 660 |
| | | agtgtcgcta tttaaaatat ttatctctcc acgccgcagg agcagctctg gagcgtggag 720 |
| | | ggggaagaaa taaaagtccg cgtgccagtc gcaggcatat tactttgact cgtcctggtg 780 |
| | | gctttgacgt ctccctgtaa atacatttat ttttcattag gacgtttctg agcttgtggc 840 |
| | | ccccggagag cggagtgatt acgctgttca tctgcaagcg atgcaataga ggggtactcg 900 |
| | | cagaatgact tccgcccaga gcatcctgcg cctgtct 937 |
| 98 | SEQ ID NO: 18 | taaaatacct tatttttttc cagtctctaa actgctaatc tcccaggcta agggattctg 60 |
| | (Mus musculus) | ggacaaaggc aaggcctgga agtggaaatc tgtaaaatta gcttcagcgg tattagtgtt 120 |
| | | tgcagttgaa gattgaaaaa ctgctttccc agggcctgat tggaggctcc actctcctcc 180 |
| | | aggaagaggc aaggactctg ggctggcact gaggacaaat cctgggaggc tgctatgggg 240 |
| | | cctgggagcc aggctgcctt gtgctagagg cctagagagt gtctgtgtcc caagtcccaa 300 |
| | | gctaccccca gcagctaaca gcttttccag ttctcaggca cagcaggtgc caagatcacg 360 |
| | | ctctggagtc cagctgggcc ccttcctctt cttttttttt tttttttttt aagacctcct 420 |
| | | ggacactgtt cctctccccc cccccgtgac cccccccctc agttctcaaa cacgtgaggg 480 |
| | | ttgggggagg gttccacagc cagagagagg ggccagctct ggtgcctgtg ggtacgcccg 540 |
| | | cccgtatggc ccatcaggcc tcttgtgtgc ttgattgcct ctgattggct gcagctgaat 600 |
| | | tcagcaaaag ctattatttg cccttgatga gccaatcaga tggcctcatt ggccattcag 660 |
| | | agcaggcacc ggaacctgag ggtggggtgg ggggtggggg atggagatgg gactcagtga 720 |
| | | gggggtggga agctctaaaa cagatgcagg acctgagcct gtctgtgtcc accacgacct 780 |
| | | tcacacaggt cacaccccct tcccctgact tgtcacccca aaccagggct tgttgcccaa 840 |
| | | ccccacctca caattccctc actctgtaac acctttccat atacctctgc atgtctaaac 900 |
| | | ccaagacttg ctctatgaaa tc 922 |
| 99 | SEQ ID NO: 19 | agaccctgct tagcacagct cttagcgggt cctttagggg gtctcccagc gggcccagtg 60 |
| | (Mus musculus) | ggaatgagat aaggaaggac acagctgtcc attctcccgt gcctgctaag gaggaaatgg 120 |
| | | ggccgcctta cataattggg gcaatttgtt ccactcttgt cctcctggta tcatggctat 180 |
| | | caccccctcc ttgctcaggg agtccttgat tgagcgagaa gctcaggcct ccctctctcc 240 |
| | | ctcctgctgg gggttgctga acagagggtg taggagccat aggctctgtc actgctgaga 300 |
| | | tctgccagat gtctaggcca ggagaaaatg gaaagggcta agtcacagca tatgtggcca 360 |
| | | ctcaggccta tagccccaaa tctgcctggt aacccattat gtccccagag aatttgcatg 420 |
| | | ggcggacacc ctcatgccgg gtctcagtaa gggaaggggt gggaggcaaa aatatccctc 480 |
| | | cccaccctga atctccaccc cctcccccca gaaactgaca cttggccttg tctaaggatg 540 |
| | | ggttttccca aaatccttct gaaaaaaaca gaatttcaag agtcactccc tccgggtctc 600 |
| | | agcctagaac atatgcagta tcccctgacg tccataggg 639 |
| 100 | SEQ ID NO: 20 | aaactggcac agtaatggcg ggctgacaga caagggagtc tgtagcaccc gctgcctccg 60 |
| | (Mus musculus) | cccacccctt ctccgagcaa ttaaaaggtg tttatgtggg gctggcagtg gcttctgcct 120 |
| | | cccttccatt acgaacatta agagatcttg acccttccac tttccccgct cttgaaagga 180 |
| | | gctgcagaca cgtggagcca attaggcgca cgcgtgggcg ccaagggcct gagcagcttt 240 |
| | | ttctccctga ttgcggcgtt tacagctgat tattctcccc tcacccaaac agtgctgctt 300 |
| | | cctggcaagg tgccacccag aggagccggc tgggggcccc tggggacagg ggaggactgg 360 |
| | | attagtaaat gggcatctat cgaatggctt tcatatgtgt ggctggaagg gagaagggta 420 |
| | | gggccaggaa tggtggcagc aagggcccag gtagcaatga gggttcttct aacccaccat 480 |
| | | ttagggatag cgatcagaaa agggccctcg aggaggtgac ctaaatgtgt gtagaagctg 540 |
| | | acggccacta cacacacaca cacacacaca cacacacata cacaagcatc cttgtccttg 600 |
| | | gagtcggtca gcatgagcaa gagaaagatg ttcccagtgg ccatgagagt ggagccctcc 660 |
| | | tccctactta catccaggtt ggatggccag gagatcctga gatccttcaa gactcc 716 |
| 101 | SEQ ID NO: 21 | aagccacatc ctgggtggaa atatatggct tcaattccca ctcttccgga tgacctctgt 60 |
| | (Mus musculus) | ggggagccct ggcttcacct tggtccagct tcatccctta gcctcgctgc caggaaggca 120 |
| | | gtgaggtcag aggctggtgc tggcgtg 147 |
| 102 | SEQ ID NO: 22 | cctacctggt gcccgccaac atctgggggc catcctggcc agcgccagcg tggtggtgaa 60 |
| | (Mus musculus) | ggcactgtgc gccgtggtac tgtttctcta cctgctttcc ttcgctgtgg acacgggctg 120 |
| | | cctggccgtc accccaggct accttttccc acccaacttc tggatctgga ccctggccac 180 |
| | | ccacgggctc atggaacagc acgtgtggga cgtggccatt agcctggcca cagtggttgt 240 |
| | | ggccgggcga ttactggagc ccctctgggg agccttggag ctgctcatct tcttctc 297 |
| 103 | SEQ ID NO: 23 | aaacggacgg gcctccgctg aaccagtgag gccccagacg tgcgcataaa taacccctgc 60 |
| | (Homo sapiens) | gtgctgcacc acctggggag agggggagga ccacggtaaa t 101 |
| 104 | SEQ ID NO: 24 | ggagcgagcg catagcaaaa gggacgcggg gtccttttct ctgccggtgg cactgggtag 60 |
| | (homo sapiens) | ctgtggccag gtgtggtact ttgatggggc ccagggctgg a 101 |
| 105 | SEQ ID NO: 25 | gctcaaggaa gcgtcgcagg gtcacagatc tgggggaacc ccggggaaaa gcactgaggc 60 |
| | (Homo sapiens) | aaaaccgccg ctcgtctcct acaatatatg ggagggggag g 101 |
| 106 | SEQ ID NO: 26 | ttgagtacgt tctggattac tcataagacc tttttttttt ccttccgggc gcaaaaccgt 60 |
| | (Homo sapiens) | gagctggatt tataatcgcc ctataaagct ccagaggcgg tcaggcacct gcagaggagc 120 |
| | | cccgccgctc cgccgactag ctgcccccgc gagcaacggc ctcgtgattt ccccgccgat 180 |
| | | ccggtccccg cctccccact ctgcccccgc ctaccccgga gccgtgcagc cgcctctccg 240 |
| | | aatctctctc ttctcctggc gctcgcgtgc gagagggaac tagcgagaac gaggaagcag 300 |
| | | ctggaggtga cgccgggcag attacgcctg tcagggccga gccgagcgga tcgctgggcg 360 |
| | | ctgtgcagag gaaaggcggg agtgcccggc tcgctgtcgc agagccgagg tgggtaagct 420 |
| | | agcgaccacc tggacttccc agcgcccaac cgtggctttt cagccaggtc ctctcctccc 480 |
| | | gcggcttctc aaccaacccc atcccagcgc cggccaccca acctcccgaa atgagtgctt 540 |
| | | cctgccc 547 |
| 107 | SEQ ID NO: 27 | cagcagccga aggcgctact aggaacggta acctgttact tttccagggg ccgtagtcga 60 |
| | (Homo sapiens) | cccgctgccc gagttgctgt gcgactgcgc gcgcggggct a 101 |
| 108 | SEQ ID NO: 28 | gagtgcaagg tgactgtggt tcttctctgg ccaagtccga gggagaacgt aaagatatgg 60 |
| | (Homo sapiens) | gcctttttcc ccctctcacc ttgtctcacc aaagtcccta gtccccggag cagttagcct 120 |
| | | ctttctttcc agggaattag ccagacacaa caacgggaac cagacaccga accagacatg 180 |
| | | cccgccccgt gcgccctccc c 201 |
| 109 | SEQ ID NO: 29 | gctcgctgcc tttcctccct cttgtctctc cagagccgga tcttcaaggg gagcctccgt 60 |
| | (Homo sapiens) | gcccccggct gctcagtccc tccggtgtgc aggaccccgg aagtcctccc cgcacagctc 120 |
| | | tcgcttctct ttgcagcctg tttctgcgcc ggaccagtcg aggactctgg acagtagagg 180 |
| | | ccccgggacg accgagctg 199 |
| 110 | SEQ ID NO: 30 | aaacggacgg gcctccgctg aaccagtgag gccccagacg tgcgcataaa taacccctgc 60 |
| | (Homo sapiens) | gtgctgcacc acctggggag agggggagga ccacggtaaa tggagcgagc gcatagcaaa 120 |
| | | agggacgcgg ggtccttttc tctgccggtg gcactgggta gctgtggcca ggtgtggtac 180 |
| | | tttgatgggg cccagggctg gagctcaagg aagcgtcgca gggtcacaga tctgggggaa 240 |
| | | ccccggggaa aagcactgag gcaaaaccgc cgctcgtctc ctacaatata tgggaggggg 300 |
| | | aggttgagta cgttctggat tactcataag accttttttt tttccttccg ggcgcaaaac 360 |
| | | cgtgagctgg atttataatc gccctataaa gctccagagg cggtcaggca cctgcagagg 420 |
| | | agccccgccg ctccgccgac tagctgcccc cgcgagcaac ggcctcgtga tttccccgcc 480 |
| | | gatccggtcc ccgcctcccc actctgcccc cgcctacccc ggagccgtgc agccgcctct 540 |
| | | ccgaatctct ctcttctcct ggcgctcgcg tgcgagaggg aactagcgag aacgaggaag 600 |
| | | cagctggagg tgacgccggg cagattacgc ctgtcagggc cgagccgagc ggatcgctgg 660 |
| | | gcgctgtgca gaggaaaggc gggagtgccc ggctcgctgt cgcagagccg aggtgggtaa 720 |
| | | gctagcgacc acctggactt cccagcgccc aaccgtggct tttcagccag gtcctctcct 780 |
| | | cccgcggctt ctcaaccaac cccatcccag cgccggccac ccaacctccc gaaatgagtg 840 |
| | | cttcctgccc cagcagccga aggcgctact aggaacggta acctgttact tttccagggg 900 |
| | | ccgtagtcga cccgctgccc gagttgctgt gcgactgcgc gcgcggggct agagtgcaag 960 |
| | | gtgactgtgg ttcttctctg gccaagtccg agggagaacg taaagatatg ggcctttttc 1020 |
| | | cccctctcac cttgtctcac caaagtccct agtccccgga gcagttagcc tctttctttc 1080 |
| | | cagggaatta gccagacaca acaacgggaa ccagacaccg aaccagacat gcccgccccg 1140 |
| | | tgcgccctcc ccgctcgctg cctttcctcc ctcttgtctc tccagagccg gatcttcaag 1200 |
| | | gggagcctcc gtgcccccgg ctgctcagtc cctccggtgt gcaggacccc ggaagtcctc 1260 |
| | | cccgcacagc tctcgcttct ctttgcagcc tgtttctgcg ccggaccagt cgaggactct 1320 |
| | | ggacagtaga ggccccggga cgaccgagct g 1351 |
| 111 | SEQ ID NO: 31 | ggaggaagcc atcaactaaa ctacaatgac tgtaagatac aaaattggga atggtaacat 60 |
| | | attttgaagt tctgttgaca taaagaatca tgatattaat gcccatggaa atgaaagggc 120 |
| | | gatcaacact atggtttgaa aagggggaaa ttgtagagca cagatgtgtt cgtgtggcag 180 |
| | | tgtgctgtct ctagcaatac tcagagaaga gagagaacaa tgaaattctg attggcccca 240 |
| | | gtgtgagccc agatgaggtt cagctgccaa ctttctcttt cacatcttat gaaagtcatt 300 |
| | | taagcacaac taactttttt tttttttttt tttttttgag acagagtctt gctctgttgc 360 |
| | | ccaggacaga gtgcagtagt gactcaatct cggctcactg cagcctccac ctcctaggct 420 |
| | | caaacggtcc tcctgcatca gcctcccaag tagctggaat tacaggagtg gcccaccatg 480 |
| | | cccagctaat ttttgtattt ttaatagata cgggggtttc accatatcac ccaggctggt 540 |
| | | ctcgaactcc tggcctcaag tgatccacct gcctcggcct cccaaagtgc tgggattata 600 |
| | | ggcgtcagcc actatgccca acccgaccaa ccttttttaa aataaatatt taaaaaattg 660 |
| | | gtatttcaca tatatactag tatttacatt tatccacaca aaacggacgg gcctccgctg 720 |
| | | aaccagtgag gccccagacg tgcgcataaa taacccctgc gtgctgcacc acctggggag 780 |
| | | agggggagga ccacggtaaa tggagcgagc gcatagcaaa agggacgcgg ggtccttttc 840 |
| | | tctgccggtg gcactgggta gctgtggcca ggtgtggtac tttgatgggg cccagggctg 900 |
| | | gagctcaagg aagcgtcgca gggtcacaga tctgggggaa ccccggggaa aagcactgag 960 |
| | | gcaaaaccgc cgctcgtctc ctacaatata tgggaggggg aggttgagta cgttctggat 1020 |
| | | tactcataag accttttttt tttccttccg ggcgcaaaac cgtgagctgg atttataatc 1080 |
| | | gccctataaa gctccagagg cggtcaggca cctgcagagg agccccgccg ctccgccgac 1140 |
| | | tagctgcccc cgcgagcaac ggcctcgtga tttccccgcc gatccggtcc ccgcctcccc 1200 |
| | | actctgcccc cgcctacccc ggagccgtgc agccgcctct ccgaatctct ctcttctcct 1260 |
| | | ggcgctcgcg tgcgagaggg aactagcgag aacgaggaag cagctggagg tgacgccggg 1320 |
| | | cagattacgc ctgtcagggc cgagccgagc ggatcgctgg gcgctgtgca gaggaaaggc 1380 |
| | | gggagtgccc ggctcgctgt cgcagagccg aggtgggtaa gctagcgacc acctggactt 1440 |
| | | cccagcgccc aaccgtggct tttcagccag gtcctctcct cccgcggctt ctcaaccaac 1500 |
| | | cccatcccag cgccggccac ccaacctccc gaaatgagtg cttcctgccc cagcagccga 1560 |
| | | aggcgctact aggaacggta acctgttact tttccagggg ccgtagtcga cccgctgccc 1620 |
| | | gagttgctgt gcgactgcgc gcgcggggct agagtgcaag gtgactgtgg ttcttctctg 1680 |
| | | gccaagtccg agggagaacg taaagatatg ggcctttttc cccctctcac cttgtctcac 1740 |
| | | caaagtccct agtccccgga gcagttagcc tctttctttc cagggaatta gccagacaca 1800 |
| | | acaacgggaa ccagacaccg aaccagacat gcccgccccg tgcgccctcc ccgctcgctg 1860 |
| | | cctttcctcc ctcttgtctc tccagagccg gatcttcaag gggagcctcc gtgcccccgg 1920 |
| | | ctgctcagtc cctccggtgt gcaggacccc ggaagtcctc cccgcacagc tctcgcttct 1980 |
| | | ctttgcagcc tgtttctgcg ccggaccagt cgaggactct ggacagtaga ggccccggga 2040 |
| | | cgaccgagct g 2051 |
| 112 | SEQ ID NO: 32 | tcaacagggg gacacttggg aaagaaggat ggggacagag ccgagaggac tgttacacat 60 |
| | (synthetic) | tagagaaaca tcagtgactg tgccagcttt ggggtagact gcacaaaagc cctgaggcag 120 |
| | | cacaggcagg atccagtctg ctggtcccag gaagctaacc gtctcagaca gagcacaaag 180 |
| | | caccgagaca tgtgccacaa ggcttgtgta gagaggtcag aggacagcgt acaggtccca 240 |
| | | gagatcaaac tcaacctcac caggcttggc agcaagcctt taccaaccca cccccacccc 300 |
| | | acccaccctg cacgcgcccc tctcccctcc ccatggtctc ccatggctat ctcacttggc 360 |
| | | cctaaaatgt ttaaggatga cactggctgc tgagtggaaa tgagacagca gaagtcaaca 420 |
| | | gtagatttta ggaaagccag agaaaaaggc ttgtgctgtt tttagaaagc caagggacaa 480 |
| | | gctaagatag ggcccaagta atgctagtat ttacatttat ccacacaaaa cggacgggcc 540 |
| | | tccgctgaac cagtgaggcc ccagacgtgc gcataaataa cccctgcgtg ctgcaccacc 600 |
| | | tggggagagg gggaggacca cggtaaatgg agcgagcgca tagcaaaagg gacgcggggt 660 |
| | | ccttttctct gccggtggca ctgggtagct gtggccaggt gtggtacttt gatggggccc 720 |
| | | agggctggag ctcaaggaag cgtcgcaggg tcacagatct gggggaaccc cggggaaaag 780 |
| | | cactgaggca aaaccgccgc tcgtctccta caatatatgg gagggggagg ttgagtacgt 840 |
| | | tctggattac tcataagacc tttttttttt ccttccgggc gcaaaaccgt gagctggatt 900 |
| | | tataatcgcc ctataaagct ccagaggcgg tcaggcacct gcagaggagc cccgccgctc 960 |
| | | cgccgactag ctgcccccgc gagcaacggc ctcgtgattt ccccgccgat ccggtccccg 1020 |
| | | cctccccact ctgcccccgc ctaccccgga gccgtgcagc cgcctctccg aatctctctc 1080 |
| | | ttctcctggc gctcgcgtgc gagagggaac tagcgagaac gaggaagcag ctggaggtga 1140 |
| | | cgccgggcag attacgcctg tcagggccga gccgagcgga tcgctgggcg ctgtgcagag 1200 |
| | | gaaaggcggg agtgcccggc tcgctgtcgc agagccgagg tgggtaagct agcgaccacc 1260 |
| | | tggacttccc agcgcccaac cgtggctttt cagccaggtc ctctcctccc gcggcttctc 1320 |
| | | aaccaacccc atcccagcgc cggccaccca acctcccgaa atgagtgctt cctgccccag 1380 |
| | | cagccgaagg cgctactagg aacggtaacc tgttactttt ccaggggccg tagtcgaccc 1440 |
| | | gctgcccgag ttgctgtgcg actgcgcgcg cggggctaga gtgcaaggtg actgtggttc 1500 |
| | | ttctctggcc aagtccgagg gagaacgtaa agatatgggc ctttttcccc ctctcacctt 1560 |
| | | gtctcaccaa agtccctagt ccccggagca gttagcctct ttctttccag ggaattagcc 1620 |
| | | agacacaaca acgggaacca gacaccgaac cagacatgcc cgccccgtgc gccctccccg 1680 |
| | | ctcgctgcct ttcctccctc ttgtctctcc agagccggat cttcaagggg agcctccgtg 1740 |
| | | cccccggctg ctcagtccct ccggtgtgca ggaccccgga agtcctcccc gcacagctct 1800 |
| | | cgcttctctt tgcagcctgt ttctgcgccg gaccagtcga ggactctgga cagtagaggc 1860 |
| | | cccgggacga ccgagctg 1878 |
| 113 | hGH polyA; | gggtggcatc cctgtgaccc ctccccagtg cctctcctgg ccctggaagt tgccactcca 60 |
| | SEQ ID | gtgcccacca gccttgtcct aataaaatta agttgcatca ttttgtctga ctaggtgtcc 120 |
| | NO: 327 FROM WO | ttctataata ttatggggtg gaggggggtg gtatggagca aggggcaagt tgggaagaca 180 |
| | 2019109051 | acctgtaggg cctgcggggt ctattgggaa ccaagctgga gtgcagtggc acaatcttgg 240 |
| | | ctcactgcaa tctccgcctc ctgggttcaa gcgattctcc tgcctcagcc tcccgagttg 300 |
| | | ttgggattcc aggcatgcat gaccaggctc agctaatttt tgtttttttg gtagagacgg 360 |
| | | ggtttcacca tattggccag gctggtctcc aactcctaat ctcaggtgat ctacccacct 420 |
| | | tggcctccca aattgctggg attacaggcg tgaaccactg ctcccttccc tgtcctt 477 |
| 114 | synthetic | aataaaagat ctttattttc attagatctg tgtgttggtt ttttgtgtgc ggaccgcacg 60 |
| | polyA; SEQ | tg 62 |
| | ID NO: 326 | |
| | FROM | |
| | WO 2019109051 | |
| 115 | Complete | CCTGCAGGCAGCTGCGCGCTCGCTCGCTCACTGAGGCCGCCCGGGCAAAGCCCGGGCGTC |
| | EXT101 | GGGCGACCTTTGGTCGCCCGGCCTCAGTGAGCGAGCGAGCGCGCAGAGAGGGAGTGGCCA |
| | | ACTCCATCACTAGGGGTTCCTGCGGCCGCACGCGTGGCGCGCCggaggaagccatcaact |
| | | aaactacaatgactgtaagatacaaaattgggaatggtaacatattttgaagttctgttg |
| | | acataaagaatcatgatattaatgcccatggaaatgaaagggcgatcaacactatggttt |
| | | gaaaagggggaaattgtagagcacagatgtgttcgtgtggcagtgtgctgtctctagcaa |
| | | tactcagagaagagagagaacaatgaaattctgattggccccagtgtgagcccagatgag |
| | | gttcagctgccaactttctctttcacatcttatgaaagtcatttaagcacaactaacttt |
| | | ttttttttttttttttttttgagacagagtcttgctctgttgcccaggacagagtgcagt |
| | | agtgactcaatctcggctcactgcagcctccacctcctaggctcaaacggtcctcctgca |
| | | tcagcctcccaagtagctggaattacaggagtggcccaccatgcccagctaatttttgta |
| | | tttttaatagatacgggggtttcaccatatcacccaggctggtctcgaactcctggcctc |
| | | aagtgatccacctgcctcggcctcccaaagtgctgggattataggcgtcagccactatgc |
| | | ccaacccgaccaaccttttttaaaataaatatttaaaaaattggtatttcacatatatac |
| | | tagtatttacatttatccacacaaaacggacgggcctccgctgaaccagtgaggccccag |
| | | acgtgcgcataaataacccctgcgtgctgcaccacctggggagagggggaggaccacggt |
| | | aaatggagcgagcgcatagcaaaagggacgcggggtccttttctctgccggtggcactgg |
| | | gtagctgtggccaggtgtggtactttgatggggcccagggctggagctcaaggaagcgtc |
| | | gcagggtcacagatctgggggaaccccggggaaaagcactgaggcaaaaccgccgctcgt |
| | | ctcctacaatatatgggagggggaggttgagtacgttctggattactcataagacctttt |
| | | ttttttccttccgggcgcaaaaccgtgagctggatttataatcgccctataaagctccag |
| | | aggcggtcaggcacctgcagaggagccccgccgctccgccgactagctgcccccgcgagc |
| | | aacggcctcgtgatttccccgccgatccggtccccgcctccccactctgcccccgcctac |
| | | cccggagccgtgcagccgcctctccgaatctctctcttctcctggcgctcgcgtgcgaga |
| | | gggaactagcgagaacgaggaagcagctggaggtgacgccgggcagattacgcctgtcag |
| | | ggccgagccgagcggatcgctgggcgctgtgcagaggaaagggggagtgcccggctcgct |
| | | gtcgcagagccgaggtgggtaagctagcgaccacctggacttcccagcgcccaaccgtgg |
| | | cttttcagccaggtcctctcctcccgcggcttctcaaccaaccccatcccagcgccggcc |
| | | acccaacctcccgaaatgagtgcttcctgccccagcagccgaaggcgctactaggaacgg |
| | | taacctgttacttttccaggggccgtagtcgacccgctgcccgagttgctgtgcgactgc |
| | | gcgcgcggggctagagtgcaaggtgactgtggttcttctctggccaagtccgagggagaa |
| | | cgtaaagatatgggcctttttccccctctcaccttgtctcaccaaagtccctagtccccg |
| | | gagcagttagcctctttctttccagggaattagccagacacaacaacgggaaccagacac |
| | | cgaaccagacatgcccgccccgtgcgccctccccgctcgctgcctttcctccctcttgtc |
| | | tctccagagccggatcttcaaggggagcctccgtgcccccggctgctcagtccctccggt |
| | | gtgcaggaccccggaagtcctccccgcacagctctcgcttctctttgcagcctgtttctg |
| | | cgccggaccagtcgaggactctggacagtagaggccccgggacgaccgagctgGAATTCG |
| | | CCACCATGGCCCCAAAGAAGAAGCGGAAGGTCGGTATCCACGGAGTCCCAGCAGCCCTCG |
| | | AACCAGGTGAAAAACCTTACAAATGTCCTGAATGTGGGAAATCATTCAGTCGCAGCGACA |
| | | ACCTGGTGAGACATCAACGCACCCATACAGGAGAAAAACCTTATAAATGTCCAGAATGTG |
| | | GAAAGTCCTTCTCACGAGAGGATAACTTGCACACTCATCAACGAACACATACTGGTGAAA |
| | | AACCATACAAGTGTCCCGAATGTGGTAAAAGTTTTAGCCGGAGCGATGAACTTGTCCGAC |
| | | ACCAACGAACCCATACAGGCGAGAAGCCTTACAAATGTCCCGAGTGTGGCAAGAGCTTCT |
| | | CACAATCAGGGAATCTGACTGAGCATCAACGAACTCATACCGGGGAAAAACCTTACAAGT |
| | | GTCCAGAGTGTGGGAAGAGCTTTTCCACAAGTGGACATCTGGTACGCCACCAGAGGACAC |
| | | ATACAGGGGAGAAGCCCTACAAATGCCCCGAATGCGGTAAAAGTTTCTCTCAGAATAGTA |
| | | CCCTGACCGAACACCAGCGAACACACACTGGGAAAAAAACGAGTAAAAGGCCGGCGGCCA |
| | | CGAAAAAGGCCGGCCAGGCAAAAAAGAAAAAGGGATCCGACGCGCTGGACGATTTCGATC |
| | | TCGACATGCTGGGTTCTGATGCCCTCGATGACTTTGACCTGGATATGTTGGGAAGCGACG |
| | | CATTGGATGACTTTGATCTGGACATGCTCGGCTCCGATGCTCTGGACGATTTCGATCTCG |
| | | ATATGTTATAAAAAGAGACCGGTTCACTGTGACAGTAAAAGAGACCGGTTCACTGTGAGA |
| | | ATGAAAGAGACCGGTTCACTGTGATCGGAAAAGAGACCGGTTCACTGTGAGCGGCCTTGA |
| | | AACCCAGCAGACAATGTAGCTCAGTAGAAACCCAGCAGACAATGTAGCTGAATGGAAACC |
| | | CAGCAGACAATGTAGCTTCGGAGAAACCCAGCAGACAATGTAGCTAAGCTTGGGTGGCAT |
| | | CCCTGTGACCCCTCCCCAGTGCCTCTCCTGGCCCTGGAAGTTGCCACTCCAGTGCCCACC |
| | | AGCCTTGTCCTAATAAAATTAAGTTGCATCATTTTGTCTGACTAGGTGTCCTTCTATAAT |
| | | ATTATGGGGTGGAGGGGGGTGGTATGGAGCAAGGGGCAAGTTGGGAAGACAACCTGTAGG |
| | | GCCTGCGGGGTCTATTGGGAACCAAGCTGGAGTGCAGTGGCACAATCTTGGCTCACTGCA |
| | | ATCTCCGCCTCCTGGGTTCAAGCGATTCTCCTGCCTCAGCCTCCCGAGTTGTTGGGATTC |
| | | CAGGCATGCATGACCAGGCTCAGCTAATTTTTGTTTTTTTGGTAGAGACGGGGTTTCACC |
| | | ATATTGGCCAGGCTGGTCTCCAACTCCTAATCTCAGGTGATCTACCCACCTTGGCCTCCC |
| | | AAATTGCTGGGATTACAGGCGTGAACCACTGCTCCCTTCCCTGTCCTTCACGTGCGGACC |
| | | GAGCGGCCGCAGGAACCCCTAGTGATGGAGTTGGCCACTCCCTCTCTGCGCGCTCGCTCG |
| | | CTCACTGAGGCCGGGCGACCAAAGGTCGCCCGACGCCCGGGCTTTGCCCGGGGGGCCTCA |
| | | GTGAGCGAGCGAGCGCGCAGCTGCCTGCAGG |

What is claimed is:

1. A composition comprising recombinant adeno-associated virus (AAV) vectors comprising a heterologous nucleic acid, sodium chloride, potassium chloride, magnesium chloride, phosphate buffer, and a non-ionic surfactant, at pH of 7.2-7.4.

2. The composition of claim 1, demonstrating a conductivity of 15.0-17.0 mS/cm.

3. The composition of claim 1 or claim 2, wherein the non-ionic surfactant is present at a concentration of about 0.001% to about 0.02% (w/V).

4. The composition of any one of claims 1-3, wherein the non-ionic surfactant is a poloxamer.

5. The composition of claim 4, wherein the non-ionic surfactant is poloxamer 188.

6. The composition of claim 5, wherein the non-ionic surfactant is poloxamer 188 present at a concentration of from about 0.001% (w/V) to about 0.005% (w/V).

7. The composition of claim 6, wherein the non-ionic surfactant is poloxamer 188 present at a concentration of about 0.005% (w/V).

8. The composition of any one of claims 1-7, comprising about 145 mM to about 150 mM sodium chloride.

9. The composition of any one of claims 1-8, comprising about 1.5 mM to about 4.5 mM potassium chloride.

10. The composition of any one of claims 1-9, comprising about 0.05 mM to about 1 mM magnesium chloride.

11. The composition of any one of claims 1-10, comprising about 148 mM of sodium chloride, about 3 mM potassium chloride, and about 0.8 mM magnesium chloride.

12. The composition of any one of claims 1-11, comprising phosphate buffer in an amount sufficient to provide about 0.5 mM to about 2 mM phosphate.

13. The composition of claim 12, comprising phosphate buffer in an amount sufficient to provide about 1 mM phosphate.

14. The composition of any one of claims 1-13, wherein the phosphate buffer is sodium phosphate.

15. The composition of claim 1, wherein the composition comprises 1 mM phosphate, 148 mM NaCl, 3 mM KCl, 0.8 mM magnesium chloride, and 0.005% Poloxamer 188.

16. The composition of claim 1, wherein the composition comprises 1 mM Phosphate, 148 mM NaCl, 3 mM KCl, 0.8 mM magnesium chloride, and 0.01% Poloxamer 188.

17. The composition of claim 15 or 16, wherein the composition further comprises 0.05% Trehalose.

18. The composition of any one of claims 1-17, comprising about 5×10¹³ vg/mL to about 1.5×10¹⁴ vg/mL AAV vector.

19. The composition of claim 18, comprising about 8×10¹³ vg/mL AAV vector.

20. The composition of any one of claims 1-17, wherein the composition does not comprise calcium chloride.

21. The composition of any one of claims 1-20, wherein the AAV vector comprises a regulatory element active in neural cells.

22. The composition of claim 21, wherein the regulatory element active in neural cells is active in GABAergic neural cells.

23. The composition of claim 19, wherein the AAV vector comprises a sequence of any one of SEQ ID NOs: 81-112.

24. The composition of any one of claims 1-23, wherein the AAV vector comprises a therapeutic transgene.

25. The composition of claim 24, wherein the therapeutic transgene is associated with a neural disease or disorder.

26. The composition of claim 25, wherein the therapeutic transgene is selected from (i): SCN1A, or (ii) a transcription factor which activates SCN1A.

27. The composition of any one of claims 1-26, wherein the AAV vector comprises a sequence of SEQ ID NO: 115.

28. A method of treating a neurological disorder in a subject, the method comprising directly administering to the central nervous system of a subject in need thereof a composition of any one of claims 1-27.

29. The method of claim 28, wherein the method comprises administering the composition to the subject via intracerebroventricular injection.

30. The method of claim 28 or claim 29, wherein the neurological disorder is Dravet syndrome or epilepsy.


SEQUENCE LISTING (115 sequences)

SEQ ID NO: 1 - DNA, 1463 bp, synthetic construct
  source 1..1463: mol_type = other DNA
ccggtcccagagccgcagctgctgcgcccgcgcgctcccggggacattctaaccgccgccaggtcccgccgcctctcgccccgctattaataccggcggcccgggaggggggcgcagcacgcgccgcgcagccatggggaggctgctggccttagtggtcggcgcggcactggtgtcctcagcctgcgggggctgcgtggaggtggactcggagaccgaggccgtgtatgggatgaccttcaaaattctttgcatctcctgcaagcgccgcagcgagaccaacgctgagaccttcaccgagtggaccttccgccagaagggcactgaggagtttgtcaagatcctgcgctatgagaatgaggtgttgcagctggaggaggatgagcgcttcgagggccgcgtggtgtggaatggcagccggggcaccaaagacctgcaggatctgtctatcttcatcaccaatgtcacctacaaccactcgggcgactacgagtgccacgtctaccgcctgctcttcttcgaaaactacgagcacaacaccagcgtcgtcaagaagatccacattgaggtagtggacaaagccaacagagacatggcatccatcgtgtctgagatcatgatgtatgtgctcattgtggtgttgaccatatggctcgtggcagagatgatttactgctacaagaagatcgctgccgccacggagactgctgcacaggagaatgcctcggaatacctggccatcacctctgaaagcaaagagaactgcacgggcgtccaggtggccgaatagccctggccctgggccccgcctcaaggaagagccagccgtaatggggactctccaggcaccgcctgcccccagcgtgggggtggccactcctgggccccagaaagcctcagagtcctgccgacggagccactggggtgggagggggcagggggcttggctcgcacccccactttcgcctcctccagctcctgccccgccggccgcgcaccgccatgcatgatgggtaaagcaatactgccgctgcccccaccctgcttctgctgcctgtttggggaggggggcggtgaggtgggggcagcggccccgcacccctcctccttgctgatttgcacacattggccgcttcagacacgcacttctggggccagcccctccccgcctcctccctgcctggcggcaggggtcgcgatgatgggctggagcagtttggggcagggggttctgggacccactccgactccccctccccggcatcatttcccctcccgcttcctccggctggacctggggtcccccctccctgtaatgcactcctgccccggcccaacctcgccctctctcaccagccttgaactgtggccacctagaaaggggcccattcagcctcgtctctttacagaagtagttttgttcatgaaataaagactcttggacttgaaaaaaaaaaaaaaaa

SEQ ID NO: 2 - DNA, 8133 bp, synthetic construct
  source 1..8133: mol_type = other DNA
aatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttatttta

SEQ ID NO: 3 - DNA, 4922 bp, synthetic construct
  source 1..4922: mol_type = other DNA
ctctttttagcagcaacatacaagccggccatattagagagatggaaataaagcttccttaatgttgtatatgtctttgaagtacatccgtgcatttttttttagcatccaaccattcctcccttgtagttctcgccccctcaaatcaccctctcccgtagcccacccgactaacatctcagtctctgaaaatgcacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaagaccgcgtggagttctcagggaaccccagcaagtacgatgtgtcggtgatgctgagaaacgtgcagccggaggatgaggggatttacaactgctacatcatgaacccccctgaccgccaccgtggccatggcaagatccatctgcaggtcctcatggaagagccccctgagcgggactccacggtggccgtgattgtgggtgcctccgtcgggggcttcctggctgtggtcatcttggtgctgatggtggtcaagtgtgtgaggagaaaaaaagagcagaagctgagcacagatgacctgaagaccgaggaggagggcaagacggacggtgaaggcaacccggatgatggcgccaagtagtgggtggccggccctgcagcctcccgtgtcccgtctcctcccctctccgccctgtacagtgaccctgcctgctcgctcttggtgtgcttcccgtgacctaggaccccagggcccacctggggcctcctgaacccccgacttcgtatctcccaccctgcaccaagagtgacccactctcttccatccgagaaacctgccatgctctgggacgtgtgggccctggggagaggagagaaagggctcccacctgccagtccctggggggaggcaggaggcacatgtgagggtccccagagagaagggagtgggtgggcaggggtagaggagggggccgctgtccacctgcccagtgcttggcctggcagtggcttcagagaggacctggtggggagggagggctttcctgtgctgacagcgctccctcaggagggccttggcctggcacggctgtgctcctcccctgctcccagcccagagcagccatcaggctggaggtgacgatgagttcctgaaacttggaggggcatgttaaagggatgactgtgcattccagggcactgacggaaagccagggctgcaggcaaagctggacatgtgccctggcccaggaggccatgttgggccctcgtttccattgctagtggcctccttggggctcccgttggctcctaatcccttaggactgtggatgaggccagactggaagagcagctccaggtagggggccatgtttcccagcggggacccaccaacagaggccagtttcaaagtcagctgaggggctgaggggtggggctccatggtgaatgcaggttgctgcaggctctgccttctccatggggtaaccaccctcgcctgggcaggggcagccaaggctgggaaatgaggaggccatgcacagggtggggcagctttctttggggcttcagtgagaactctcccagttgcccttggtggggtttccacctggcttttggctacagagagggaagggaaagcctgaggccggcataaggggaggccttggaacctgagctgccaatgccagccctgtcccatctgcagccacactactcgctcctctcccaacaactcccttcgtggggacaaaagtgacaattgtaggccaggcacagtggctcacgcctgtaatcccagcactttgggaggccaaggcgggtggattacctccatctgtttagtagaaatgggcaaaaccccatctctactaaaaatacaagaattagctgggcgtggtggcgtgtgcctgtaatcccagctatttgggaggctgaggcaggagaatcgcttgagcccgggaagcagaggttgcagtgaactgagatagtgatagtgccactgcaattcagcctgggtgacatagagagactccatctcaaaaaaaaaaaaaaaaaaaaaaaaaggcaattgtgcggagccaaccatcactgccattacactcacattcaatggaaattgattaggcctgagagaaggagcaagaggcagaaacactgactctgagaccatgaccttggaagatcctcagagatcaactagccaagtcactcacttaaaatggggaaactgaggttccaactgacagaaactgagggtcctgccctcttctctggctcctcagtctccagtttgaagcaacagagccatcattttcccagtgggaggggcctctgtgtcctggtctatgatggggcgagttggaggggtgggcttctctagccctgccccctcccaggagcccagcccctctggccaggactgaggaggagtggagggaagcagggagggagggagcaagtgtgaagggcacagctgctggcaaatgccccattccctgccccagcacttcccagcctttccaggagctctgccccacccctactcaaggagtcaggtgcccagggtagggaataagtctaggaaagggggtggagacaggaccgtgacagtctcagggaccaatccctgcctctgcccatcagattgccaatttggggagacagcaatatcatctgggagattcagcttaatttgcttcaattcagcaaccatttattgagcatctattatgtgccaggcactgtgccgggcactggggatatacacaaagccataagcactggggactgccaggttggcactgtagggaccaagattaactttttggtgcatctaattccacctctgcaagacactgcttccaaggctacagttggggaagatgaagagattctagaatctgtgccctgggaagggagaaacagcagggaagaggggcaatacaacagggtcaagggtgcagaggccaggcagagtgggtgcagagtgggggatgggcaaccagcctggtaacacctgggtgaggtgagcgagtcaagcccatctggaaatgctaaactcatcagctcagtacctaactgccaatcaacacactgccaatgcgggtgggctaagccgccaggatgcctgcagcagatggacgctgcccatccttgagccagccagctgggggcagagaggtgggtggcgggcacctcatgccagctgagaccaagagtggttgtatacatttctgcagcttgcagatttcatttttttggtcaccctttaagtgtccttctctcccaggatgagtgaggagctgtgctgatgttgggaacttatctttttccaggatccataacatttaggggaattatttgcgggctttcctagtgtattctaagaagttctaactctccatcaagaagcccaagattcctggagtctttttttcctctgccctgatcaactggggcccaggaaaagcaatgtctcttcacgggcctcagggccccagcagatggggctgggtcaggaaccatctctgtcccaggtgctcagccctcatatgcattttcttctggagcctcttagaatctcctccagaggactcatagagctgcctcccccaggatggtgaagttgggagcaaaatggggcttctgtaagtagaggtggaaaaccagagagatgtttccacagcagccatttccctaccctgtttcctaacttatgctgacaagttcttcctcttcctaccgtggggttcgagccccacttctggttgcatgttcccaccaccacgctctgctggttttagtcggctgactttctcaactgccccagcctctcctgctctctcagaacctccctctgcccccaccccccatctgtctcttcctcctaattaagggccactgcaaagagagacgggctggctaacccatgggatttagcttataagaacaaagcacaattattcaggactgaactgctttgaggtgccttggaactggctttggaaatagctatctgccccctagctatggactcttcacagttatttccagtttataaaaaggaaaagctgatcggatccatgtttctctctgcgagtctccttggggaggttgggctggtaaatatttgcaaggtcaggttaagcagacagacaattgaaaatactgggcaggtccaaaaggatagttccaggccataggaatggcttggtgcagttaacgggtgatgaagcaagaaatcttccttcctacttatgtcttaaagatggacagcctgtgtctcttgctgtgcctcttgctggcgtcttcctttattgaagcttgctgatgcactgtacatagtctctctctctaggtatagatatattatatatacaaatatcaaacatctcaccacacccaccccaaaagttacactgtgtgtgtgtggggaggtgtaataacttctcagtgttttgagttgattcaccaaaggcaaattttggaatgttcttgcgctttgtgcacttggaggggaaaacccaactacatatgcactttcttggttgtgtgtgctatagagaggttggaaggggtggttggaaggggagagaaactgagatggccctctgtgactcgactgctgggatgtatctgcttttgggagcagactgagtttcttttgcaatttgtcttattgtttgggtctactggcaacaataaactatctcccctgaaaatc

SEQ ID NO: 4 - DNA, 3134 bp, synthetic construct
  source 1..3134: mol_type = other DNA
ggcggccgctcccatgggtgtcgctgggccgcgccatgcctaagggggcgccgcgatgggccaaggggacgagagcgagcgcatcgtgatcaacgtgggcggcacgcgccaccagacgtaccgctcgaccctgcgcacgctgcccggcacgcggctcgcctggctggcggagcccgacgcccacagccacttcgactatgacccgcgtgctgacgagttcttcttcgaccgccaccccggcgtcttcgcgcacatcctgaactactaccgcacgggcaagctgcactgcccagccgacgtgtgcgggccgctctacgaggaggagctggccttctggggcatcgacgagaccgacgtggagccctgctgctggatgacgtaccgccagcaccgcgacgccgaggaggctctggacagcttcggcggcgctcctctggacaacagcgccgacgacgcggacgccgacggccctggcgactcgggcgacggcgaggacgagctggagatgaccaagcgcctggcgctcagtgactccccggatggccggcctggcggcttttggcgccgctggcagccgcgcatctgggcgctcttcgaggacccgtactcgtcccgctacgcgcggtatgtggccttcgcttccctcttcttcatcctggtctccatcaccaccttctgcctggagacccacgagcgcttcaaccccatcgtgaacaagacggagatcgagaacgttcgcaatggcacgcaagtgcgctactaccgggaggccgagacggaggccttccttacctacatcgagggcgtctgtgtggtctggttcaccttcgagttcctcatgcgtgtcatcttctgccccaacaaggtagagttcatcaagaactcgctcaacatcattgactttgtggccatcctgcccttctacctggaggtggggctgagcggcctgtcctccaaggcagccaaggacgtgctgggcttcctgcgcgtcgtccgcttcgtgcgcatcttgcgcatctttaagctgacccgccactttgtgggcctgcgggtcctgggccacacgctccgagccagcaccaacgagttcctgctgctcatcatcttcctggccttgggcgtgctgatcttcgccaccatgatctactacgccgagaggataggggcacagcccaatgaccccagcgccagtgagcacacgcactttaagaacatccccatcggcttctggtgggccgtggtcaccatgacgaccctgggctatggagacatgtacccgcagacgtggtccggcatgctggtgggggctctgtgtgcgctggcgggcgtgctcaccatcgccatgcccgtgcccgtcatcgtgaacaatttcgggatgtattactccttagccatggctaagcagaaactaccaaagaaaaaaaagaagcatattccgcggccaccgcagctgggatctcccaattattgtaaatctgtcgtaaactctccacaccacagtactcagagtgacacatgtccgctggcccaggaagaaattttagaaattaacagagcagattccaaactgaatggggaggtggcgaaggccgcgctggcgaacgaagactgcccccacatagaccaggccctcactcccgatgagggcctgccctttacgcgctcgggcacccgcgagagatacggaccctgcttcctcttatcaaccggggagtacgcgtgcccacctggtggaggaatgagaaaggatctttgcaaagaaagccctgtcattgctaagtatatgccgacagaggctgtgagagtgacttgaccaggcggcttggccgaggacactggtggctattaagcatctgggtggacctgcagcccctcctcaccctcggacagagtaaattcacgccatgcaggtttgccggacgagtccgagtggcccaggcattgtactaggacggacgtagctttttctacggccaaatggtaactgaccgtagaggatttcttttccttcttttcattttttaaaattttattttatttggggagggggggtggaggggctccttagcatgacttgcatgaagttaaacagaaaacccagcaaaccaaacccaccaaccccctgcaactgtatgattaccctgaacaacaataatgaaaagaaaaacatcaaagccctctattttctttcaaagctcttgactttcacacacgttgttggagccaaactgtaacggcgttcagtagaaacctgtacatttctgggggtgaggggcgaggggaggagggacagagatggggaatggattgcttcctttttgtacacaagatcaagaggaaagcttccaaaaagggcagtcggtcagtcatcggtcatattgacctcaccttcatagttcatctctcacgtggaaactgagaactttgctccaaatagaattgtggaaactcgcacggctgccttcttccccgtttggcatgcttgtgacccagcggatgtctccccaagacccctgacagcagctagtctaggttgattctctcatcttatcggtgtgtagatccagtgtgaccaactttcataacaaattgttcatagtaaataatcaccaccgtatggattttccaagtgttccattaaaaccaggatgtagagcaccaaaagatcaggaccacgggaggagcagcctcctccacctttactaaggcacaacctggcattgtatgcaatttagtacttcagagtaaaaacctgcatgcccaatgttatgcaaagcgattctagcatgaaataaattttgtatcatggtttctgtatagtctaaagcagatttgttttcctgaagcagtcggaggtttgcagagacacacttctgcatctcgaataaatatagtattttcccaacagaaacagaggacagtagcttggctttggtctgattctaaaattagtggggtgggggggaaggatgatatttttgaaaaacacatgcttgagttgaaaaaaatgcaacatctcaagctttcactgcagctcacaacatttcctggaaagagagcaatgatgctttagctcaacaacctcccctccctgggccctggcccccaataataaaaaactgacttttgagatg

SEQ ID NO: 5 - DNA, 3176 bp, synthetic construct
  source 1..3176: mol_type = other DNA
accggtcccgcctcccttgacgccccgccccgcccctcctccctgctctcttagaaagagccaatcgctccgtctcattggcggccccgactcgtccagctgggtcgcgttagagtgcccgccccactcctcccagccacagccccgccccacctcccccaggccaatcagtttggttcctctcccctaagccacgcccccgctacctcgctcctcctcccacccaatcccgtcggtctctccccccgtgtccccgcccctgcgtccccgcccctcccgccccgccccccgtccaatgctgagctcagtctgcgtctcgtccttccgcgggcgccagggggccagcaagcagcagccggcgccaccgccgcagccgcccgagtccccgccgccgccaccgctgccgccgcagcagcagcagcctgcgcagcccggccccgccgcgtccccggcgggccccccggcaccccgcgggcccggggaccggcgcgccgagccatgccccgggctgccggcggcggccatggggcggcacggcggcggcggtggcgacagcggcaagatcgtgatcaacgtgggcggcgtgcgccatgagacgtaccgctcgacgctgcgcaccctgccggggacgcggctggccggcctgacggagcccgaggcggcggcacgcttcgactacgacccgggcgccgacgagttcttctttgaccggcacccgggagtcttcgcgtacgtgctcaactactaccgcaccggcaagctgcactgcccagccgacgtgtgcgggcccctgtttgaggaggagctcggcttctggggcatcgacgagaccgacgtggaggcctgctgctggatgacctaccggcagcatcgcgacgctgaggaggcgctcgactccttcgaggcgcccgaccccgcgggcgccgccaacgccgccaacgccgcaggcgcccacgacggaggcctggacgacgaggcgggcgcgggcggcggcggcctggacggagcgggcggcgagctcaagcgcctctgcttccaggacgcgggcggcggcgccggggggccgccagggggcgcgggcggcgcgggcggcacatggtggcgccgctggcagccccgcgtgtgggcgctcttcgaggacccctactcgtcgcgggctgccaggtatgtggccttcgcctccctcttcttcatcctcatctccatcaccaccttctgcctggaaacccatgagggcttcatccatattagcaacaagacggtgacccaggcctccccgatccccggggcacctccggagaacatcaccaacgtggaggtggagacggagcccttcctgacctacgtggagggggtgtgcgtggtctggttcaccttcgagttcctcatgcgcatcaccttctgcccagacaaggtggagtttcttaaaagcagcctcaacatcatcgactgtgtggccatcctgcccttctatctcgaggtgggcctctcgggcctcagctccaaggccgccaaagacgtgctgggcttcctgcgggtggtccgcttcgtccgcatcctgcgcatcttcaagctgacccggcacttcgtggggctgcgcgtgctgggacacacgctccgcgccagcaccaacgagttcctgctgctcatcatcttcctggccctgggggtgctcatcttcgccaccatgatttactacgctgagcgcattggcgccgaccccgatgacatcctgggctccaaccacacctacttcaagaacatccccattggcttctggtgggctgtggtcaccatgacgaccctgggctatggagacatgtaccccaagacgtggtcggggatgctggtcggggcgctgtgtgccctggcgggggtgctgaccatcgccatgcctgtgcccgtcattgtcaacaactttggcatgtactattcgctggccatggccaagcagaagctgcccaagaagaagaacaaacacatcccccggcccccgcaaccgggctcgcccaactactgcaagcctgacccacccccgccacccccgccccacccgcaccacggcagcgggggcatcagcccgccgccacccatcaccccaccctccatgggggtgactgtggccggggcctacccagcggggccccacacgcaccccgggctgctcagggggggagcgggtgggctggggatcatggggctgcctcctctgccagcccccggcgagccttgcccgttggctcaggaggaggtgattgagatcaaccgggcagatcctcgccccaatggggatccggcagcagctgcgcttgcccacgaggactgcccagccattgaccagcctgccatgtccccggaagacaagagccccatcacgcctggaagccgtggccgctatagccgggaccgagcctgcttcctcctcaccgactatgccccttcccctgatggctccatccgaaaagccactggtgctcccccactgcccccccaagactggcgtaagccaggccccccaagcttcttgcccgacctcaacgccaacgccgcggcctggatatccccctagtggacgaaccccctccccccgggctcttgtcaccgcctgagacctcgcgagactttcggtcccccccgccccttccccccaggttagcaattgggaatggctgggagggggtgtccccaagacactgggcttcaaatctccccccaagccctccctcctacgatcaagacaccttttgtccagacagctcccttagcgttgcctggaaaagccacagagctccctgcgggggtgcagaaccctgcaggcagccccaggctttgcagaaaccacacacactgttgctggatcttgagtcccattagctgtgagatgcctgtgagagccttcccgcacccgtcagaagctcagagccttcttggaccctttaaagatgtccctctccccagccctccgcctcaccgccccccacccccgaccccgtccttagaatcttctggaggagcctccctccccccccacccccccccactgctcctggagttctcatcggattccccaagggcactgccagcttcgctccactcagcccccttgcagaccccaccccctgcctgctctctttccctacaactaggtcagcccccagccccgctacggcggccgc

SEQ ID NO: 6 - protein, 206 aa, synthetic construct
DFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 7 - protein, 1973 aa, synthetic construct
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGAFEDIYIDQRKTIKTMLEYADKVFTYIFIMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVINFSVATEESAEPLSEDLEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTS

SEQ ID NO: 8 - protein, 176 aa, synthetic construct
LEPGEKPYKCPECGKSFSTKNSLTEHQRTHTGEKPYKCPECGKSFSRADNLTEHQRTHTGEKPYKCPECGKSFSQLAHLRAHQRTHTGEKPYKCPECGKSFSTKNSLTEHQRTHTGEKPYKCPECGKSFSQAGHLASHQRTHTGEKPYKCPECGKSFSTHLDLIRHQRTHTGKKTS

SEQ ID NO: 9 - protein, 176 aa, synthetic construct
LEPGEKPYKCPECGKSFSQAGHLASHQRTHTGEKPYKCPECGKSFSREDNLHTHQRTHTGEKPYKCPECGKSFSTSGNLTEHQRTHTGEKPYKCPECGKSFSTHLDLIRHQRTHTGEKPYKCPECGKSFSQKSSLIAHQRTHTGEKPYKCPECGKSFSQAGHLASHQRTHTGKKTS

SEQ ID NO: 10 - protein, 176 aa, synthetic construct
LEPGEKPYKCPECGKSFSTTGNLTVHQRTHTGEKPYKCPECGKSFSTSGELVRHQRTHTGEKPYKCPECGKSFSREDNLHTHQRTHTGEKPYKCPECGKSFSTSGNLTEHQRTHTGEKPYKCPECGKSFSQSSSLVRHQRTHTGEKPYKCPECGKSFSQRANLRAHQRTHTGKKTS

SEQ ID NO: 11 - protein, 176 aa, synthetic construct
LEPGEKPYKCPECGKSFSSRRTCRAHQRTHTGEKPYKCPECGKSFSTTGALTEHQRTHTGEKPYKCPECGKSFSRSDELVRHQRTHTGEKPYKCPECGKSFSRNDALTEHQRTHTGEKPYKCPECGKSFSQSGDLRRHQRTHTGEKPYKCPECGKSFSTSHSLTEHQRTHTGKKTS

SEQ ID NO: 12 - protein, 176 aa, synthetic construct
LEPGEKPYKCPECGKSFSRKDNLKNHQRTHTGEKPYKCPECGKSFSDPGALVRHQRTHTGEKPYKCPECGKSFSREDNLHTHQRTHTGEKPYKCPECGKSFSDPGALVRHQRTHTGEKPYKCPECGKSFSTSGELVRHQRTHTGEKPYKCPECGKSFSRKDNLKNHQRTHTGKKTS

SEQ ID NO: 13 - protein, 232 aa, synthetic construct
LEPGEKPYKCPECGKSFSSKKALTEHQRTHTGEKPYKCPECGKSFSSPADLTRHQRTHTGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTS

SEQ ID NO: 14 - protein, 176 aa, synthetic construct
LEPGEKPYKCPECGKSFSSPADLTRHQRTHTGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGKKTS

SEQ ID NO: 15 - protein, 176 aa, synthetic construct
LEPGEKPYKCPECGKSFSSKKALTEHQRTHTGEKPYKCPECGKSFSSPADLTRHQRTHTGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGKKTS

SEQ ID NO: 16 - protein, 232 aa, synthetic construct
LEPGEKPYKCPECGKSFSDCRDLARHQRTHTGEKPYKCPECGKSFSRNDALTEHQRTHTGEKPYKCPECGKSFSRNDALTEHQRTHTGEKPYKCPECGKSFSSPADLTRHQRTHTGEKPYKCPECGKSFSDPGNLVRHQRTHTGEKPYKCPECGKSFSQRAHLERHQRTHTGEKPYKCPECGKSFSQSSSLVRHQRTHTGEKPYKCPECGKSFSHRTTLTNHQRTHTGKKTS

SEQ ID NO: 17 - protein, 176 aa, synthetic construct
LEPGEKPYKCPECGKSFSRNDALTEHQRTHTGEKPYKCPECGKSFSSPADLTRHQRTHTGEKPYKCPECGKSFSDPGNLVRHQRTHTGEKPYKCPECGKSFSQRAHLERHQRTHTGEKPYKCPECGKSFSQSSSLVRHQRTHTGEKPYKCPECGKSFSHRTTLTNHQRTHTGKKTS

SEQ ID NO: 18 - protein, 204 aa, synthetic construct
LEPGEKPYKCPECGKSFSRNDALTEHQRTHTGEKPYKCPECGKSFSDPGHLVRHQRTHTGEKPYKCPECGKSFSTSGELVRHQRTHTGEKPYKCPECGKSFSTHLDLIRHQRTHTGEKPYKCPECGKSFSSKKALTEHQRTHTGEKPYKCPECGKSFSQLAHLRAHQRTHTGEKPYKCPECGKSFSRSDHLTNHQRTHTGKKTS

SEQ ID NO: 19 - protein, 176 aa, synthetic construct
LEPGEKPYKCPECGKSFSRSDNLVRHQRTHTGEKPYKCPECGKSFSHRTTLTNHQRTHTGEKPYKCPECGKSFSREDNLHTHQRTHTGEKPYKCPECGKSFSTSHSLTEHQRTHTGEKPYKCPECGKSFSQSSSLVRHQRTHTGEKPYKCPECGKSFSREDNLHTHQRTHTGKKTS

SEQ ID NO: 20 - protein, 176 aa, synthetic construct
LEPGEKPYKCPECGKSFSDPGALVRHQRTHTGEKPYKCPECGKSFSRSDNLVRHQRTHTGEKPYKCPECGKSFSQSGDLRRHQRTHTGEKPYKCPECGKSFSTHLDLIRHQRTHTGEKPYKCPECGKSFSTSGNLVRHQRTHTGEKPYKCPECGKSFSRSDNLVRHQRTHTGKKTS

SEQ ID NO: 21 - protein, 260 aa, synthetic construct
LEPGEKPYKCPECGKSFSRRDELNVHQRTHTGEKPYKCPECGKSFSRSDHLTNHQRTHTGEKPYKCPECGKSFSRSDDLVRHQRTHTGEKPYKCPECGKSFSRSDNLVRHQRTHTGEKPYKCPECGKSFSHRTTLTNHQRTHTGEKPYKCPECGKSFSREDNLHTHQRTHTGEKPYKCPECGKSFSTSHSLTEHQRTHTGEKPYKCPECGKSFSQSSSLVRHQRTHTGEKPYKCPECGKSFSREDNLHTHQRTHTGKKTS

SEQ ID NO: 22 - protein, 177 aa, synthetic construct
RPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 23 - protein, 177 aa, synthetic construct
RPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDK

SEQ ID NO: 24 - protein, 264 aa, synthetic construct
RPYACPVESCDRRFSRRDELNVHIRIHTGQKPFQCRICMRNFSRSDHLTNHIRTHTGEKPFACDICGRKFARSDDLVRHTKIHLRQKDRPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKD

SEQ ID NO: 25 - protein, 264 aa, synthetic construct
RPYACPVESCDRRFSDPGALVRHIRIHTGQKPFQCRICMRNFSRSDNLVRHIRTHTGEKPFACDICGRKFAQSGDLRRHTKIHLRQKDRPYACPVESCDRRFSTHLDLIRHIRIHTGQKPFQCRICMRNFSTSGNLVRHIRTHTGEKPFACDICGRKFARSDNLVRHTKIHLRQKDRPYACPVESCDRRFSQSGHLTEHIRIHTGQKPFQCRICMRNFSERSHLREHIRTHTGEKPFACDICGRKFAQAGHLASHTKIHLRQKD

SEQ ID NO: 26 - protein, 175 aa, synthetic construct
RPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEK

SEQ ID NO: 27 - protein, 175 aa, synthetic construct
RPHACPAEGCDRRFSRSDNLVRHLRIHTGHKPFQCRICMRSFSHRTTLTNHIRTHTGEKPFACEFCGRKFAREDNLHTHAKIHLKQKEHACPAEGCDRRFSTSHSLTEHLRIHTGHKPFQCRICMRSFSQSSSLVRHIRTHTGEKPFACEFCGRKFAREDNLHTHAKIHLKQKEK

SEQ ID NO: 28 - protein, 261 aa, synthetic construct
RPHACPAEGCDRRFSRRDELNVHLRIHTGHKPFQCRICMRSFSRSDHLTNHIRTHTGEKPFACEFCGRKFARSDDLVRHAKIHLKQKEHACPAEGCDRRFSRSDNLVRHLRIHTGHKPFQCRICMRSFSHRTTLTNHIRTHTGEKPFACEFCGRKFAREDNLHTHAKIHLKQKEHACPAEGCDRRFSTSHSLTEHLRIHTGHKPFQCRICMRSFSQSSSLVRHIRTHTGEKPFACEFCGRKFAREDNLHTHAKIHLKQKEK

SEQ ID NO: 29 - protein, 50 aa, synthetic construct
DALDDFDLDMLGSDALDDFDLDMLGSDALDDFDLDMLGSDALDDFDLDML

SEQ ID NO: 30 - protein, 523 aa, synthetic construct
DALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 31 - protein, 275 aa, synthetic construct
MSGLEMADHMMAMNHGRFPDGTNGLHHHPAHRMGMGQFPSPHHHQQQQPQHAFNALMGEHIHYGAGNMNATSGVRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSC

SEQ ID NO: 32 - protein, 274 aa, synthetic construct
SGLEMADHMMAMNHGRFPDGTNGLHHHPAHRMGMGQFPSPHHHQQQQPQHAFNALMGEHIHYGAGNMNATSGVRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSC

SEQ ID NO: 33 - protein, 184 aa, synthetic construct
MADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 34 - protein, 184 aa, synthetic construct
AADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 35 - protein, 543 aa, synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDELTRHIRIHTGQKPFQCRICMRNFSRSDHLTTHIRTHTGEKPFACDICGRKFARSDERKRHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 36 - protein, 387 aa, synthetic construct
MTGKLAEKLPVTMSSLLNQLPDNLYPEEIPSALNLFSGSSDSVVHYNQMATENVMDIGLTNEKPNPELSYSGSFQPAPGNKTVTYLGKFAFDSPSNWCQDNIISLMSAGILGVPPASGALSTQTSTASMVQPPQGDVEAMYPALPPYSNCGDLYSEPVSFHDPQGNPGLAYSPQDYQSAKPALDSNLFPMIPDYNLYHHPNDMGSIPEHKPFQGMDPIRVNPPPITPLETIKAFKDKQIHPGFGSLPQPPLTLKPIRPRKYPNRPSKTPLHERPHACPAEGCDRRFSRSDELTRHLRIHTGHKPFQCRICMRSFSRSDHLTTHIRTHTGEKPFACEFCGRKFARSDERKRHAKIHLKQKEKKAEKGGAPSASSAPPVSLAPVVTTCA

SEQ ID NO: 37 - protein, 8 aa, synthetic construct
GGSGGGSG

SEQ ID NO: 38 - protein, 16 aa, synthetic construct
GGSGGGSGGGSGGGSG

SEQ ID NO: 39 - protein, 52 aa, synthetic construct
  SITE 19: note = Recognition Helix
  SITE 41: note = Recognition Helix
LEPGEKPYKCPECGKSFSXHQRTHTGEKPYKCPECGKSFSXHQRTHTGKKTS

SEQ ID NO: 40 - protein, 753 aa, synthetic construct
MAPKKKRKVGIHGVPAALEPGEKPYKCPECGKSFSRSDNLVRHQRTHTGEKPYKCPECGKSFSHRTTLTNHQRTHTGEKPYKCPECGKSFSREDNLHTHQRTHTGEKPYKCPECGKSFSTSHSLTEHQRTHTGEKPYKCPECGKSFSQSSSLVRHQRTHTGEKPYKCPECGKSFSREDNLHTHQRTHTGKKTSKRPAATKKAGQAKKKKGSYPYDVPDYALEEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 41 - protein, 753 aa, synthetic construct
MAPKKKRKVGIHGVPAALEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTSKRPAATKKAGQAKKKKGSYPYDVPDYALEEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 42 - protein, 272 aa, synthetic construct
MAPKKKRKVGIHGVPAALEPGEKPYKCPECGKSFSRSDNLVRHQRTHTGEKPYKCPECGKSFSHRTTLTNHQRTHTGEKPYKCPECGKSFSREDNLHTHQRTHTGEKPYKCPECGKSFSTSHSLTEHQRTHTGEKPYKCPECGKSFSQSSSLVRHQRTHTGEKPYKCPECGKSFSREDNLHTHQRTHTGKKTSKRPAATKKAGQAKKKKGSYPYDVPDYALEDALDDFDLDMLGSDALDDFDLDMLGSDALDDFDLDMLGSDALDDFDLDML

SEQ ID NO: 43 - protein, 272 aa, synthetic construct
MAPKKKRKVGIHGVPAALEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTSKRPAATKKAGQAKKKKGSYPYDVPDYALEDALDDFDLDMLGSDALDDFDLDMLGSDALDDFDLDMLGSDALDDFDLDML

SEQ ID NO: 44 - protein, 580 aa, synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 45 - protein, 394 aa, synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDK

SEQ ID NO: 46 - protein, 580 aa, synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 47 - protein, 394 aa, synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 48 - protein, 388 aa, synthetic construct
MQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 49 - protein, 479 aa, synthetic construct
MQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDKLEMSGLEMADHMMAMNHGRFPDGTNGLHHHPAHRMGMGQFPSPHHHQQQQPQHAFNALMGEHIHYGAGNMNATSGIRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSC

SEQ ID NO: 50 - protein, 476 aa, synthetic construct
MSGLEMADHMMAMNHGRFPDGTNGLHHHPAHRMGMGQFPSPHHHQQQQPQHAFNALMGEHIHYGAGNMNATSGVRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSCQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 51 - protein, 554 aa, synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGRPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 52 - protein, 362 aa, synthetic construct
MRPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKLEMADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSC

SEQ ID NO: 53 - protein, 479 aa, synthetic construct
MRPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKKAEKGGAPSASSAPPVSLAPVVTTCALEMSGLEMADHMMAMNHGRFPDGTNGLHHHPAHRMGMGQFPSPHHHQQQQPQHAFNALMGEHIHYGAGNMNATSGIRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSC

SEQ ID NO: 54 - protein, 476 aa, synthetic construct
MSGLEMADHMMAMNHGRFPDGTNGLHHHPAHRMGMGQFPSPHHHQQQQPQHAFNALMGEHIHYGAGNMNATSGVRHAMGPGTVNGGHPPSALAPAARFNNSQFMGPPVASQGGSLPASMQLQKLNNQYFNHHPYPHNHYMPDLHPAAGHQMNGTNQHFRDCNPKHSGGSSTPGGSGGSSTPGGSGSSSGGGAGSSNSGGGSGSGNMPASVAHVPAAMLPPNVIDTDFIDEEVLMSLVIEMGLDRIKELPELWLGQNEFDFMTDFVCKQQPSRVSCRPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKKAEKGGAPSASSAPPVSLAPVVTTCA

SEQ ID NO: 55 - protein, 559 aa, synthetic construct
MTGKLAEKLPVTMSSLLNQLPDNLYPEEIPSALNLFSGSSDSVVHYNQMATENVMDIGLTNEKPNPELSYSGSFQPAPGNKTVTYLGKFAFDSPSNWCQDNIISLMSAGILGVPPASGALSTQTSTASMVQPPQGDVEAMYPALPPYSNCGDLYSEPVSFHDPQGNPGLAYSPQDYQSAKPALDSNLFPMIPDYNLYHHPNDMGSIPEHKPFQGMDPIRVNPPPITPLETIKAFKDKQIHPGFGSLPQPPLTLKPIRPRKYPNRPSKTPLHERPHACPAEGCDRRFSRRDELNVHLRIHTGHKPFQCRICMRSFSRSDHLTNHIRTHTGEKPFACEFCGRKFARSDDLVRHAKIHLKQKEHACPAEGCDRRFSRSDNLVRHLRIHTGHKPFQCRICMRSFSHRTTLTNHIRTHTGEKPFACEFCGRKFAREDNLHTHAKIHLKQKEHACPAEGCDRRFSTSHSLTEHLRIHTGHKPFQCRICMRSFSQSSSLVRHIRTHTGEKPFACEFCGRKFAREDNLHTHAKIHLKQKEKKAEKGGAPSASSAPPVSLAPVVTTCA

SEQ ID NO: 56 - protein, 631 aa, synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 57 - protein, 719 aa, synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRRDELNVHIRIHTGQKPFQCRICMRNFSRSDHLTNHIRTHTGEKPFACDICGRKFARSDDLVRHTKIHLRQKDRPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 58 - protein, 627 aa, synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLVRHIRIHTGQKPFQCRICMRNFSHRTTLTNHIRTHTGEKPFACDICGRKFAREDNLHTHIRTHTGEKPFACDICGRKFSTSHSLTEHIRIHTGQKPFQCRICMRNFSQSSSLVRHIRTHTGEKPFACDICGRKFAREDNLHTHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 59 - protein, 631 aa, synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 60 - protein, 473 aa, synthetic construct
MTGKLAEKLPVTMSSLLNQLPDNLYPEEIPSALNLFSGSSDSVVHYNQMATENVMDIGLTNEKPNPELSYSGSFQPAPGNKTVTYLGKFAFDSPSNWCQDNIISLMSAGILGVPPASGALSTQTSTASMVQPPQGDVEAMYPALPPYSNCGDLYSEPVSFHDPQGNPGLAYSPQDYQSAKPALDSNLFPMIPDYNLYHHPNDMGSIPEHKPFQGMDPIRVNPPPITPLETIKAFKDKQIHPGFGSLPQPPLTLKPIRPRKYPNRPSKTPLHERPHACPAEGCDRRFSRSDNLVRHLRIHTGHKPFQCRICMRSFSHRTTLTNHIRTHTGEKPFACEFCGRKFAREDNLHTHAKIHLKQKEHACPAEGCDRRFSTSHSLTEHLRIHTGHKPFQCRICMRSFSQSSSLVRHIRTHTGEKPFACEFCGRKFAREDNLHTHAKIHLKQKEKKAEKGGAPSASSAPPVSLAPVVTTCA

SEQ ID NO: 61 - protein, 719 aa, synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSDPGALVRHIRIHTGQKPFQCRICMRNFSRSDNLVRHIRTHTGEKPFACDICGRKFAQSGDLRRHTKIHLRQKDRPYACPVESCDRRFSTHLDLIRHIRIHTGQKPFQCRICMRNFSTSGNLVRHIRTHTGEKPFACDICGRKFARSDNLVRHTKIHLRQKDRPYACPVESCDRRFSQSGHLTEHIRIHTGQKPFQCRICMRNFSERSHLREHIRTHTGEKPFACDICGRKFAQAGHLASHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 62 - protein, 627 aa, synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLTRHIRIHTGQKPFQCRICMRNFSHSTTLTNHIRTHTGEKPFACDICGRKFARSDNRKTHIRTHTGEKPFACDICGRKFSTSHSLTEHIRIHTGQKPFQCRICMRNFSQSSSLTRHIRTHTGEKPFACDICGRKFARSDNRKTHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 63 - protein, 473 aa, synthetic construct
MTGKLAEKLPVTMSSLLNQLPDNLYPEEIPSALNLFSGSSDSVVHYNQMATENVMDIGLTNEKPNPELSYSGSFQPAPGNKTVTYLGKFAFDSPSNWCQDNIISLMSAGILGVPPASGALSTQTSTASMVQPPQGDVEAMYPALPPYSNCGDLYSEPVSFHDPQGNPGLAYSPQDYQSAKPALDSNLFPMIPDYNLYHHPNDMGSIPEHKPFQGMDPIRVNPPPITPLETIKAFKDKQIHPGFGSLPQPPLTLKPIRPRKYPNRPSKTPLHERPHACPAEGCDRRFSRSDNLVRHLRIHTGHKPFQCRICMRSFSREDNLHTHIRTHTGEKPFACEFCGRKFARSDELVRHAKIHLKQKEHACPAEGCDRRFSQSGNLTEHLRIHTGHKPFQCRICMRSFSTSGHLVRHIRTHTGEKPFACEFCGRKFAQNSTLTEHAKIHLKQKEKKAEKGGAPSASSAPPVSLAPVVTTCA

SEQ ID NO: 64 - protein, 627 aa, synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLTRHIRIHTGQKPFQCRICMRNFSRSDNLTTHIRTHTGEKPFACDICGRKFARSDERKRHIRTHTGEKPFACDICGRKFSQSGNLTEHIRIHTGQKPFQCRICMRNFSTSGHLTRHIRTHTGEKPFACDICGRKFAQSSTRKEHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 65 - protein, 627 aa, synthetic construct
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHIRTHTGEKPFACDICGRKFSQSGNLTEHIRIHTGQKPFQCRICMRNFSTSGHLVRHIRTHTGEKPFACDICGRKFAQNSTLTEHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 66 - protein, 272 aa, synthetic construct
MAPKKKRKVGIHGVPAALEPGEKPYKCPECGKSFSSPADLTRHQRTHTGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGKKTSKRPAATKKAGQAKKKKGSYPYDVPDYALEDALDDFDLDMLGSDALDDFDLDMLGSDALDDFDLDMLGSDALDDFDLDML

SEQ ID NO: 67 - protein, 272 aa, synthetic construct
MAPKKKRKVGIHGVPAALEPGEKPYKCPECGKSFSDPGALVRHQRTHTGEKPYKCPECGKSFSRSDNLVRHQRTHTGEKPYKCPECGKSFSQSGDLRRHQRTHTGEKPYKCPECGKSFSTHLDLIRHQRTHTGEKPYKCPECGKSFSTSGNLVRHQRTHTGEKPYKCPECGKSFSRSDNLVRHQRTHTGKKTSKRPAATKKAGQAKKKKGSYPYDVPDYALEDALDDFDLDMLGSDALDDFDLDMLGSDALDDFDLDMLGSDALDDFDLDML

SEQ ID NO: 68 - protein, 261 aa, synthetic construct
MAPKKKRKVGIHGVPAALEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTSKRPAATKKAGQAKKKKGSDALDDFDLDMLGSDALDDFDLDMLGSDALDDFDLDMLGSDALDDFDLDML

SEQ ID NO: 69 - protein, 386 aa, synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 70 - protein, 402 aa, synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGGGSGGGSGQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 71 - protein, 569 aa, synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 72 - protein, 585 aa, synthetic construct
MAADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGADHLMLAEGYRLVQRPPSAAAAHGPHALRTLPPYAGPGLDSGLRPRGAPLGPPPPRQPGALAYGAFGPPSSFQPFPAVPPPAAGIAHLQPVATPYPGRAAAPPNAPGGPPGPQPAPSAAAPPPPAHALGGMDAELIDEEALTSLELELGLHRVRELPELFLGQSEFDCFSDLGSAPPAGSVSCGGSGGGSGQSQLIKPSRMRKYPNRPSKTPPHERPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDK

SEQ ID NO: 73 - protein, 402 aa, synthetic construct; identical to SEQ ID NO: 70

SEQ ID NO: 74 - protein, 569 aa, synthetic construct; identical to SEQ ID NO: 71

SEQ ID NO: 75 - protein, 585 aa, synthetic construct; identical to SEQ ID NO: 72

SEQ ID NO: 76 - protein, 16 aa, synthetic construct
MELELDAGDQDLLAFL

SEQ ID NO: 77 - protein, 530 aa, synthetic construct
MELELDAGDQDLLAFLLEESGDLGTAPDEAVRAPLDWALPLSEVPSDWEVDDLLCSLLSPPASLNILSSSNPCLVHHDHTYSLPRETVSMDLESESCRKEGTQMTPQHMEELAEQEIARLVLTDEEKSLLEKEGLILPETLPLTKTEEQILKRVRRPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDVYVGGLESRVLKYTAQNMELQNKVQLLEEQNLSLLDQLRKLQAMVIEISNKTSSSSTCILVLLVSFCLLLVPAMYSSDTRGSLPAEHGVLSRQLRALPSEDPYQLELPALQSEVPKDSTHQWLDGSDCVLQAPGNTSCLLHYMPQAPSAEPPLEWPFPDLFSEPLCRGPILPLQANLTRKGGWLPTGSPSVILQDRYSG

SEQ ID NO: 78 - protein, 530 aa, synthetic construct
MELELDAGDQDLLAFLLEESGDLGTAPDEAVRAPLDWALPLSEVPSDWEVDDLLCSLLSPPASLNILSSSNPCLVHHDHTYSLPRETVSMDLESESCRKEGTQMTPQHMEELAEQEIARLVLTDEEKSLLEKEGLILPETLPLTKTEEQILKRVRRPYACPVESCDRRFSRSDNLVRHIRIHTGQKPFQCRICMRNFSHRTTLTNHIRTHTGEKPFACDICGRKFAREDNLHTHTKIHLRQKDRPYACPVESCDRRFSTSHSLTEHIRIHTGQKPFQCRICMRNFSQSSSLVRHIRTHTGEKPFACDICGRKFAREDNLHTHTKIHLRQKDVYVGGLESRVLKYTAQNMELQNKVQLLEEQNLSLLDQLRKLQAMVIEISNKTSSSSTCILVLLVSFCLLLVPAMYSSDTRGSLPAEHGVLSRQLRALPSEDPYQLELPALQSEVPKDSTHQWLDGSDCVLQAPGNTSCLLHYMPQAPSAEPPLEWPFPDLFSEPLCRGPILPLQANLTRKGGWLPTGSPSVILQDRYSG

SEQ ID NO: 79 - protein, 380 aa, synthetic construct
MELELDAGDQDLLAFLLEESGDLGTAPDEAVRAPLDWALPLSEVPSDWEVDDLLCSLLSPPASLNILSSSNPCLVHHDHTYSLPRETVSMDLESESCRKEGTQMTPQHMEELAEQEIARLVLTDEEKSLLEKEGLILPETLPLTKTEEQILKRVRLEPGEKPYKCPECGKSFSRSDNLVRHQRTHTGEKPYKCPECGKSFSREDNLHTHQRTHTGEKPYKCPECGKSFSRSDELVRHQRTHTGEKPYKCPECGKSFSQSGNLTEHQRTHTGEKPYKCPECGKSFSTSGHLVRHQRTHTGEKPYKCPECGKSFSQNSTLTEHQRTHTGKKTSVYVGGLESRVLKYTAQNMELQNKVQLLEEQNLSLLDQLRKLQAMVIEIS

SEQ ID NO: 80 - protein, 380 aa, synthetic construct
MELELDAGDQDLLAFLLEESGDLGTAPDEAVRAPLDWALPLSEVPSDWEVDDLLCSLLSPPASLNILSSSNPCLVHHDHTYSLPRETVSMDLESESCRKEGTQMTPQHMEELAEQEIARLVLTDEEKSLLEKEGLILPETLPLTKTEEQILKRVRRPYACPVESCDRRFSRSDNLVRHIRIHTGQKPFQCRICMRNFSREDNLHTHIRTHTGEKPFACDICGRKFARSDELVRHTKIHLRQKDRPYACPVESCDRRFSQSGNLTEHIRIHTGQKPFQCRICMRNFSTSGHLVRHIRTHTGEKPFACDICGRKFAQNSTLTEHTKIHLRQKDVYVGGLESRVLKYTAQNMELQNKVQLLEEQNLSLLDQLRKLQAMVIEIS

SEQ ID NO: 81 - DNA, 681 bp, Homo sapiens
  source 1..681: mol_type = genomic DNA
ggaggaagccatcaactaaactacaatgactgtaagatacaaaattgggaatggtaacatattttgaagttctgttgacataaagaatcatgatattaatgcccatggaaatgaaagggcgatcaacactatggtttgaaaagggggaaattgtagagcacagatgtgttcgtgtggcagtgtgctgtctctagcaatactcagagaagagagagaacaatgaaattctgattggccccagtgtgagcccagatgaggttcagctgccaactttctctttcacatcttatgaaagtcatttaagcacaactaactttttttttttttttttttttttgagacagagtcttgctctgttgcccaggacagagtgcagtagtgactcaatctcggctcactgcagcctccacctcctaggctcaaacggtcctcctgcatcagcctcccaagtagctggaattacaggagtggcccaccatgcccagctaatttttgtatttttaatagatacgggggtttcaccatatcacccaggctggtctcgaactcctggcctcaagtgatccacctgcctcggcctcccaaagtgctgggattataggcgtcagccactatgcccaacccgaccaaccttttttaaaataaatatttaaaaaattggtatttcacatatatactagt

SEQ ID NO: 82 - DNA, 502 bp, Mus musculus
  source 1..502: mol_type = genomic DNA
agtttggacaagaactatagttctagctttctctgggtctccaccttgcagagaatgcagctttcattatctcatgagccaaactctcatcatctctttccatatatctgtcggtgctcttccatgagtactctaacacacacagaaggagcacttacacaggctgttgtttttctcttattatcatagctgttgttcagacatgtgcattctgttcttgttgcttcaatgctaaaggagtctcaggatatgagaactgtaccagccgaggcatcaggaaacatgggtggaaattcccacagtactatttgttcactgtgtgaccttgggccagtcacatccctttcctgaggcttcgattccccaagctataaaagaagcatctcttaacctttttttaggtcatgagtcaggcccagcacactctcagggagactcatgagagtacagatcatttcccatagaaaaaccatagttttatatccagaggcttttctgtaag

SEQ ID NO: 83 - DNA, 823 bp, Mus musculus
  source 1..823: mol_type = genomic DNA
ggttccagttcagaggcagagcatttggggttcccagtcaggagctttcctctctccgctccttagtttcctctctttaaaaaaaaatgggtgatagtatagaaaggaagctctgggctcggggaccagggccctgggatccccgctcccagccactcgctcctgacccttccagggacaagctcccccccaccccgtcctttccaggctgccactagaagagatggggacgcgtggtcagccgcttctgtcgccccccagggaacggtctcacgctggagggggcagtgccctcggaacaggacagtcagcccaagccagccaagcgcgcgcggacgtccttcaccgcagagcaattgcaggtaccccgggcaagccccgaagcgtgtgggcggggcttcggagtgggcgtggttgttcgggacttgtgactccgccccttgtgcggggacccgcgtgaggccgctccaaggatgaagctgcctggggcgtggcctcggaccctgagcctctgattgggcggaggtctcagggcccttctgcgccccacaggttatgcaggcgcagttcgcgcaggacaacaacccggacgcgcagacgctgcagaagctggcggacatgacgggcctcagtcgcagggtcatccaggtggggctccggggtctcggccttcaggtctagggtgaaccttagggaagcgctgaagctcgtagtggtacggatggtcgcgcgtgcacgtggccgcccctctccagtgtggcctaaggaccccagtcggcacgggttgacccttttccttgattactgagagtgcagaggctgt

SEQ ID NO: 84 - DNA, 633 bp, Mus musculus
  source 1..633: mol_type = genomic DNA
tggtgggaagacatgtccagggaagaaatggcctccagaggcctgaggtggggaaatgctggaggtggagagaggaacaactgactgaaaatgagcttccactgtggcttagtagcctataccaagtctagagtatagggtaggagaagattaggaaagcgatgggtctgagaatgatgtggcctgttgacttttgtaaacccaaagcaccttggactaaaccctatgaacagtgtggtgccaccaaagactataatgagctcagggaacagaattctgtgtgcatggtgattttttttttttttttctgctaactgcagtctgggtgatgcattgacaaaccaatcctggaaagtaagaggcaagggcagctgggacggtgagaggagcctgatgggaaccaggccaagcagggcagcagaggcgatgaagaggatgtggtgcatccagagactcacttcattagctggaggcactgctggatagggtctgaaggttctggtatctgagttggcgggctgggtgagtggtggctctgcttcctgaacagtgtgtgcaagaggaaacagggttaagggctaggacagtcacaggtgagtcagcctcacaagagcaaccttcccctagtgcaga

SEQ ID NO: 85 - DNA, 670 bp, Mus musculus
  source 1..670: mol_type = genomic DNA
ggaggtctccttttgccccggttccaacaagagaatgcaaggctgtatctcaatttccttgagcctctctgtattatagaagaaaagtagggaagccatacgccccttctgagcttcagtgtctctctgtctctgcaaatgaggctggggaggctgggggcgggcgtgaaagaggcccgcgccaagccgacccccacctctgccccctccccaggtcaacaacctcatctggcacgtgcggtgcctcgagtgctccgtgtgtcgcacatcgctgaggcagcagaatagctgctacatcaagaacaaggagatctactgcaagatggactacttcaggtaggcagcggccatcccgccagcaagcgctggagcatgaacgccttgcacacgcgtgcctaggccacttgtgtggcctgtgctctccaattcctgagccctgctgttcagagtgcacaacgcggctcagcgcactggcccggccctcctactcagcacgtcttacacagaagggagcgccagtctcagcctgagttctggcgggggatctgcctcgggttcctccgatctgacaggcgctggccacgggtctggttccatctctggtcttttctggccccgagcaccagtgtgttctgttgagctctgatgtccgaggctctggcccggatca

SEQ ID NO: 86 - DNA, 1043 bp, Mus musculus
  source 1..1043: mol_type = genomic DNA
ctctggctacctcttatcttgggcattcacgacaatttctaattgcaggtagtttgtgtgtgtgcgcgtgttttttttccccctcagaggcttggattgcaaaggaactaagcgattacttcaagagccacgggttaagtgcagggagagggggagagagagggaaaaaaacccaatccaaattcaaattgcttcattagagagacaccgcttttgtggggaagggctttaaatgcccactacaaagttaggactcattgttcagcgccggtttatataacaggcgaggggaggcgctgggctctgacagctccgagccagttcagcagccgccgtcgcctgcattccctccccctcccccaggtgatggcccagccagggtccggctgcaaagcgaccacccgctgtctcgaagggaccgctccgcctgccatggtgagtcctttcggtcctgctttcggccccgagtccccccaacagcacaggccagggcttctggctcagccttccggctaccaacctctacccctgcgctggaaaactgccgataggagccgcctctcgttgagccttggtttttctggcctggaatgtgagctttggctgcttcctgcacccaggatgcgctgtgttaaaagttgggggccgtcccttcttctccaataggtcctttcattcttgtactccagcctagggcgcgacatccctggcacatttcggtgtcagtcggtgcgcgaggaaaccagattcaactctgagtactcggctaagcgcttcgctgttcctctctcccatttcaggctcagtcagacgcagaggccttggcaggcgctctggacaaggacgaaggtagagcctccccatgtacgcccagcacaccgtctgtctgctcgccgccctctgctgcctcttccgtgccgtctgccggcaagaatatctgctccagttgcggtctggagatcctggaccggtatctgctcaaggtgagtcagggtaggtgtgcctgcttgcccacgggtgtggtttgcagccccaagagctgt

SEQ ID NO: 87 - DNA, 507 bp, Mus musculus
  source 1..507: mol_type = genomic DNA
caagacttttaaaagtttagataaataaacaaacatttgacggctttccatcacatctagactataatccaaagatctatatggtcccaaacgacttacacttaactaccgtctcccatatggcttcttcccccatcagtcattgtcctcagccatagtggcctccctgttcctttgggtacaagggaacaactccctgagaggttccattagctgctgttgcctgagatgctcttgagcccacaccatctgctcatttctctcctcacgtgtcagtgattaagaggctgtccttggcctcccgtcaaaattacatccctgccgctttccacttcttgccttcttattttctaaatagaactaactcaccactacccaacattctatataattggatatctgtcctctgtttaaatataatgttgacttcaagaaagaacgttgtcactgccctgtcaccagacttttaaacagtgcctatcgtgtggcacatgctcagtgaaattg

SEQ ID NO: 88 - DNA, 502 bp, Mus musculus
  source 1..502: mol_type = genomic DNA
tcaacagggggacacttgggaaagaaggatggggacagagccgagaggactgttacacattagagaaacatcagtgactgtgccagctttggggtagactgcacaaaagccctgaggcagcacaggcaggatccagtctgctggtcccaggaagctaaccgtctcagacagagcacaaagcaccgagacatgtgccacaaggcttgtgtagagaggtcagaggacagcgtacaggtcccagagatcaaactcaacctcaccaggcttggcagcaagcctttaccaacccacccccaccccacccaccctgcacgcgcccctctcccctccccatggtctcccatggctatctcacttggccctaaaatgtttaaggatgacactggctgctgagtggaaatgagacagcagaagtcaacagtagattttaggaaagccagagaaaaaggcttgtgctgtttttagaaagccaagggacaagctaagatagggcccaagtaat

SEQ ID NO: 89 - DNA, 788 bp, Mus musculus
  source 1..788: mol_type = genomic DNA
aaatagaactgtgagatagggggagagggggcaggaaggacaagagacccctgtctcattgtgatccccacctgtctgctctgtgggagggtacccatgagggccagcccacagcccttaggtggacattgtctggtcctgtctcactgtccctcccagcagccccagaggccaggagacaggggtctcagtcctcactgagagatgtgtaaactgaggcccagtgaatgttgagggccagggcatgcccttggtgggatgtgacctgggtctccttcgcacgggcttcctccccgaagccgagctgagcatttggagtttgaaatgtttccgtacttagcaatctgctcctctattcccgggcggacttccgatagctccggccttatgctgcactagataagatggagcagggagaggacacggcactacttatgtaaccggcctcttgaaaaatggagcagcggtcagggcggaacaagacgtcctctctctacgcatccctctcctttccctgctaaggctgcagctggagtcagaggcagggctgttccaatctgtctttgatcagtaacgcagccagcctccagcctccgtcagcctcctcatggctgagacccggcctcagtttcccccacttacatcccgaggatcagagcctgtgaggatgaaatgggataaggtagctggaaccgtctggcagagagcgagtcctcaggactgttgatgcctgtggctgcctggcttgaccccaagtgaccccgcctcctcatcctgcagcaggagaa

SEQ ID NO: 90 - DNA, 502 bp, Mus musculus
  source 1..502: mol_type = genomic DNA
tctatagaatgtgtccccagccttgttttccacacttgatacgcaaggaatgcataccacagagagggatgagggtagcatccagcctgcttcctgtgtgtcggggcgctacagccacatctccccagtccatctcagaccgtcacagagcttcgccgaatgtatagctttgttctctgtgcagacagggagacagagccttgggaagcataggtgcttgcttctttgcccactgagtcttagctggacttgcacaccacatgcctcacagccgggcgcacttgcatttgtcacccaggcccagtgatgatggctctgcttgctttgtgctttgtgccaactacagctccagcacctgtgccctgggttttcactcctttagttgaacacgtagttactggggttgtagggatggagcctttctgcttccttctggcaaagtccttagcggcctgctgcgggggtggggggtgttcaggggagtggtgatgaagtatgacag

SEQ ID NO: 91 - DNA, 533 bp, Mus musculus
  source 1..533: mol_type = genomic DNA
tctccagttggagaaacagatgctgtaactggggccacagtataaagagagcccagacattgaactgtcaacacagaagcctggcacactggaactggcagtccagctgggaacaaggggtagaggctgaggccactaagtcaactgaggcaggagacataggagctaaagcagctgaagggtgcaggacagctggggggtctgaagtgggcctcatgcccagagctatgaagtcaggggctgtagcctaggagccttggaagccagctggcaagctgtggcccaaagacgctgactcaccaggagggggcagctggagccaggcactcctaaggtttccaggaagggcagccttccagggctcagctaggggagacagtgttgacagcaagttgtcaggcaacttgagctactgggcagctgggaagctgtcccttggtccccagtatcatcatcaccccagacgctgcccacctgcctcaggtcccacacagtgatcctcccatctttaacacaacacatgaccagagaga

SEQ ID NO: 92 - DNA, 524 bp, Mus musculus
  source 1..524: mol_type = genomic DNA
gtcaccctccccccaaacaaccccttcttctctggttcgagaaattacaggcatgaaagatataaatcgggatgcttgacttgggaatataaatcactaaagcttgggggcaggggtgggcgacctttgtgaccgtccttgtgcgtgccagtaaatcctgtggtccaggggagaagaaaaggctgtgtggcttctgctcacaaagctgcagaaaccattctttaagcccaaaagcacttccagagagagcagagcatccccaggctgctggctcagcaagttcactgtgctcaatctcaggaagtgaggataagagcagtgcctggagagtgcctggtgctgagctgagggtttctgaacacattaaagcggggagcatggaccgggcctcaggaggggtgttgaacatccctaggcagaggagtctagcttcctgggaaaagatatcaggttaagcacacacatgtcctctggaataagataatctttctgatcacacactatacacacacaaaagcctgctc

SEQ ID NO: 93 - DNA, 509 bp, Mus musculus
  source 1..509: mol_type = genomic DNA
gccctctaggccacctgaccaggtcccctcagtcccccccttcccacactcccacactcagcccccctcccccccccccgacccctgcaggattatcctgtctgtgttcctgactcagcctgggagccacctgggcagcaggggccaagggtgtcctagaagggacctggagtccacgctgggccaagcctgccctttctccctctgtcttccgtccctgcttgcggttctgctgaatgtggttatttctctggctccttttacagagaatgctgctgctaattttatgtggagctctgaggcagtgtaattggaagccagacaccctgtcagcagtgggctcccgtcctgagctgccatgcttcctgctctcctcccgtcccggctcctcatttcatgcagccacctgtcccagggagagaggagtcacccaggcccctcagtccgccccttaaataagaaagcctccgttgctcggcacacataccaagcagccgctggtgcaatct

SEQ ID NO: 94 - DNA, 398 bp, Mus musculus
  source 1..398: mol_type = genomic DNA
gtgttcttcccttcccctttggacccccgagacaagccaataaaatactcggcagggtggcttctctcctttttttgccagtaataaacagactcagagcaagttaagggtctggtccaaggtcatggctgggatcagtgacagagcccagaagagaacctgagacttcttgctgagccaagctggagaggacagaaaggaatgcgtctactccatgcatgaccctctgccagctttgctccttcctaagggaccatgaacgatatgtgcacaccgctcatacgtatgtgcacacctgcaagaggaggcatcccatgtacacctatgagacgcacagagaaacatatatgtagccataggctagaaattctttctctttctaggtctgcccctctgca

SEQ ID NO: 95 - DNA, 810 bp, Mus musculus
  source 1..810: mol_type = genomic DNA
ggaccactcagtgtacacggaatgtagaattgagtctgccattggtcttccctcaaagtcttggaggcttgggactgatattgggagcatctgggcagagaaggccacaaagacagggtggtttttctacactgggacatactcgtgagcatgcacagaggcgtgtccccaacttccctgtcacccctgtcctctgccggctagaggggatgcgggggtggacatatgctgctattgggcagatatcacatgttaagaggtgggggggggctcaagaggcggagggctaggagcatcccatggggagaggttctggttttcttgctgcctctagctgctataaatacgttagcacttgagcaactggaaagctctgagtaatttaggatgcacaaagctgtaatttaactccagcatctcagtgtgcgagagcattaaagatgtaattaagatgtttacacaaagagattggagtctgtgacacttggggtgcaaaaccccaggaagggacacaatgggtgaggtgaggatctgtgggaggcctggggacagtcacttggatcccagctatgagatggcaggccacccagctgtttctccttggaaatgttttggcctgggggttgggggtggggcatcacactttgatatggagatggggcaacaaagcctgcaatatctgggggtggagaggtcaagtggatggagtcttttgagatcatgtcaggaagagggctcgatcccccaaaatcatggtgacatatggtgtctcggggttcacaggagctatgtctaaaatacaaaagtaaa

SEQ ID NO: 96 - DNA, 202 bp, Mus musculus
  source 1..202: mol_type = genomic DNA
tctgcagaagcctgccattccaccatttaaacctgtgactccaggccttaagcctgttgaaggtcgagtcccagaagggtcatatgtgcaactgcctagggagagttcccactcgcagggccaagaggagtcccccggtctgaggtgtgggggcggggacgtgcactgggcgctgggaccacggctggggctcaggactcgc

SEQ ID NO: 97 - DNA, 937 bp, Mus musculus
  source 1..937: mol_type = genomic DNA
tgcctcagtttcttcgcctagaaagccgggtctaagggtacatgccctgattcttttctggggtgtctcgaattttaaacaacacatactgttctgggctgatgacaagaggaagtactggtcggtggctgatggacatccaccatggtggcaactggagggagggggaacggacgttgaaaccctgccctcctggaatctgtcgcatgcacgcacgttgacaatgcttggcactggggacaggctgggatggatggagcggagcgtgaggaggagtgggcatgcaggcccgagtgtctgttttgctgattgctccttttgctttcaaggagattaaactatttttagtccatgcctactgctggtgagacgctggaggaagcctttccatcgttgagattttctggaagctgccaagtgtggtcttcagctcaattctgggagcctcccagagtgggagggaggaacatttccatctgggggcttcggggacaggctaagatcttccctggggtccttgctgcgctggcctcctcaaaccacgctgcctcggcctgcataaagcagtaatctgatgtgcccgatgtttgtaacgctgtgtttaaaaaaagtaatttattttctaattattccttgtcttgcataaccatgcattgccaaagtgtcgctatttaaaatatttatctctccacgccgcaggagcagctctggagcgtggagggggaagaaataaaagtccgcgtgccagtcgcaggcatattactttgactcgtcctggtggctttgacgtctccctgtaaatacatttatttttcattaggacgtttctgagcttgtggcccccggagagcggagtgattacgctgttcatctgcaagcgatgcaatagaggggtactcgcagaatgacttccgcccagagcatcctgcgcctgtct

SEQ ID NO: 98 - DNA, 922 bp, Mus musculus
  source 1..922: mol_type = genomic DNA
taaaataccttatttttttccagtctctaaactgctaatctcccaggctaagggattctgggacaaaggcaaggcctggaagtggaaatctgtaaaattagcttcagcggtattagtgtttgcagttgaagattgaaaaactgctttcccagggcctgattggaggctccactctcctccaggaagaggcaaggactctgggctggcactgaggacaaatcctgggaggctgctatggggcctgggagccaggctgccttgtgctagaggcctagagagtgtctgtgtcccaagtcccaagctacccccagcagctaacagcttttccagttctcaggcacagcaggtgccaagatcacgctctggagtccagctgggccccttcctcttctttttttttttttttttttaagacctcctggacactgttcctctccccccccccgtgaccccccccctcagttctcaaacacgtgagggttgggggagggttccacagccagagagaggggccagctctggtgcctgtgggtacgcccgcccgtatggcccatcaggcctcttgtgtgcttgattgcctctgattggctgcagctgaattcagcaaaagctattatttgcccttgatgagccaatcagatggcctcattggccattcagagcaggcaccggaacctgagggtggggtggggggtgggggatggagatgggactcagtgagggggtgggaagctctaaaacagatgcaggacctgagcctgtctgtgtccaccacgaccttcacacaggtcacacccccttcccctgacttgtcaccccaaaccagggcttgttgcccaaccccacctcacaattccctcactctgtaacacctttccatatacctctgcatgtctaaacccaagacttgctctatgaaatc

SEQ ID NO: 99 - DNA, 639 bp, Mus musculus
  source 1..639: mol_type = genomic DNA
agaccctgcttagcacagctcttagcgggtcctttagggggtctcccagcgggcccagtgggaatgagataaggaaggacacagctgtccattctcccgtgcctgctaaggaggaaatggggccgccttacataattggggcaatttgttccactcttgtcctcctggtatcatggctatcaccccctccttgctcagggagtccttgattgagcgagaagctcaggcctccctctctccctcctgctgggggttgctgaacagagggtgtaggagccataggctctgtcactgctgagatctgccagatgtctaggccaggagaaaatggaaagggctaagtcacagcatatgtggccactcaggcctatagccccaaatctgcctggtaacccattatgtccccagagaatttgcatgggcggacaccctcatgccgggtctcagtaagggaaggggtgggaggcaaaaatatccctccccaccctgaatctccaccccctccccccagaaactgacacttggccttgtctaaggatgggttttcccaaaatccttctgaaaaaaacagaatttcaagagtcactccctccgggtctcagcctagaacatatgcagtatcccctgacgtccataggg

SEQ ID NO: 100 - DNA, 716 bp, Mus musculus
  source 1..716: mol_type = genomic DNA
aaactggcacagtaatggcgggctgacagacaagggagtctgtagcacccgctgcctccgcccaccccttctccgagcaattaaaaggtgtttatgtggggctggcagtggcttctgcctcccttccattacgaacattaagagatcttgacccttccactttccccgctcttgaaaggagctgcagacacgtggagccaattaggcgcacgcgtgggcgccaagggcctgagcagctttttctccctgattgcggcgtttacagctgattattctcccctcacccaaacagtgctgcttcctggcaaggtgccacccagaggagccggctgggggcccctggggacaggggaggactggattagtaaatgggcatctatcgaatggctttcatatgtgtggctggaagggagaagggtagggccaggaatggtggcagcaagggcccaggtagcaatgagggttcttctaacccaccatttagggatagcgatcagaaaagggccctcgaggaggtgacctaaatgtgtgtagaagctgacggccactacacacacacacacacacacacacacacatacacaagcatccttgtccttggagtcggtcagcatgagcaagagaaagatgttcccagtggccatgagagtggagccctcctccctacttacatccaggttggatggccaggagatcctgagatccttcaagactcc

SEQ ID NO: 101 - DNA, 147 bp, Mus musculus
  source 1..147: mol_type = genomic DNA
aagccacatcctgggtggaaatatatggcttcaattcccactcttccggatgacctctgtggggagccctggcttcaccttggtccagcttcatcccttagcctcgctgccaggaaggcagtgaggtcagaggctggtgctggcgtg

SEQ ID NO: 102 - DNA, 297 bp, Mus musculus
  source 1..297: mol_type = genomic DNA
cctacctggtgcccgccaacatctgggggccatcctggccagcgccagcgtggtggtgaaggcactgtgcgccgtggtactgtttctctacctgctttccttcgctgtggacacgggctgcctggccgtcaccccaggctaccttttcccacccaacttctggatctggaccctggccacccacgggctcatggaacagcacgtgtgggacgtggccattagcctggccacagtggttgtggccgggcgattactggagcccctctggggagccttggagctgctcatcttcttctc

SEQ ID NO: 103 - DNA, 101 bp, Homo sapiens
  source 1..101: mol_type = genomic DNA
aaacggacgggcctccgctgaaccagtgaggccccagacgtgcgcataaataacccctgcgtgctgcaccacctggggagagggggaggaccacggtaaat

SEQ ID NO: 104 - DNA, 101 bp, Homo sapiens
  source 1..101: mol_type = genomic DNA
ggagcgagcgcatagcaaaagggacgcggggtccttttctctgccggtggcactgggtagctgtggccaggtgtggtactttgatggggcccagggctgga

SEQ ID NO: 105 - DNA, 101 bp, Homo sapiens
  source 1..101: mol_type = genomic DNA
gctcaaggaagcgtcgcagggtcacagatctgggggaaccccggggaaaagcactgaggcaaaaccgccgctcgtctcctacaatatatgggagggggagg

SEQ ID NO: 106 - DNA, 547 bp, Homo sapiens
  source 1..547: mol_type = genomic DNA
ttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccc

SEQ ID NO: 107 - DNA, 101 bp, Homo sapiens
  source 1..101: mol_type = genomic DNA
cagcagccgaaggcgctactaggaacggtaacctgttacttttccaggggccgtagtcgacccgctgcccgagttgctgtgcgactgcgcgcgcggggcta

SEQ ID NO: 108 - DNA, 201 bp, Homo sapiens
  source 1..201: mol_type = genomic DNA
gagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctcccc

SEQ ID NO: 109 - DNA, 199 bp, Homo sapiens
  source 1..199: mol_type = genomic DNA
gctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctg

SEQ ID NO: 110 - DNA, 1351 bp, Homo sapiens
  source 1..1351: mol_type = genomic DNA
aaacggacgggcctccgctgaaccagtgaggccccagacgtgcgcataaataacccctgcgtgctgcaccacctggggagagggggaggaccacggtaaatggagcgagcgcatagcaaaagggacgcggggtccttttctctgccggtggcactgggtagctgtggccaggtgtggtactttgatggggcccagggctggagctcaaggaagcgtcgcagggtcacagatctgggggaaccccggggaaaagcactgaggcaaaaccgccgctcgtctcctacaatatatgggagggggaggttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccccagcagccgaaggcgctactaggaacggtaacctgttacttttccaggggccgtagtcgacccgctgcccgagttgctgtgcgactgcgcgcgcggggctagagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccgctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctg

SEQ ID NO: 111 - DNA, 2051 bp, synthetic construct
  source 1..2051: mol_type = other DNA
ggaggaagccatcaactaaactacaatgactgtaagatacaaaattgggaatggtaacatattttgaagttctgttgacataaagaatcatgatattaatgcccatggaaatgaaagggcgatcaacactatggtttgaaaagggggaaattgtagagcacagatgtgttcgtgtggcagtgtgctgtctctagcaatactcagagaagagagagaacaatgaaattctgattggccccagtgtgagcccagatgaggttcagctgccaactttctctttcacatcttatgaaagtcatttaagcacaactaactttttttttttttttttttttttgagacagagtcttgctctgttgcccaggacagagtgcagtagtgactcaatctcggctcactgcagcctccacctcctaggctcaaacggtcctcctgcatcagcctcccaagtagctggaattacaggagtggcccaccatgcccagctaatttttgtatttttaatagatacgggggtttcaccatatcacccaggctggtctcgaactcctggcctcaagtgatccacctgcctcggcctcccaaagtgctgggattataggcgtcagccactatgcccaacccgaccaaccttttttaaaataaatatttaaaaaattggtatttcacatatatactagtatttacatttatccacacaaaacggacgggcctccgctgaaccagtgaggccccagacgtgcgcataaataacccctgcgtgctgcaccacctggggagagggggaggaccacggtaaatggagcgagcgcatagcaaaagggacgcggggtccttttctctgccggtggcactgggtagctgtggccaggtgtggtactttgatggggcccagggctggagctcaaggaagcgtcgcagggtcacagatctgggggaaccccggggaaaagcactgaggcaaaaccgccgctcgtctcctacaatatatgggagggggaggttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccccagcagccgaaggcgctactaggaacggtaacctgttacttttccaggggccgtagtcgacccgctgcccgagttgctgtgcgactgcgcgcgcggggctagagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccgctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctg

SEQ ID NO: 112 - DNA, 1878 bp, synthetic construct
  source 1..1878: mol_type = other DNA
tcaacagggggacacttgggaaagaaggatggggacagagccgagaggactgttacacattagagaaacatcagtgactgtgccagctttggggtagactgcacaaaagccctgaggcagcacaggcaggatccagtctgctggtcccaggaagctaaccgtctcagacagagcacaaagcaccgagacatgtgccacaaggcttgtgtagagaggtcagaggacagcgtacaggtcccagagatcaaactcaacctcaccaggcttggcagcaagcctttaccaacccacccccaccccacccaccctgcacgcgcccctctcccctccccatggtctcccatggctatctcacttggccctaaaatgtttaaggatgacactggctgctgagtggaaatgagacagcagaagtcaacagtagattttaggaaagccagagaaaaaggcttgtgctgtttttagaaagccaagggacaagctaagatagggcccaagtaatgctagtatttacatttatccacacaaaacggacgggcctccgctgaaccagtgaggccccagacgtgcgcataaataacccctgcgtgctgcaccacctggggagagggggaggaccacggtaaatggagcgagcgcatagcaaaagggacgcggggtccttttctctgccggtggcactgggtagctgtggccaggtgtggtactttgatggggcccagggctggagctcaaggaagcgtcgcagggtcacagatctgggggaaccccggggaaaagcactgaggcaaaaccgccgctcgtctcctacaatatatgggagggggaggttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccccagcagccgaaggcgctactaggaacggtaacctgttacttttccaggggccgtagtcgacccgctgcccgagttgctgtgcgactgcgcgcgcggggctagagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccgctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctg

SEQ ID NO: 113 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 114 - DNA, 62 bp, synthetic construct
  source 1..62: mol_type = other DNA
aataaaagatctttattttcattagatctgtgtgttggttttttgtgtgcggaccgcacgtg

SEQ ID NO: 115 - DNA, 3872 bp, synthetic construct
  source 1..3872: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggcgcgccggaggaagccatcaactaaactacaatgactgtaagatacaaaattgggaatggtaacatattttgaagttctgttgacataaagaatcatgatattaatgcccatggaaatgaaagggcgatcaacactatggtttgaaaagggggaaattgtagagcacagatgtgttcgtgtggcagtgtgctgtctctagcaatactcagagaagagagagaacaatgaaattctgattggccccagtgtgagcccagatgaggttcagctgccaactttctctttcacatcttatgaaagtcatttaagcacaactaactttttttttttttttttttttttgagacagagtcttgctctgttgcccaggacagagtgcagtagtgactcaatctcggctcactgcagcctccacctcctaggctcaaacggtcctcctgcatcagcctcccaagtagctggaattacaggagtggcccaccatgcccagctaatttttgtatttttaatagatacgggggtttcaccatatcacccaggctggtctcgaactcctggcctcaagtgatccacctgcctcggcctcccaaagtgctgggattataggcgtcagccactatgcccaacccgaccaaccttttttaaaataaatatttaaaaaattggtatttcacatatatactagtatttacatttatccacacaaaacggacgggcctccgctgaaccagtgaggccccagacgtgcgcataaataacccctgcgtgctgcaccacctggggagagggggaggaccacggtaaatggagcgagcgcatagcaaaagggacgcggggtccttttctctgccggtggcactgggtagctgtggccaggtgtggtactttgatggggcccagggctggagctcaaggaagcgtcgcagggtcacagatctgggggaaccccggggaaaagcactgaggcaaaaccgccgctcgtctcctacaatatatgggagggggaggttgagtacgttctggattactcataagaccttttttttttccttccgggcgcaaaaccgtgagctggatttataatcgccctataaagctccagaggcggtcaggcacctgcagaggagccccgccgctccgccgactagctgcccccgcgagcaacggcctcgtgatttccccgccgatccggtccccgcctccccactctgcccccgcctaccccggagccgtgcagccgcctctccgaatctctctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgaggtgggtaagctagcgaccacctggacttcccagcgcccaaccgtggcttttcagccaggtcctctcctcccgcggcttctcaaccaaccccatcccagcgccggccacccaacctcccgaaatgagtgcttcctgccccagcagccgaaggcgctactaggaacggtaacctgttacttttccaggggccgtagtcgacccgctgcccgagttgctgtgcgactgcgcgcgcggggctagagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccgctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctggaattcgccaccatggccccaaagaagaagcggaaggtcggtatccacggagtcccagcagccctcgaaccaggtgaaaaaccttacaaatgtcctgaatgtgggaaatcattcagtcgcagcgacaacctggtgagacatcaacgcacccatacaggagaaaaaccttataaatgtccagaatgtggaaagtccttctcacgagaggataacttgcacactcatcaacgaacacatactggtgaaaaaccatacaagtgtcccgaatgtggtaaaagttttagccggagcgatgaacttgtccgacaccaacgaacccatacaggcgagaagccttacaaatgtcccgagtgtggcaagagcttctcacaatcagggaatctgactgagcatcaacgaactcataccggggaaaaaccttacaagtgtccagagtgtgggaagagcttttccacaagtggacatctggtacgccaccagaggacacatacaggggagaagccctacaaatgccccgaatgcggtaaaagtttctctcagaatagtaccctgaccgaacaccagcgaacacacactgggaaaaaaacgagtaaaaggccggcggccacgaaaaaggccggccaggcaaaaaagaaaaagggatccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttataaaaagagaccggttcactgtgacagtaaaagagaccggttcactgtgagaatgaaagagaccggttcactgtgatcggaaaagagaccggttcactgtgagcggccttgaaacccagcagacaatgtagctcagtagaaacccagcagacaatgtagctgaatggaaacccagcagacaatgtagcttcggagaaacccagcagacaatgtagctaagcttgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg